(19) United States
(12) Patent Application Publication
(10) Pub. No.: US 2015/0023926 A1
(43) Pub. Date: Jan. 22, 2015

(54) TREATMENT OF RADIATION INJURY USING AMNION DERIVED ADHERENT CELLS

(71) Applicants: Aleksandar Francki, Annandale, NJ (US); Ajai Pal, Bridgewater, NJ (US); Robert J. Hariri, Bernardsville, NJ (US); Vladimir Jankovic, New York, NY (US)

(72) Inventors: Aleksandar Francki, Annandale, NJ (US); Ajai Pal, Bridgewater, NJ (US); Robert J. Hariri, Bernardsville, NJ (US); Vladimir Jankovic, New York, NY (US)

(73) Assignee: ANTHROGENESIS CORPORATION, Warren, NJ (US)

(21) Appl. No.: 14/232,855

(22) PCT Filed: Jul. 13, 2012
(86) PCT No.: PCT/US2012/046597

§ 371 Date: Oct. 7, 2014

Related U.S. Application Data
(60) Provisional application No. 61/508,553, filed on Jul. 15, 2011.

(51) Int. Cl. A61K 35/50

(52) U.S. Cl. CPC A61K 35/50
USPC 424/93.7

(57) ABSTRACT

Provided herein are methods of treating individuals having suffered exposure to radiation, e.g., individuals having radiation injury, by administering to the individuals angiogenic cells from amnion, referred to as amnion derived adherent cells, or populations of and compositions comprising, such cells.

This application claims priority to U.S. Provisional Patent Application No. 61/508,553, the disclosure of which is herein incorporated by reference in its entirety.

### 1. FIELD

Provided herein are methods of treating individuals having radiation injury comprising administering to the individual a therapeutically effective amount of angiogenic cells from amnion, referred to herein as “amnion derived adherent cells” (AMDACs). Amnion derived adherent cells are distinct from previously-described tissue culture surface-adherent placental stem cells.

### 2. BACKGROUND

A need exists for therapies that can ameliorate or palliate the physiological effects of exposure to radiation, including injury to the body arising from exposure to radiation. Provided herein are methods of treating individuals who have been exposed to radiation comprising administration of therapeutically-effective amounts (numbers) of AMDACs.

### 3. SUMMARY

In one aspect, provided herein is a method of treating an individual who has been exposed to radiation, e.g., an individual having a radiation injury, comprising administering to the individual a therapeutically-effective amount of isolated amnion derived adherent cells (AMDACs), wherein said cells are adherent to a tissue culture surface, and wherein said cells are OCT-4⁻ (octamer binding protein 4) as determinable by RT-PCR. In certain embodiments, the AMDACs are OCT-4⁻ and CD49f⁺. In certain embodiments, said radiation is ionizing radiation. In a specific embodiment, the ionizing radiation is beta radiation, gamma radiation, or X-rays. In another embodiment, said radiation is alpha radiation. In another embodiment, said radiation is neutron radiation.

In specific embodiments, said radiation is an acute, e.g., a single, dose of between 0.01 milliSieverts (mSv) and 0.1 mSv (between 0.001 rem and 0.01 rem); an acute, e.g., a single, dose of between 1 mSv and 10 mSv (between 0.1 rem and 1.0 rem) (between 0.001 Grays (Gy) and 0.01 Gy); an acute, e.g., a single, dose of between 10 mSv and 100 mSv (between 1 rem and 10 rem) (between 0.01 Gy and 0.1 Gy); an acute, e.g., a single, dose of between 100 mSv and 1000 mSv (between 10 rem and 100 rem) (between 0.1 Gy and 1.0 Gy); an acute, e.g., a single, dose of between 1000 mSv and 2000 mSv (between 100 rem and 200 rem) (between 1 Gy and 2 Gy); an acute, e.g., a single, dose of between 2000 mSv and 3000 mSv (between 200 rem and 300 rem) (between 2 Gy and 3 Gy); an acute, e.g., a single, dose of between 3000 mSv and 4000 mSv (between 300 rem and 400 rem) (between 3 Gy and 4 Gy); an acute, e.g., a single, dose between 4000 mSv and 5000 mSv (between 400 rem and 500 rem or an acute, e.g., a single, dose of between 5000 mSv and 10000 mSv (500 rem and 1000 rem) (5 Gy and 10 Gy).

In other specific embodiments, said radiation is a chronic exposure, or substantially chronic exposure, of between 0.01 mSv and 0.1 mSv (between 0.001 rem and 0.01 rem); a chronic exposure of between 1 mSv and 10 mSv (between 0.1 rem and 1.0 rem) (between 0.001 Gy and 0.01 Gy); a chronic exposure of between 10 mSv and 100 mSv (between 1 rem and 10 rem) (between 0.01 Gy and 0.1 Gy); a chronic exposure of between 100 mSv and 1000 mSv (between 10 rem and 100 rem) (between 0.1 Gy and 1.0 Gy); a chronic exposure of between 1000 mSv and 2000 mSv (between 100 rem and 200 rem) (between 1 Gy and 2 Gy); a chronic exposure of between 2000 mSv and 3000 mSv (between 200 rem and 300 rem) (between 2 Gy and 3 Gy); a chronic exposure of between 3000 mSv and 4000 mSv (between 300 rem and 400 rem) (between 3 Gy and 4 Gy); a chronic exposure of between 4000 mSv and 5000 mSv (between 400 rem and 500 rem) (between 4 Gy and 5 Gy), a chronic exposure of between 5000 mSv and 10000 mSv (between 500 rem and 1000 rem) (between 5 Gy and 10 Gy); or a chronic exposure of between 10000 mSv and 100000 mSv (between 1000 rem and 10000 rem) (10 Gy and 100 Gy). In certain specific embodiments, said chronic exposure is over 1-6 days; over 7-13 days; over 14-27 days; over 28-56 days; or over longer than 56 days. A “substantially chronic exposure” can include, e.g., exposure over an extended period of days, weeks or months, during which exposure is not continuous but is chronic, e.g., exposure in a particular location as wind shifts from a radiation source.

In specific embodiments, the individual has been exposed to said radiation in a medical setting. In a more specific embodiment, said individual has been exposed to said radiation for the purpose of myeloablation. In more specific embodiments, said myeloablation is partial (that is, at least some of the myeloid cells of the individual are allowed survive the radiation treatment, or the dose is calculated to do so) or complete (that is, the radiation exposure is designed to kill substantially all of the myeloid cells of the individual; or the radiation exposure necessitates a stem cell transplant, e.g., a bone marrow transplant, in order to preserve the life of the individual). In other embodiments, the individual has been exposed in a non-medical setting, e.g., in the workplace.

In certain embodiments, said individual has not yet developed one or more symptoms of acute radiation syndrome at the time of said administering. In other embodiments, said individual has developed, or is likely to develop, acute radiation syndrome or a symptom of acute radiation syndrome as a result of said exposure to radiation. In specific embodiments, said one or more symptoms comprise one or more of nausea, vomiting, diarrhea, fever, and/or headache. In other specific embodiments, said one or more symptoms comprise purpuria, weakness, fatigue, infections, alopecia, blistering or necrosis of exposed tissue, and/or hemorrhage. In other specific embodiments, said one or more symptoms comprise neurological impairment, cognitive impairment, ataxia, tremors and/or seizures. In another specific embodiment, said one or more symptoms comprises leukopenia.

In another embodiment, said individual is exposed to radiation from a source not contacting the individual's body. In another embodiment, said individual is exposed to radiation as a result of a radioactive source contacting the individual's body. In a specific embodiment, said individual is exposed to radiation as a result of the individual's inhalation or ingestion of a radioactive source.

In certain specific embodiments, said administering takes place within 96 hours of said exposure; within 72 hours of said exposure; within 48 hours of said exposure; or within 24 hours of said exposure.

In another aspect, provided herein is a method of inducing hematopoietic reconstitution (e.g., partial or complete hematopoietic reconstitution) in a subject in need thereof, comprising administering to the subject a therapeutically-effective amount of isolated AMDACs. Thus, AMDACs can be used in methods of treating diseases/disorders that would benefit from hematopoietic reconstitution.

As used herein, hematopoietic reconstitution refers to the phenomenon wherein the number and/or type of one or more cells of hematopoietic lineage, e.g., one or more hematopoietic stem cells, increase in a subject, for example, increase as a result of treatment with AMDACs relative to the number and/or type in the absence of such treatment. Without wishing to be bound by theory, increases in the number and/or type of cells of hematopoietic lineage as a result of treatment with AMDACs can result from a direct or indirect effect of the AMDACs on such cells. The phenomenon of hematopoietic reconstitution can be assessed using methods known to those of skill in the art, e.g., FACS analysis and hematological analyses, for example, red blood cell counts, hematocrit, and hemoglobin levels (see, e.g., Example 4, below).

In a specific embodiment, a subject for which hematopoietic reconstitution is indicated has been exposed to radiation (e.g., a lethal or sublethal dose of radiation). In another specific embodiment, the subject has not been exposed to radiation. In certain embodiments, the subject has undergone myeloablation, for example, myeloablation as part of cancer therapy (e.g., chemotherapy, immunotherapy) or another therapy.

In a specific embodiment, AMDACs can be used to reconstitute the hematopoietic system of a subject that has bone marrow failure or an inherited or congenital decrease in production of one or more of the major hematopoietic lineages. Disorders associated with bone marrow failure that can be treated in accordance with this embodiment include, without limitation, aplastic anemias e.g., inherited aplastic anemia (such as Fanconi's anemia, and myelodysplastic syndromes) and acquired aplastic anemias, such as anemia due to exposure to radiation, drugs, and/or chemicals (e.g., benzene). In a specific embodiment, the acquired anemia is not due to exposure to radiation.

In another specific embodiment, AMDACs can be used to reconstitute the hematopoietic system of a subject that has anemia including, but not limited to, anemia of chronic diseases such as chronic kidney disease or liver disease; autoimmune hemolytic anemia; hemoglobinopathies and thalassemias, such as sickle cell disease, or α-thalassemia or β-thalassemia.

In another specific embodiment, AMDACs can be used to reconstitute the hematopoietic system of a subject that has pure red cell aplasia, e.g., pure red cell aplasia existing as a primary disorder such as an automimmune red cell aplasia or a preleukemic red cell aplasia; or pure red cell aplasia that exists as a secondary disorder associated with a disease such as a hematologic malignancy, e.g., chronic lymphocytic leukemia, Hodgkin's disease, non-Hodgkin's lymphoma, multiple myeloma, chronic myelocytic leukemia, myelofibrosis, essential thrombocythemia or acute lymphoblastic leukemia; solid tumors, e.g., carcinoma of the stomach, adenocarcinoma of the breast or bile duct, squamous cell carcinoma of the lung, carcinoma of the thyroid, renal cell carcinoma or Kaposi's sarcoma; chronic lymphocytic anemias; drugs and chemicals, e.g., allopurinol, azathioprinie, cephalothin, estrogens, fenuprofen, halothane, isoniazid, phenobarbital, sulfathiazole or rifampicin; or severe renal failure.

In certain embodiments, said OCT-4⁻ AMDACs are HLA-G⁻, as determinable by RT-PCR. In certain other specific embodiments, said AMDACs are additionally CD49f⁺, as determinable by immunolocalization, that is, the AMDACs are OCT-4⁻, CD49f⁺. In certain other specific embodiments, said AMDACs are OCT-4⁻, HLA-G⁻ and CD49f⁺. In other specific embodiments, said AMDACs are CD90⁺, CD105⁺, or CD117⁻ as determinable by immunolocalization. In another specific embodiment, said AMDACs are CD90⁺, CD105⁺, and CD117⁻ as determinable by flow cytometry. In a more specific embodiment, said AMDACs are OCT-4⁻ and HLA-G⁻, as determined by RT-PCR, and CD49f⁺, CD90⁺, CD105⁺, and CD117⁻ as determinable by immunolocalization. In another specific embodiment, said AMDACs are VEGFR1/Flt-1⁺ (vascular endothelial growth factor receptor 1) and VEGFR2/KDR⁺ (vascular endothelial growth factor receptor 2), as determinable by immunolocalization. In another specific embodiment, said AMDACs are one or more of CD9⁺, CD10⁺, CD44⁺, CD54⁺, CD98⁺, Tie-2⁺ (angiopoietin receptor), TEM-7⁺ (tumor endothelial marker 7), CD31⁻, CD34⁻, CD45⁻, CD133⁻, CD143⁻ (angiotensin-I-converting enzyme, ACE), CD146⁻ (melanoma cell adhesion molecule), or CXCR4⁻ (chemokine (C-X-C motif) receptor 4) as determinable by immunolocalization. In another specific embodiment, said AMDACs are CD9⁺, CD10⁺, CD44⁺, CD54⁺, CD98⁺, Tie-2⁺ (angiopoietin receptor), TEM-7⁺ (tumor endothelial marker 7), CD31⁻, CD34⁻, CD45⁻, CD133⁻, CD143⁻, CD146⁻, and CXCR4⁻ as determinable by immunolocalization. In another specific embodiment of any of the above embodiments, the AMDACs are VE-cadherin⁻ as determinable by immunolocalization. In another specific embodiment, said AMDACs are additionally positive for CD105⁺ and CD200⁺ as determinable by immunolocalization. In another specific embodiment, said AMDACs do not express CD34 as determinable by immunolocalization after exposure to 50 ng/mL VEGF for 7 days.

In other specific embodiments, the AMDACs, useful to treat radiation injury, or to treat an individual having radiation injury, and/or useful in a method of hematopoietic reconstitution (e.g., useful in treating a disease that would benefit from hematopoietic reconstitution), are adherent to a tissue culture surface; wherein said AMDACs are OCT-4⁻, as determinable by RT-PCR, and CD49f⁺, HLA-G⁻, CD90⁺, CD105⁺, and CD117⁻, as determinable by immunolocalization; and wherein said AMDACs: (a) express one or more of CD9, CD10, CD44, CD54, CD98, CD200, Tie-2, TEM-7, VEGFR1/Flt-1, or VEGFR2/KDR (CD309), as determinable by immunolocalization; (b) lack expression of CD31, CD34, CD38, CD45, CD133, CD143, CD144, CD146, CD271, CXCR4, HLA-G, or VE-cadherin, as determinable by immunolocalization, or lack expression of SOX2, as determinable by RT-PCR; (c) express mRNA for ACTA2, ADAMTS1, AMOT, ANG, ANGPT1, ANGPT2, ANGPTL1, ANGPTL2, ANGPTL4, BAI1, CD44, CD200, CEACAM1, CHGA, COL15A1, COL18A1, COL4A1, COL4A2, COL4A3, CSF3, CTGF, CXCL12, CXCL2, DNMT3B, ECGF1, EDG1, EDIL3, ENPP2, EPHB2, FBLN5, F2, FGF1, FGF2, FIGF, FLT4, FN1, FST, FOXC2, GRN, HGF, HEY1, HSPG2, IFNB1, IL8, IL12A, ITGA4, ITGAV, ITGB3, MDK, MMP2, MYOZ2, NRP1, NRP2, PDGFB, PDGFRA, PDGFRB, PECAM1, PF4, PGK1, PROX1, PTN, SEMA3F, SERPINB5, SERPINC1, SERPINF1, TIMP2, TIMP3, TGFA, TGFB1, THBS1, THBS2, TIE1, TIE2/TEK, TNF, TNNI1, TNFSF15, VASH1, VEGF, VEGFB, VEGFC, VEGFR1/FLT1, or VEGFR2/KDR; (d) express one or more of the proteins CD49d, Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and II, activin receptor type IIB precursor, Wnt-9 protein, glial fibrillary acidic protein, astrocyte, myosin-binding protein C, or myosin heavy chain, nonmuscle type A; (e) secrete VEGF, HGF, IL-8, MCP-3, FGF2, Follistatin, G-CSF, EGF, ENA-78, GRO, IL-6, MCP-1, PDGF-BB, TIMP-2, uPAR, or galectin-1 into culture medium in which the AMDACs are cultured; (f) express micro RNAs miR-17-3p, miR-18a, miR-18b, miR-19b, miR-92, or miR-296 at a higher level than an equivalent number of bone marrow-derived mesenchymal stem cells; (g) express micro RNAs miR-20a, miR-20b, miR-221, miR-222, miR-15b, or miR-16 at a lower level than an equivalent number of bone marrow-derived mesenchymal stem cells; (h) express miRNAs miR-17-3p, miR-18a, miR-18b, miR-19b, miR-92, miR-20a, miR-20b, miR-296, miR-221, miR-222, miR-15b, or miR-16; and/or (i) express increased levels of CD202b, IL-8 or VEGF when cultured in less than about 5% O₂, compared to expression of CD202b, IL-8 or VEGF under 21% O₂.

The methods of treating an individual exposed to radiation, e.g., having a radiation injury, and/or treating a disease that would benefit from hematopoietic reconstitution, provided herein, may use a population of cells comprising any of the AMDACs described herein, wherein at least 50% of the cells in said population, at least 80% of the cells in said population, or at least 90% of the cells in said population are said AMDACs. In a specific embodiment, said population further comprises an isolated second type of cells, and wherein said population is not an amnion, portion of an amnion, or homogenate of an amnion. In a specific embodiment, said second type of cells are hematopoietic stem or progenitor cells, e.g., CD34⁺ cells. In other more specific embodiments, said second type of cells are embryonic stem cells, blood cells, stem cells isolated from peripheral blood, stem cells isolated from placental blood, stem cells isolated from placental perfusate, stem cells isolated from placental tissue, stem cells isolated from umbilical cord blood, umbilical cord stem cells, bone marrow-derived mesenchymal stem cells, bone marrow-derived mesenchymal stromal cells, hematopoietic stem cells, somatic stem cells, chondrocytes, fibroblasts, muscle cells, endothelial cells, angioblasts, endothelial progenitor cells, pericytes, cardiomyocytes, myocytes, cardiomyoblasts, myoblasts, or cells manipulated to resemble embryonic stem cells. In certain more specific embodiments, said second type of cells comprises at least 10%, or at least 25% of cells in said population.

The isolated amnion derived adherent cells and cell populations provided herein are not the isolated placental stem cells or cell populations described, e.g., in U.S. Pat. No. 7,255,879 or U.S. Patent Application Publication No. 2007/0275362. The isolated amnion derived adherent cells provided herein are also not endothelial progenitor cells, amniotic epithelial cells, trophoblasts, cytotrophoblasts, embryonic germ cells, embryonic stem cells, cells obtained from the inner cell mass of an embryo, or cells obtained from the gonadal ridge of an embryo.

As used herein, the term “about” means, e.g., within 10% of a stated figure or value.

As used herein, the term “stem cell” defines the functional properties of any given cell population that can proliferate extensively, but not necessarily infinitely, and contribute to the formation of multiple tissues, either during embryological development or post-natal tissue replacement and repair.

As used herein, the term “progenitor cell” defines the functional properties of any given cell population that can proliferate extensively, but not necessarily infinitely, and contribute to the formation of a restricted set of multiple tissues in comparison to a stem cell, either during embryological development or post-natal tissue replacement and repair.

As used herein, the term “derived” means isolated from or otherwise purified. For example, amnion derived adherent cells are isolated from amnion. The term “derived” encompasses cells that are cultured from cells isolated directly from a tissue, e.g., the amnion, and cells cultured or expanded from primary isolates.

As used herein, “immunolocalization” means the detection of a compound, e.g., a cellular marker, using an immune protein, e.g., an antibody or fragment thereof in, for example, flow cytometry, fluorescence-activated cell sorting, magnetic cell sorting, in situ hybridization, immunohistochemistry, or the like.

As used herein, the term “isolated cells” means cells that are substantially separated from other, cells of the tissue, e.g., amnion or placenta, from which the isolated cells are derived. Cells are “isolated” if at least about 20%, 30%, 40%, 50%, 60%, 70%, 80%, 90%, 95%, or at least 99% of the cells with which the isolated cells are naturally associated are removed from the cells, e.g., during collection and/or culture of the cells. As used herein, the term “isolated population of cells” means a population of cells that is substantially separated from other cells of the tissue, e.g., amnion, from which the population of cells is derived.

As used herein, cells are “positive” for a particular marker when that marker is detectable above background, e.g., by immunolocalization, e.g., by flow cytometry; or by RT-PCR. For example, cells are described as positive for, e.g., CD105 if CD105 is detectable on the cells in an amount detectably greater than background (in comparison to, e.g., an isotype control). In the context of, e.g., antibody-mediated detection, “positive,” as an indication a particular cell surface marker is present, means that the marker is detectable using an antibody, e.g., a fluorescently-labeled antibody, specific for that marker; “positive” also means that the cells bear that marker in a amount that produces a signal, e.g., in a flow cytometer, that is detectably above background, or above that of an isotype control. For example, cells are “CD105⁺” where the cell is detectably labeled with an antibody specific to CD105, and the signal from the antibody is detectably higher than a control (e.g., background). Conversely, “negative” in the same context means that the cell surface marker is not detectable using an antibody specific for that marker compared to background. For example, a cell is “CD34⁻” where the cell is not detectably labeled with an antibody specific to CD34. Unless otherwise noted herein, cluster of differentiation (“CD”) markers are detected using antibodies. For example, OCT-4 can be determined to be present, and a cell is OCT-4⁺, if mRNA for OCT-4 is detectable using RT-PCR, e.g., for 30 cycles.

### 4. BRIEF DESCRIPTION OF THE FIGURES

FIG. 1 shows expression of stem cell-related genes by amnion derived adherent cells and NTERA-2 cells.

FIG. 2 shows the expression of TEM-7 on the cell surface of amnion derived adherent cells (AMDACs).

FIG. 3 shows the secretion of selected angiogenic proteins by amnion derived adherent cells. 3A: secretion of TIMP1, TIMP2, thrombopoietin, VEGF, and VEGF-D. 3B: secretion of angiogenin, EGF, ENA-78, bFGF, and GRO. 3C: secretion of interferon gamma, IGF-1, IL-6, IL-8, and leptin. 3D: secretion of MCP-1, PDGF-BB, P1GF, RANTES and TGF beta 1. P6: AMDACs at passage 6. Control: no antibody. Many control values were essentially zero. Density value: output from Kodak Gel Logic 2200 Imaging System.

FIG. 4 shows the survival curves of groups of mice either exposed to radiation and administered vehicle control, exposed to radiation and administered AMDACs or Neupogen®, or administered vehicle control only.

FIG. 5 shows the survival curves of groups of mice either administered vehicle control only (Group A), exposed to radiation and administered vehicle control (Group B), or exposed to radiation and administered a specific dose of AMDACs (Groups C and D).

FIG. 6 shows the results of the comparisons of certain hematological analyses obtained for mice either administered vehicle control only, exposed to radiation and administered vehicle control, or exposed to radiation and administered a specific dose of AMDACs. P values indicate significant differences between mice exposed to radiation and treated with vehicle control (second bar from left). A: Comparison of hematocrit (HCT). B: Comparison of hemoglobin (HGB). C: Comparison of red blood cell count (RBC).

FIG. 7 provides results of FACS analyses. A: Plot of c-kit and sca-1 expression on bone marrow-derived cells from mice administered vehicle control only, exposed to radiation and administered vehicle control, or exposed to radiation and administered a specific dose of AMDACs. B: Frequency of hematopoietic stem and progenitor cells in mice exposed to radiation and administered vehicle control, or exposed to radiation and administered a specific dose of AMDACs.

### 5. DETAILED DESCRIPTION

### 5.1 Treatment of Radiation Injury

In one aspect, provided herein is a method of treating an individual who has been exposed to radiation, e.g., an individual having a radiation injury, comprising administering to the individual a therapeutically-effective amount of isolated amnion derived adherent cells (AMDACs), as described elsewhere herein, wherein said cells are adherent to a tissue culture surface, and wherein said cells are OCT-4− (POU5F1; octamer binding protein 4) as determinable by RT-PCR. The therapeutically effective amount is a number of AMDACs that results in elimination of, a detectable improvement in, lessening of the severity of, slowing of the progression of, reduction of the appearance of, or prevention of appearance of, one or more symptoms of, radiation injury. In specific embodiments, said one or more symptoms comprise one or more of nausea, vomiting, diarrhea, fever, and/or headache. In other specific embodiments, said one or more symptoms comprise purpuria, weakness, fatigue, infections, alopecia, blistering or necrosis of exposed tissue, and/or hemorrhage. In other specific embodiments, said one or more symptoms comprise neurological impairment, cognitive impairment, ataxia, tremors and/or seizures. In another specific embodiment, said one or more symptoms comprises leukopenia.

The exposure may be accidental, e.g., exposure during work in, for example, a nuclear facility, research facility or hospital, during which the exposure was unintentional, or as the result of the individual being in an area that has become contaminated with radioactive material (e.g., a zone around a nuclear blast or nuclear power plant accident). The exposure may also be caused by, adjunct to, a military action, e.g., a nuclear strike. The exposure may also be deliberate, e.g., exposure as part of remedial or clean-up activities attendant to a nuclear accident, for example, a nuclear reactor accident, or exposure as part of a medical procedure. In this embodiment, the medical procedure may be, e.g., one or more X-ray procedures involving the head, chest, thorax, abdomen, or other part of the body. The medical procedure may also be a CT scan of the head, chest, thorax, abdomen, or other part of the body. The medical procedure may also be a partial or complete radiation-induced myeloablation. In this embodiment, “partial” myeloablation means exposure to radiation of sufficient intensity and duration to kill some, but not all of the myeloid cells in the individual; “complete” myeloablation, in contrast, means exposure to radiation of sufficient intensity and duration to kill substantially all of the myeloid cells in the individual, e.g., an exposure that requires medical attention, e.g., a stem cell transplant, for example, a bone marrow transplant, in order to preserve the individual's life.

The individual need not be actually diagnosed with radiation sickness, or any symptom of radiation exposure, for treatment with AMDACs to begin; an indication that the individual has been exposed to radiation is sufficient.

The radiation injury in the individual may be caused by any kind of radiation. In certain embodiments, said radiation is ionizing radiation. In a specific embodiment, the ionizing radiation is beta radiation, gamma radiation, or X-rays. In another embodiment, said radiation is alpha radiation. In another embodiment, said radiation is neutron radiation. The individual can have experienced whole-body irradiation, e.g., in which all parts of the body receive the same, or substantially the same radiation exposure. The individual can also have experienced localized irradiation, e.g., irradiation to only a part of the individual's body.

In certain embodiments, the radiation exposure experienced by the individual, which caused the radiation injury, is acute, that is, the result of a single exposure, or exposure for a short time, e.g., less than about 1, 2, 3, 4, 5, 6, 7, 8, 9, 10, 11 or 12 hours. In certain embodiments, the acute exposure is sublethal. In other embodiments, the acute exposure is lethal, e.g., would, if not treated, cause death of the individual within 21, 20, 19, 18, 17, 16, 15, 14, 13, 12, 11, 10, 9, 8, 7, 6, 5, 4, 3, 2 or 1 days post-exposure. In certain other embodiments, the radiation exposure experienced by the individual, which caused the radiation injury, is chronic, that is, cumulative over the course of, e.g., 1-70 days or longer. For example, the individual may be exposed chronically to radiation as the result of working for an extended time in a radioactive area; living for an extended time in a radioactive area, or the like. In certain other embodiments, the exposure is substantially chronic. “Substantially chronic exposure” can include, e.g., exposure over an extended period of days, weeks, or months, during which exposure is not continuous but is chronic, e.g., exposure in a particular location as wind shifts from a radiation source. In certain embodiments, the chronic exposure is not ultimately lethal without treatment. In other embodiments, the chronic exposure is ultimately lethal without treatment.

In specific embodiments, said radiation is an acute, e.g., a single, dose of between 0.01 mSv (milliSieverts) and 0.1 mSv (0.001 rem and 0.01 rem); an acute, e.g., a single, dose of between 1 mSv and 10 mSv (between 0.1 rem and 1.0 rem) (between 0.001 Gy (Grays) and 0.01 Gy, or between 0.1 cGy (centiGrays) and 1.0 cGy); an acute, e.g., a single, dose of between 10 mSv and 100 mSv (1 rem and 10 rem) (between 0.01 Gy and 0.1 Gy, or between 1 cGy and 10 cGy); an acute, e.g., a single, dose of between 100 mSv and 1000 mSv (between 10 rem and 100 rem) (between 0.1 Gy and 1.0 Gy, or between 10 cGy and 100 cGy); a single dose of between 1000 mSv and 2000 mSv (between 100 rem and 200 rem) (between 1 Gy and 2 Gy, or between 100 cGy and 1000 cGy); an acute, e.g., a single, dose of between 2000 mSv and 3000 mSv (between 200 rem and 300 rem) (between 2 Gy and 3 Gy, or between 200 cGy and 300 cGy); an acute, e.g., a single, dose of between 3000 mSv and 4000 mSv (between 300 rem and 400 rem) (between 3 Gy and 4 Gy, or between 300 cGy and 400 cGy); an acute, e.g., a single, dose of between 4000 mSv and 5000 mSv (between 400 rem and 500 rem) (between 4 Gy and 5 Gy, or between 400 cGy and 500 cGy); or a single dose of between 5000 mSv and 10000 mSv (between 500 rem and 1000 rem) (between 5 Gy and 10 Gy, or between 500 cGy and 1000 cGy); or an acute, e.g., a single, dose of between 10000 mSv and 100000 mSv (between 1000 rem and 10000 rem) (between 10 Gy and 100 Gy, or between 1000 cGy and 10000 cGy).

In certain other embodiments, the radiation is a chronic exposure of between 0.01 mSv and 0.1 mSv (between 0.001 rem and 0.01 rem) (between 0.0001 Gy and 0.001 Gy, or between 0.01 cGy and 0.1 cGy); a chronic exposure of between 1 mSv and 10 mSv (between 0.1 rem and 1.0 rem) (0.001 Gy and 0.01 Gy, or between 0.1 cGy and 1.0 cGy); a chronic exposure of between 10 mSv and 100 mSv (between 1 rem and 10 rem) (between 0.01 Gy and 0.1 Gy, or between 1 cGy and 10 cGy); a chronic exposure of between 100 mSv and 1000 mSv (between 10 rem and 100 rem) (between 0.1 Gy and 1.0 Gy, or between 10 cGy and 100 cGy); a chronic exposure of between 1000 mSv and 2000 mSv (between 100 rem and 200 rem) (between 1 Gy and 2 Gy, or between 100 cGy and 200 cGy); a chronic exposure of between 2000 mSv and 3000 mSv (between 200 rem and 300 rem) (between 2 Gy and 3 Gy, or between 200 cGy and 300 cGy); a chronic exposure of between 3000 mSv and 4000 mSv (between 300 rem and 400 rem) (between 3 Gy and 4 Gy, or between 300 cGy and 400 cGy); a chronic exposure of between 4000 mSv and 5000 mSv (between 400 rem and 500 rem) (between 4 Gy and 5 Gy, or between 400 cGy and 500 cGy); a chronic exposure of between 5000 mSv and 10000 mSv (between 500 rem and 1000 rem) (between 5 Gy and 10 Gy, or between 500 cGy and 100 cGy); or a chronic exposure of between 10000 mSv and 100000 mSv (between 1000 rem and 10000 rem) (between 10 Gy and 100 Gy, or between 1000 cGy and 10000 cGy).

In certain specific embodiments, said chronic exposure is over 1-6 days; over 7-13 days; over 14-27 days; over 28-56 days; or over longer than 56 days. In certain other embodiment, the exposure is over 2, 3, 4, 5, 6, 7, 8, 9, 10, 11 or 12 months.

The AMDACs can be administered prophylactically, so as to ameliorate, reduce, or prevent the development of, one or more symptoms of radiation exposure, e.g., one or more symptoms of radiation sickness. Thus, in certain embodiments of the method, the individual has been exposed to the radiation, but has not yet developed one or more symptoms of acute radiation syndrome at the time of said administering. The AMDACs can also, or alternatively, be administered to the individual after one or more symptoms of radiation exposure have developed or manifested.

In specific embodiments, the individual has been exposed to said radiation in a medical setting. In a more specific embodiment, said individual has been exposed to said radiation for the purpose of myeloablation. In more specific embodiments, said myeloablation is partial (that is, at least some of the myeloid cells of the individual are allowed survive the radiation treatment, or the dose is calculated to do so) or complete (that is, the radiation exposure is designed to kill substantially all of the myeloid cells of the individual; or the radiation exposure necessitates a stem cell transplant, e.g., a bone marrow transplant, in order to preserve the life of the individual). In other specific embodiments, said individual has been exposed to radiation for another medical purpose, e.g., imaging of one or more parts of the body.

In other embodiments, the individual has been exposed in a non-medical setting, e.g., in the workplace, for example, a nuclear power facility, a research facility or a weapons facility.

In another embodiment, said individual is exposed to radiation from a source not contacting the individual's body. In another embodiment, said individual is exposed to radiation as a result of a radioactive source contacting the individual's body. In a specific embodiment, said individual is exposed to radiation as a result of the individual's inhalation or ingestion of a radioactive source, e.g., ingestion of radioactive phosphorus, sulfur, strontium, iodine, cesium, uranium, plutonium, or the like, e.g., in radioactive water, food, dust, air, or the like.

In certain specific embodiments, said administering takes place within 96 hours of said exposure; within 72 hours of said exposure; within 48 hours of said exposure; within 24 hours of said exposure; within 12 hours of exposure; within 6 hours of exposure, or within 3 hours of exposure. In certain other specific embodiments, said administering takes place within 96 hours of detection of said exposure; within 72 hours of detection of said exposure; within 48 hours of detection of said exposure; within 24 hours of detection of said exposure; within 12 hours of detection of exposure; within 6 hours of detection of exposure, or within 3 hours of detection of exposure. In certain embodiments, administration of AMDACs takes place as soon as one or more symptoms of radiation exposure, e.g., nausea, vomiting, diarrhea, headache, burning sensation in an exposed part of the body, etc. manifest itself or themselves in the exposed individual.

In certain embodiments, said individual has not yet developed one or more symptoms of acute radiation syndrome at the time of said administering. In other embodiments, said individual has developed, or is likely to develop, acute radiation syndrome or a symptom of acute radiation syndrome as a result of said exposure to radiation. In certain embodiments, the AMDACs are administered to said individual remedially; that is, after exposure, e.g., and after radiation injury, has taken place. In certain specific embodiments, said administering takes place within 96 hours of said exposure; within 72 hours of said exposure; within 48 hours of said exposure; or within 24 hours of said exposure. In certain other embodiments, the AMDACs are administered prophylactically, e.g., within about 12, 11, 10, 9, 8, 7, 6, 5, 4, 3, 2, or 1 hours prior to an expected exposure to radiation. Where exposure to radiation is anticipated, e.g., the exposure is part of a medical procedure, or is part of work in or around a contaminated area (e.g., a nuclear reactor accident), the AMDACs can be administered, once or a plurality of times, prior to exposure, e.g., 1, 2, 3, 4, 5, 6, 7, 8, 9, 10, 11 or 12 hours before said exposure.

In certain embodiments, the treatment of the individual exposed to radiation comprises a single administration of the AMDACs. In other embodiments, the treatment of the individual exposed to radiation comprises more than one, e.g., 2, 3, 4, 5, 6, 7, 8, 9, or 10 administrations of said AMDACs.

The method of treatment provided herein, in certain embodiments, comprises administration of a population of cells comprising any of the AMDACs described by the marker combinations noted above, wherein at least 50% of the cells in said population, at least 80% of the cells in said population, or at least 90% of the cells in said population are said AMDACs. The population of cells, however, is not an isolated amnion or portion thereof.

In a specific embodiment, the population of cells comprising AMDACs further comprises an isolated second type of cells, e.g., cells that may be therapeutic for radiation injury, for example, cells that may, in sufficient quantities, reconstitute the individual's hematopoietic system. In certain embodiments, for example, the second type of cells are hematopoietic stem cells, e.g., CD34+ cells, mesenchymal stem cells (e.g., bone marrow-derived mesenchymal stem cells), bone marrow-derived stromal cells, crude bone marrow, or the like. In other specific embodiments, said second type of cells are embryonic stem cells, blood cells, stem cells isolated from peripheral blood, stem cells isolated from placental blood, stem cells isolated from placental perfusate, stem cells isolated from placental tissue, stem cells isolated from umbilical cord blood, umbilical cord stem cells, somatic stem cells, chondrocytes, fibroblasts, muscle cells, endothelial cells, angioblasts, endothelial progenitor cells, pericytes, cardiomyocytes, myocytes, cardiomyoblasts, myoblasts, or cells manipulated to resemble embryonic stem cells, e.g., iPS cells. In certain more specific embodiments, said second type of cells comprises at least 10%, or at least 25% of cells in said population.

In certain embodiments, the isolated second type of cells are stem cells, e.g., tissue culture surface-adherent multipotent cells, obtained from placental tissue, e.g., the placental stem cells as described in U.S. Pat. Nos. 7,045,148; 7,255,879; and 7,311,905, and in U.S. Patent Application Publication No. 2007/0275362, the disclosures of each of which are incorporated herein by reference in their entireties. In specific embodiments, said placental stem cells are CD10⁺, CD34⁻, and CD105⁺; CD10⁺, CD34⁻, CD105⁺ and CD200⁺; CD10⁺, CD34⁻, CD45⁻, CD90⁺, CD 105⁺ and CD200⁺; or CD 10⁺, CD34⁻, CD45⁻, CD80⁻, CD86⁻, CD90⁺, CD 105⁺ and CD200⁺. In other specific embodiments, said placental stem cells are CD200⁺ and HLA-G⁺; CD73⁺, CD 105⁺, and CD200⁺; CD200⁺ and OCT-4⁺; CD73⁺, CD 105⁺ and HLA-G⁺; CD73⁺ and CD105⁺ and facilitate the formation of one or more embryoid-like bodies in a population of placental cells comprising said stem cell when said population is cultured under conditions that allow the formation of an embryoid-like body; or OCT-4⁺ and facilitate the formation of one or more embryoid-like bodies in a population of placental cells comprising the stem cell when said population is cultured under conditions that allow formation of embryoid-like bodies; or any combination thereof. In a more specific embodiment, said CD200⁺, HLA-G⁺ stem cells are CD34⁻, CD38⁻, CD45⁻, CD73⁺ and CD105⁺. In another more specific embodiment, said CD73⁺, CD105⁺, and CD200⁺ stem cells are CD34⁻, CD38⁻, CD45⁻, and HLA-G⁺. In another more specific embodiment, said CD200⁺, OCT-4⁺ stem cells are CD34⁻, CD38⁻, CD45⁻, CD73⁺, CD105⁺ and HLA-G⁺. In another more specific embodiment, said CD73⁺, CD105⁺ and HLA-G⁺ stem cells are CD34⁻, CD45⁻, OCT-4⁺ and CD200⁺. In another more specific embodiment, said CD73⁺ and CD105⁺ stem cells are OCT-4⁺, CD34⁻, CD38⁻ and CD45⁻. In another more specific embodiment, said OCT-4⁺ stem cells are CD73⁺, CD105⁺, CD200⁺, CD34⁻, CD38⁻, and CD45⁻. In another more specific embodiment, the placental stem cells are maternal in origin (that is, have the maternal genotype). In another more specific embodiment, the placental stem cells are fetal in origin (that is, have the fetal genotype).

AMDACs can be combined with a plurality of cells of another type, e.g., with a population of stem cells, in a ratio of about 100,000,000:1, 50,000,000:1, 20,000,000:1, 10,000,000:1, 5,000,000:1, 2,000,000:1, 1,000,000:1, 500,000:1, 200,000:1, 100,000:1, 50,000:1, 20,000:1, 10,000:1, 5,000:1, 2,000:1, 1,000:1, 500:1, 200:1, 100:1, 50:1, 20:1, 10:1, 5:1, 2:1, 1:1; 1:2; 1:5; 1:10; 1:100; 1:200; 1:500; 1:1,000; 1:2,000; 1:5,000; 1:10,000; 1:20,000; 1:50,000; 1:100,000; 1:500,000; 1:1,000,000; 1:2,000,000; 1:5,000,000; 1:10,000,000; 1:20,000,000; 1:50,000,000; or about 1:100,000,000, comparing numbers of total nucleated cells in each population.

### 5.2 Hematopoietic Reconstitution

In another aspect, provided herein is a method of inducing hematopoietic reconstitution (e.g., partial or complete hematopoietic reconstitution) in a subject in need thereof, e.g., in a subject that has suffered a partial or total loss of hematopoietic stem cells, comprising administering to the subject a therapeutically-effective amount of isolated AMDACs. Thus, AMDACs can be used in methods of treating diseases/disorders that would benefit from hematopoietic reconstitution.

As used herein, hematopoietic reconstitution refers to the phenomenon wherein the number and/or type of one or more cells of hematopoietic lineage, e.g., one or more hematopoietic stem cells, increase in a subject, for example, increase as a result of treatment with AMDACs relative to the number and/or type in the absence of such treatment. Without wishing to be bound by theory, increases in the number and/or type of cells of hematopoietic lineage as a result of treatment with AMDACs can result from a direct or indirect effect of the AMDACs on such cells. The phenomenon of hematopoietic reconstitution can be assessed using methods known to those of skill in the art, e.g., FACS analysis and hematological analyses, for example, red blood cell counts, hematocrit, and hemoglobin levels (see, e.g., Example 4, below).

In a specific embodiment, a subject that has suffered a partial or total loss of hematopoietic stem cells for which hematopoietic reconstitution is indicated has been exposed to radiation (e.g., a lethal or sublethal dose of radiation). In another specific embodiment, the subject has not been exposed to radiation. In certain embodiments, the subject has undergone myeloablation, for example, myeloablation as part of cancer therapy (e.g., chemotherapy, immunotherapy) or another therapy.

In a specific embodiment, AMDACs can be used to reconstitute the hematopoietic system of a subject that has bone marrow failure or an inherited or congenital decrease in production of one or more of the major hematopoietic lineages. Disorders associated with bone marrow failure that can be treated in accordance with this embodiment include, without limitation, aplastic anemias e.g., inherited aplastic anemia (such as Fanconi's anemia, and myelodysplastic syndromes) and acquired aplastic anemias, such as anemia due to exposure to radiation, drugs, and/or chemicals (e.g., benzene). In a specific embodiment, the acquired anemia is not due to exposure to radiation.

In another specific embodiment, AMDACs can be used to reconstitute the hematopoietic system of a subject that has anemia including, but not limited to, anemia of chronic diseases such as chronic kidney disease or liver disease; autoimmune hemolytic anemia; hemoglobinopathies and thalassemias, such as sickle cell disease, or α-thalassemia or β-thalassemia.

In another specific embodiment, AMDACs can be used to reconstitute the hematopoietic system of a subject that has pure red cell aplasia, e.g., pure red cell aplasia existing as a primary disorder such as an automimmune red cell aplasia or a preleukemic red cell aplasia; or pure red cell aplasia that exists as a secondary disorder associated with a disease such as a hematologic malignancy, e.g., chronic lymphocytic leukemia, Hodgkin's disease, non-Hodgkin's lymphoma, multiple myeloma, chronic myelocytic leukemia, myelofibrosis, essential thrombocythemia or acute lymphoblastic leukemia; solid tumors, e.g., carcinoma of the stomach, adenocarcinoma of the breast or bile duct, squamous cell carcinoma of the lung, carcinoma of the thyroid, renal cell carcinoma or Kaposi's sarcoma; chronic lymphocytic anemias; drugs and chemicals, e.g., allopurinol, azathioprinie, cephalothin, estrogens, fenuprofen, halothane, isoniazid, phenobarbital, sulfathiazole or rifampicin; or severe renal failure.

In certain embodiments, hematopoietic reconstitution in a subject that has been exposed to a condition (e.g., radiation or myeloablation) that causes a reduction in the number and/or type of cells of hematopoietic lineage in the subject refers to an increase in the number and/or type of cells of hematopoietic lineage in the subject relative to the number and/or type of such cells in the subject prior to treatment with AMDACs and/or the number and/or type of such cells that would be expected to be found in the subject if the subject were not exposed to the condition that caused a reduction in the number of cells of hematopoietic lineage. In certain embodiments, hematopoietic reconstitution in a subject suffering from a disease or disorder that would benefit from hematopoietic reconstitution refers to an increase in the number and/or type of cells of hematopoietic lineage in the subject relative to the number and/or type of such cells in the subject prior to treatment with AMDACs and/or the number and/or type of such cells that would be expected to be found in the subject if the subject were not suffering from the disease or disorder that causes a reduction in the number of cells of hematopoietic lineage.

### 5.3 Characteristics of Amnion Derived Adherent Cells

The AMDACs, useful in the methods of treating radiation injury and hematopoietic reconstitution provided herein, are obtainable from the amniotic membrane by a two-step isolation procedure described below, adhere to a cell culture surface, e.g., to tissue culture plastic, are OCT-4⁻ (octamer binding protein 4), as determinable by RT-PCR, and display some or all of the characteristics listed below.

AMDACs display cellular markers that distinguish them from other amnion-derived, or placenta-derived, cells. For example, in one embodiment, the OCT-4⁻ AMDACs are additionally CD49f⁺, as determinable by immunolocalization. In another specific embodiment, said AMDACs are HLA-G⁻, as determined by RT-PCR. In another specific embodiment, the OCT-4⁻ AMDACs are VEGFR1/Flt-1⁺ (vascular endothelial growth factor receptor 1) and/or VEGFR2/KDR⁺ (vascular endothelial growth factor receptor 2), as determinable by immunolocalization. In a specific embodiment, the OCT-4⁻ AMDACs express at least 2 log less PCR-amplified mRNA for OCT-4 at, e.g., 20 cycles, than an equivalent number of NTERA-2 cells for an equivalent number of RNA amplification cycles. In another specific embodiment, said OCT-4⁻ AMDACs are CD90⁺, CD105⁺, or CD117⁻. In a more specific embodiment, said OCT-4⁻ AMDACs are CD90⁺, CD105⁺, and CD117⁻, e.g., as determinable by immunolocalization. In a more specific embodiment, the AMDACs are OCT-4⁻ and/or HLA-G⁻, and are additionally CD49f⁺, CD90⁺, CD105⁺, and CD117⁻, e.g., as determinable by immunolocalization. In a more specific embodiment, the AMDACs are OCT-4⁻, HLA-G⁻, CD49f⁺, CD90⁺, CD105⁺, and CD117⁻, e.g., as determinable by immunolocalization. In another specific embodiment, the OCT-4⁻ AMDACs do not express SOX2, e.g., as determinable by RT-PCR for 30 cycles. In a specific embodiment, therefore, the cell is OCT-4⁻, CD49f⁺, CD90⁺, CD105⁺, and CD117⁻, as determinable by immunolocalization, and SOX2⁻, as determinable by RT-PCR, e.g., for 30 cycles.

In another embodiment, said OCT-4⁻ AMDACs are one or more of CD29⁺, CD73⁺, ABC-p⁺, and CD38⁻, as determined by immunolocalization.

In another specific embodiment, for example, the OCT-4⁻ AMDACs are additionally one or more of CD9⁺, CD10⁺, CD44⁺, CD54⁺, CD98⁺, TEM-7⁺ (tumor endothelial marker 7), CD31⁻, CD34⁻, CD45⁻, CD133⁻, CD143⁻ (angiotensin-I-converting enzyme, ACE), CD146⁻ (melanoma cell adhesion molecule), or CXCR4⁻ (chemokine (C-X-C motif) receptor 4) as determined by immunolocalization, or HLA-G⁻ as determined by RT-PCR. In a more specific embodiment, said cell is CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, and CXCR4⁻ as determined by immunolocalization, and HLA-G⁻ as determined by RT-PCR. In one embodiment, the amnion derived adherent cell provided herein is one or more of CD31⁻, CD34⁻, CD45⁻, and/or CD133⁻. In a specific embodiment, the amnion derived adherent cell is OCT-4⁻, as determined by RT-PCR; VEGFR1/Flt-1⁺ and/or VEGFR2/KDR⁺, as determined by immunolocalization; and one or more, or all, of CD31⁻, CD34⁻, CD45⁻, and/or CD133⁻.

In another specific embodiment, said cell is additionally VE-cadherin⁻ as determined by immunolocalization. In another specific embodiment, said cell is additionally positive for CD105⁺ and CD200⁺ as determined by immunolocalization. In another specific embodiment, said cell does not express CD34 as detected by immunolocalization after exposure to 1 to 100 ng/mL VEGF for 4 to 21 days. In more specific embodiments, said cell does not express CD34 as detected by immunolocalization after exposure to 25 to 75 ng/mL VEGF for 4 to 21 days, or to 50 ng/mL VEGF for 4 to 21 days. In even more specific embodiments, said cell does not express CD34 as detected by immunolocalization after exposure to 1, 2.5, 5, 10, 25, 50, 75 or 100 ng/mL VEGF for 4 to 21 days. In yet more specific embodiments, said cell does not express CD34 as detected by immunolocalization after exposure to 1 to 100 ng/mL VEGF for 7 to 14, e.g., 7, days.

In specific embodiments, the amnion derived adherent cell is OCT-4⁻, as determined by RT-PCR, and one or more of VE-cadherin⁻, VEGFR2/KDR⁺, CD9⁺, CD54⁺, CD105⁺, and/or CD200⁺ as determined by immunolocalization. In a specific embodiment, the amnion derived cell is OCT-4⁻, as determined by RT-PCR, and VE-cadherin⁻, VEGFR2/KDR⁺, CD9⁺, CD54⁺, CD105⁺, and CD200⁺ as determined by immunolocalization. In another specific embodiment, said cells do not express CD34, as detected by immunolocalization, e.g., after exposure to 1 to 100 ng/mL VEGF for 4 to 21 days.

In another embodiment, the amnion derived adherent cell is OCT-4⁻, CD49f⁺, HLA-G⁻, CD90⁺, CD105⁺, and CD117⁻. In a more specific embodiment, said cell is one or more of CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, or CXCR4⁻, as determined by immunolocalization. In a more specific embodiment, said cell is CD9⁺, CD10⁺, CD44⁺, CD54⁺, CD98⁺, Tie-2⁺, TEM-7⁺, CD31⁻, CD34⁻, CD45⁻, CD133⁻, CD143⁻, CD146⁻, and CXCR4⁻ as determined by immunolocalization. In another specific embodiment, said cell is additionally VEGFR1/Flt-1⁺ and/or VEGFR2/KDR⁺, as determined by immunolocalization; and one or more of CD31⁻, CD34⁻, CD45⁻, CD133⁻, and/or Tie-2⁻ as determined by immunolocalization. In another specific embodiment, said cell is additionally VEGFR1/Flt-1⁺, VEGFR2/KDR⁺, CD31⁻, CD34⁻, CD45⁻, CD133⁻, and Tie-2⁻ as determined by immunolocalization.

In another embodiment, the OCT-4− amnion derived adherent cells are additionally one or more, or all, of CD9⁺, CD10⁺, CD44⁺, CD49f⁺, CD54⁺, CD90⁺, CD98⁺, CD105⁺, CD200⁺, Tie-2⁺, TEM-7⁺, VEGFR1/Flt-1⁺, and/or VEGFR2/KDR⁺ (CD309⁺), as determined by immunolocalization; or additionally one or more, or all, of CD31⁻, CD34⁻, CD38⁻, CD45⁻, CD117⁻, CD133⁻, CD143⁻, CD144⁻, CD146⁻, CD271⁻, CXCR4⁻, HLA-G⁻, and/or VE-cadherin⁻, as determined by immunolocalization, or SOX2⁻, as determined by RT-PCR.

In certain embodiments, the isolated tissue culture plastic-adherent amnion derived adherent cells are CD49f⁺. In a specific embodiment, said CD49f⁺ cells are additionally one or more, or all, of CD9⁺, CD10⁺, CD44⁺, CD54⁺, CD90⁺, CD98⁺, CD105⁺, CD200⁺, Tie-2⁺, TEM-7⁺, VEGFR1/Flt-1⁺, and/or VEGFR2/KDR⁺ (CD309⁺), as determined by immunolocalization; or additionally one or more, or all, of CD31⁻, CD34⁻, CD38⁻, CD45⁻, CD117⁻, CD133⁻, CD143⁻, CD144⁻, CD146⁻, CD271⁻, CXCR4⁻, HLA-G⁻, OCT-4⁻ and/or VE-cadherin⁻, as determined by immunolocalization, or SOX2⁻, as determined by RT-PCR.

In certain other embodiments, the isolated tissue culture plastic-adherent amnion derived adherent cells are HLA-G⁻, CD90⁺, and CD117⁻. In a specific embodiment, said HLA-G⁻, CD90⁺, and CD117⁻ cells are additionally one or more, or all, of CD9⁺, CD10⁺, CD44⁺, CD49f⁺, CD54⁺, CD98⁺, CD105⁺, CD200⁺, Tie-2⁺, TEM-7⁺, VEGFR1/Flt-1⁺, and/or VEGFR2/KDR⁺ (CD309⁺), as determined by immunolocalization; or additionally one or more, or all, of CD31⁻, CD34⁻, CD38⁻, CD45⁻, CD133⁻, CD143⁻, CD144⁻, CD146⁻, CD271⁻, CXCR4⁻, OCT-4⁻ and/or VE-cadherin⁻, as determined by immunolocalization, or SOX2⁻, as determined by RT-PCR.

In another embodiment, the isolated amnion derived adherent cells, or population of amnion derived angiogenic cells, do not constitutively express mRNA for fibroblast growth factor 4 (FGF4), interferon γ (IFNG), chemokine (C-X-C motif) ligand 10 (CXCL10), angiopoietin 4 (ANGPT4), angiopoietin-like 3 (ANGPTL3), fibrinogen α chain (FGA), leptin (LEP), prolactin (PRL), prokineticin 1 (PROK1), tenomodulin (TNMD), FMS-like tyrosine kinase 3 (FLT3), extracellular link domain containing 1 (XLKD1), cadherin 5, type 2 (CDH5), leukocyte cell derived chemotaxin 1 (LECT1), plasminogen (PLG), telomerase reverse transcriptase (TERT), (sex determining region Y)-box 2 (SOX2), NANOG, matrix metalloprotease 13 (MMP-13), distal-less homeobox 5 (DLX5), and/or bone gamma-carboxyglutamate (gla) protein (BGLAP), as determined by RT-PCR, e.g., for 30 cycles under standard culture conditions. In other embodiments, isolated amnion derived adherent cells, or population of amnion derived angiogenic cells, express mRNA for (ARNT2), nerve growth factor (NGF), brain-derived neurotrophic factor (BDNF), glial-derived neurotrophic factor (GDNF), neurotrophin 3 (NT-3), NT-5, hypoxia-Inducible Factor 1α (HIF1A), hypoxia-inducible protein 2 (HIG2), heme oxygenase (decycling) 1 (HMOX1), Extracellular superoxide dismutase [Cu—Zn] (SOD3), catalase (CAT), transforming growth factor β1 (TGFB1), transforming growth factor β1 receptor (TGFB 1R), and hepatoycte growth factor receptor (HGFR/c-met)

In another aspect, provided herein are isolated populations of cells comprising the amnion derived adherent cells described herein. The populations of cells can be homogeneous populations, e.g., a population of cells, at least about 90%, 95%, 98% or 99% of which are amnion derived adherent cells. The populations of cells can be heterogeneous, e.g., a population of cells wherein at most about 10%, 20%, 30%, 40%, 50%, 60%, 70% or 80% of the cells in the population are amnion derived adherent cells. The isolated populations of cells are not, however, tissue, i.e., amniotic membrane.

In one embodiment, provided herein is an isolated population of cells comprising AMDACs, e.g., a population of cells substantially homogeneous for AMDACs, wherein said AMDACs are adherent to tissue culture plastic, and wherein said AMDACs are OCT-4⁻, as determined by RT-PCR. In a specific embodiment, the AMDACs are CD49f⁺ or HLA-G⁺, e.g., as determined by immunolocalization or RT-PCR. In another specific embodiment, said population of AMDACs is VEGFR1/Flt-1⁺ and/or VEGFR2/KDR⁺ as determined by immunolocalization, wherein said isolated population of cells is not an amnion or amniotic membrane. In a more specific embodiment, the AMDACs are OCT-4⁻, and/or HLA-G⁻ as determined by RT-PCR, and VEGFR1/Flt-1⁺ and/or VEGFR2/KDR⁺ as determined by immunolocalization. In a specific embodiment, at least about 50%, 60%, 70%, 80%, 90%, 95%, 98% or 99% of cells in said population are said amnion derived adherent cells. In another specific embodiment, said AMDACs are CD90⁺, CD105⁺, or CD117⁻. In a more specific embodiment, said AMDACs are CD90⁺, CD105⁺, and CD117⁻. In a more specific embodiment, the AMDACs are OCT-4⁻, CD49f⁺, CD90⁺, CD105⁺, and CD117⁻. In another specific embodiment, the AMDACs do not express SOX2, e.g., as determined by RT-PCR for 30 cycles. In an even more specific embodiment, the population comprises AMDACs, wherein said AMDACs are OCT-4⁻, HLA-G⁻, CD49f⁺, CD90⁺, CD105⁺, and CD117⁻, as determined by immunolocalization or flow cytometry, and SOX2⁻, e.g., as determined by RT-PCR for 30 cycles

In another specific embodiment, said AMDACs in said population of cells are CD90⁺, CD105⁺, or CD117⁻, as determined by immunolocalization or flow cytometry. In a more specific embodiment, the AMDACs are CD90⁺, CD105⁺, and CD117⁻, as determined by immunolocalization or flow cytometry. In a more specific embodiment, the AMDACs are OCT-4⁻ or HLA-G⁻, e.g., as determined by RT-PCR, and are additionally CD49f⁺, CD90⁺, CD105⁺, and CD117⁻ as determined by immunolocalization or flow cytometry. In a more specific embodiment, the AMDACs in said population of cells are OCT-4⁻, HLA-G⁻, CD49f⁺, CD90⁺, CD105⁺, and CD117⁻. In another specific embodiment, the AMDACs do not express SOX2, e.g., as determined by RT-PCR for 30 cycles. In a more specific embodiment, therefore, the cell is OCT-4⁻, CD49f⁺, CD90⁺, CD105⁺, and CD117⁻, as determined by immunolocalization or flow cytometry, and SOX2⁻, as determined by RT-PCR, e.g., for 30 cycles. In an even more specific embodiment, the AMDACs are OCT-4⁻ or HLA-G⁻, and are additionally CD49f⁺, CD90⁺, CD105⁺, and CD117⁻. In a more specific embodiment, the AMDACs are OCT-4⁻, HLA-G⁻, CD49f⁺, CD90⁺, CD105⁺, and CD117⁻.

In another embodiment, the amnion derived adherent cells in said population of cells are adherent to tissue culture plastic, OCT-4⁻ as determined by RT-PCR, and VEGFR1/Flt-1⁺ and/or VEGFR2/KDR⁺ as determined by immunolocalization, and are additionally one or more of CD9⁺, CD10⁺, CD44⁺, CD54⁺, CD98⁺, Tie-2⁺, TEM-7⁺, CD31⁻, CD34⁻, CD45⁻, CD133⁻, CD 143⁻, CD 146⁻, or CXCR4⁻, as determined by immunolocalization, or HLA-G⁻ as determined by RT-PCR, and wherein said isolated population of cells is not an amnion. In another embodiment, provided herein is an isolated population of cells comprising an amnion derived adherent cell, wherein said cell is adherent to tissue culture plastic, wherein said cell is OCT-4⁻ as determined by RT-PCR, and VEGFR1/Flt-1⁺ and/or VEGFR2/KDR⁺ as determined by immunolocalization, wherein said cell does not express CD34 as detected by immunolocalization after exposure to 1 to 100 ng/mL VEGF for 4 to 21 days, and wherein said isolated population of cells is not an amnion. In a specific embodiment of any of the above embodiments, at least about 50%, 60%, 70%, 80%, 90%, 95%, 98% or 99% of cells in said population are said amnion derived adherent cells.

In another embodiment, any of the above populations of cells comprising amnion derived adherent cells forms sprouts or tube-like structures when cultured in the presence of an extracellular matrix protein, e.g., like collagen type I and IV, or an angiogenic factor, e.g., like vascular endothelial growth factor (VEGF), epithelial growth factor (EGF), platelet derived growth factor (PDGF) or basic fibroblast growth factor (bFGF), e.g., in or on a substrate such as placental collagen, e.g., or MATRIGEL™ for at least 4 days and up to 14 days.

Amnion derived adherent cells, and populations of amnion derived adherent cells, display characteristic expression of proteins related to angiogenesis-related or cardiomyogenesis-related genes. In certain embodiments, provided herein is a cell that expresses, or a population of cells, wherein at least about 50%, 60%, 70%, 80%, 90%, 95% or 98% of cells in said isolated population of cells are amnion derived adherent cells that express RNA for one or more of, or all of, ACTA2 (actin, alpha 2, smooth muscle, aorta), ADAMTS1 (ADAM metallopeptidase with thrombospondin type 1 motif, 1), AMOT (angiomotin), ANG (angiogenin), ANGPT1 (angiopoietin 1), ANGPT2, ANGPTL1 (angiopoietin-like 1), ANGPTL2, ANGPTL4, BAI1 (brain-specific angiogenesis inhibitor 1), CD44, CD200, CEACAM1 (carcinoembryonic antigen-related cell adhesion molecule 1), CHGA (chromogranin A), COL15A1 (collagen, type XV, alpha 1), COL18A1 (collagen, type XVIII, alpha 1), COL4A1 (collagen, type IV, alpha 1), COL4A2 (collagen, type IV, alpha 2), COL4A3 (collagen, type IV, alpha 3), CSF3 (colony stimulating factor 3 (granulocyte), CTGF (connective tissue growth factor), CXCL12 (chemokine (CXC motif) ligand 12 (stromal cell-derived factor 1)), CXCL2, DNMT3B (DNA (cytosine-5-)-methyltransferase 3 beta), ECGF1 (thymidine phosphorylase), EDG1 (endothelial cell differentiation gene 1), EDIL3 (EGF-like repeats and discoidin I-like domains 3), ENPP2 (ectonucleotide pyrophosphatase/phosphodiesterase 2), EPHB2 (EPH receptor B2), FBLN5 (FIBULIN 5), F2 (coagulation factor II (thrombin)), FGF1 (acidic fibroblast growth factor), FGF2 (basic fibroblast growth factor), FIGF (c-fos induced growth factor (vascular endothelial growth factor D)), FLT4 (fms-related tyrosine kinase 4), FN1 (fibronectin 1), FST (follistatin), FOXC2 (forkhead box C2 (MFH-1, mesenchyme forkhead 1)), GRN (granulin), HGF (hepatocyte growth factor), HEY1 (hairy/enhancer-of-split related with YRPW motif 1), HSPG2 (heparan sulfate proteoglycan 2), IFNB1 (interferon, beta 1, fibroblast), IL8 (interleukin 8), IL12A, ITGA4 (integrin, alpha 4; CD49d), ITGAV (integrin, alpha V), ITGB3 (integrin, beta 3), MDK (midkine), MMP2 (matrix metalloprotease 2), MYOZ2 (myozenin 2), NRP1 (neuropilin 1), NRP2, PDGFB (platelet-derived growth factor β), PDGFRA (platelet-derived growth factor receptor a), PDGFRB, PECAM1 (platelet/endothelial cell adhesion molecule), PF4 (platelet factor 4), PGK1 (phosphoglycerate kinase 1), PROX1 (prospero homeobox 1), PTN (pleiotrophin), SEMA3F (semophorin 3F), SERPINB5 (serpin peptidase inhibitor, Glade B (ovalbumin), member 5), SERPINC1, SERPINF1, TIMP2 (tissue inhibitor of metalloproteinases 2), TIMP3, TGFA (transforming growth factor, alpha), TGFB1, THBS1 (thrombospondin 1), THBS2, TIE1 (tyrosine kinase with immunoglobulin-like and EGF-like domains 1), TIE2/TEK, TNF (tumor necrosis factor), TNNI1 (troponin I, type 1), TNFSF15 (tumor necrosis factor (ligand) superfamily, member 15), VASH1 (vasohibin 1), VEGF (vascular endothelial growth factor), VEGFB, VEGFC, VEGFR1/FLT1 (vascular endothelial growth factor receptor 1), and/or VEGFR2/KDR.

When human cells are used, the gene designations throughout refer to human sequences, and, as is well known to persons of skill in the art, representative sequences can be found in literature, or in GenBank. Probes to the sequences can be determined by sequences that are publicly-available, or through commercial sources, e.g., specific TAQMAN® probes or TAQMAN® Angiogenesis Array (Applied Biosystems, part no. 4378710).

Amnion derived adherent cells, and populations of amnion derived adherent cells, display characteristic expression of angiogenesis-related proteins. In certain embodiments, provided herein is a cell that expresses, or a population of cells, wherein at least about 50%, 60%, 70%, 80%, 90%, 95% or 98% of cells in said isolated population of cells are amnion derived adherent cells that express CD49d, Connexin-43, HLA-ABC, Beta 2-microglobulin, CD349, CD318, PDL1, CD106, Galectin-1, ADAM 17 precursor (A disintegrin and metalloproteinase domain 17) (TNF-alpha converting enzyme) (TNF-alpha convertase), Angiotensinogen precursor, Filamin A (Alpha-filamin) (Filamin 1) (Endothelial actin-binding protein) (ABP-280) (Nonmuscle filamin), Alpha-actinin 1 (Alpha-actinin cytoskeletal isoform) (Non-muscle alpha-actinin 1) (F-actin cross linking protein), Low-density lipoprotein receptor-related protein 2 precursor (Megalin) (Glycoprotein 330) (gp330), Macrophage scavenger receptor types I and II (Macrophage acetylated LDL receptor I and II), Activin receptor type JIB precursor (ACTR-IIB), Wnt-9 protein, Glial fibrillary acidic protein, astrocyte (GFAP), Myosin-binding protein C, cardiac-type (Cardiac MyBP-C) (C-protein, cardiac muscle isoform), and/or Myosin heavy chain, nonmuscle type A (Cellular myosin heavy chain, type A) (Nonmuscle myosin heavy chain-A) (NMMHC-A).

The amnion derived adherent cells provided herein further secrete proteins that promote angiogenesis, e.g., in endothelial cells, endothelial progenitor cells, or the like. In certain embodiments, the amnion derived adherent cell, population of amnion derived adherent cells, or population of cells comprising amnion derived adherent cells, e.g., wherein at least about 50%, 60%, 70%, 80%, 90%, 95% or 98% of cells in said isolated population of cells are amnion derived adherent cells, secrete one or more, or all, of VEGF, HGF, IL-8, MCP-3, FGF2, Follistatin, G-CSF, EGF, ENA-78, GRO, IL-6, MCP-1, PDGF-BB, TIMP-2, uPAR, Galectin-1, e.g., into culture medium in which the cell, or cells, are grown.

In another embodiment, any of the above populations of cells comprising amnion derived adherent cells can cause the formation of sprouts or tube-like structures in a population of endothelial cells in contact with said amnion derived adherent cells. In a specific embodiment, the amnion-derived angiogenic cells are co-cultured with human endothelial cells, forming sprouts or tube-like structures, or supporting the endothelial cell sprouts, e.g., when cultured in the presence of extracellular matrix proteins such as collagen type I and IV, and/or angiogenic factors such as vascular endothelial growth factor (VEGF), epithelial growth factor (EGF), platelet derived growth factor (PDGF) or basic fibroblast growth factor (bFGF), e.g., in or on a substrate such as placental collagen or MATRIGEL™ for at least 4 days and/or up to 14 days.

In another embodiment, any of the above populations of cells comprising amnion derived adherent cells secrete angiogenic factors such as vascular endothelial growth factor (VEGF), epithelial growth factor (EGF), platelet derived growth factor (PDGF), basic fibroblast growth factor (bFGF), or Interleukin-8 (IL-8) and thereby can induce human endothelial cells to form sprouts or tube-like structures when cultured in the presence of extracellular matrix proteins such as collagen type I and IV e.g., in or on a substrate such as placental collagen or MATRIGEL™.

In another embodiment, provided herein is a population of cells, e.g., a population of amnion derived adherent cells, or a population of cells wherein at least about 50%, 60%, 70%, 80%, 90%, 95% or 98% of cells in said isolated population of cells are amnion derived adherent cells that express angiogenic micro RNAs (miRNAs) at a higher level than bone marrow-derived mesenchymal stem cells, wherein said miRNAs comprise one or more, or all of, miR-17-3p, miR-18a, miR-18b, miR-19b, miR-92, and/or miR-296. In another embodiment, provided herein is a population of cells, e.g., a population of amnion derived adherent cells, or a population of cells wherein at least about 50%, 60%, 70%, 80%, 90%, 95% or 98% of cells in said isolated population of cells are amnion derived adherent cells that express one or more of, or all of, angiogenic micro RNAs (miRNAs) at a lower level than bone marrow-derived mesenchymal stem cells, wherein said miRNAs comprise one or more, or all of, miR-20a, miR-20b, miR-221, miR-222, miR-15b, and/or miR-16. In certain embodiments, AMDACs, or populations of AMDACs, express one or more, or all, of the angiogenic miRNAs miR-17-3p, miR-18a, miR-18b, miR-19b, miR-92, miR-20a, miR-20b, (members of the of the angiogenic miRNA cluster 17-92), miR-296, miR-221, miR-222, miR-15b, and/or miR-16.

Thus, in one embodiment, provided herein is an isolated amnion derived adherent cell, wherein said cell is adherent to tissue culture plastic, and wherein said cell is OCT-4⁻, as determined by RT-PCR, and CD49f⁺, HLA-G⁻, CD90⁺, CD105⁺, and CD117⁻, as determined by immunolocalization, and wherein said cell: (a) expresses one or more of CD9, CD10, CD44, CD54, CD98, CD200, Tie-2, TEM-7, VEGFR1/Flt-1, or VEGFR2/KDR (CD309), as determined by immunolocalization; (b) lacks expression of CD31, CD34, CD38, CD45, CD133, CD143, CD144, CD146, CD271, CXCR4, HLA-G, or VE-cadherin, as determined by immunolocalization, or lacks expression of SOX2, as determined by RT-PCR; (c) express mRNA for ACTA2, ADAMTS1, AMOT, ANG, ANGPT1, ANGPT2, ANGPTL1, ANGPTL2, ANGPTL4, BAI1, CD44, CD200, CEACAM1, CHGA, COL15A1, COL18A1, COL4A1, COL4A2, COL4A3, CSF3, CTGF, CXCL12, CXCL2, DNMT3B, ECGF1, EDG1, EDIL3, ENPP2, EPHB2, FBLN5, F2, FGF1, FGF2, FIGF, FLT4, FN1, FST, FOXC2, GRN, HGF, HEY1, HSPG2, IFNB1, IL8, IL12A, ITGA4, ITGAV, ITGB3, MDK, MMP2, MYOZ2, NRP1, NRP2, PDGFB, PDGFRA, PDGFRB, PECAM1, PF4, PGK1, PROX1, PTN, SEMA3F, SERPINB5, SERPINC1, SERPINF1, TIMP2, TIMP3, TGFA, TGFB1, THBS1, THBS2, TIE1, TIE2/TEK, TNF, TNNI1, TNFSF15, VASH1, VEGF, VEGFB, VEGFC, VEGFR1/FLT1, or VEGFR2/KDR; (d) expresses one or more of the proteins CD49d, Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and II, activin receptor type IIB precursor, Wnt-9 protein, glial fibrillary acidic protein, astrocyte, myosin-binding protein C, or myosin heavy chain, nonmuscle type A; (e) secretes VEGF, HGF, IL-8, MCP-3, FGF2, Follistatin, G-CSF, EGF, ENA-78, GRO, IL-6, MCP-1, PDGF-BB, TIMP-2, uPAR, or galectin-1 into culture medium in which the cell grows; (f) expresses micro RNAs miR-17-3p, miR-18a, miR-18b, miR-19b, miR-92, or miR-296 at a higher level than an equivalent number of bone marrow-derived mesenchymal stem cells; (g) expresses micro RNAs miR-20a, miR-20b, miR-221, miR-222, miR-15b, or miR-16 at a lower level than an equivalent number of bone marrow-derived mesenchymal stem cells; (h) expresses miRNAs miR-17-3p, miR-18a, miR-18b, miR-19b, miR-92, miR-20a, miR-20b, miR-296, miR-221, miR-222, miR-15b, or miR-16; and/or (i) expresses increased levels of CD202b, IL-8 or VEGF when cultured in less than about 5% O₂, compared to expression of CD202b, IL-8 or VEGF under 21% O₂. In a specific embodiment, the isolated amnion derived adherent cell is OCT-4⁻, as determined by RT-PCR, and CD49f⁺, HLA-G⁻, CD90⁺, CD105⁺, and CD117⁻, as determined by immunolocalization, and (a) expresses CD9, CD10, CD44, CD54, CD90, CD98, CD200, Tie-2, TEM-7, VEGFR1/Flt-1, and/or VEGFR2/KDR (CD309), as determined by immunolocalization; (b) lacks expression of CD31, CD34, CD38, CD45, CD133, CD143, CD144, CD146, CD271, CXCR4, HLA-G, and/or VE-cadherin, as determined by immunolocalization, or lacks expression of SOX2, as determined by RT-PCR; (c) express mRNA for ACTA2, ADAMTS1, AMOT, ANG, ANGPT1, ANGPT2, ANGPTL1, ANGPTL2, ANGPTL4, BAIL CD44, CD200, CEACAM1, CHGA, COL15A1, COL18A1, COL4A1, COL4A2, COL4A3, CSF3, CTGF, CXCL12, CXCL2, DNMT3B, ECGF1, EDG1, EDIL3, ENPP2, EPHB2, FBLN5, F2, FGF1, FGF2, FIGF, FLT4, FN1, FST, FOXC2, GRN, HGF, HEY1, HSPG2, IFNB1, IL8, IL12A, ITGA4, ITGAV, ITGB3, MDK, MMP2, MYOZ2, NRP1, NRP2, PDGFB, PDGFRA, PDGFRB, PECAM1, PF4, PGK1, PROX1, PTN, SEMA3F, SERPINB5, SERPINC1, SERPINF1, TIMP2, TIMP3, TGFA, TGFB1, THBS1, THBS2, TIE1, TIE2/TEK, TNF, TNNI1, TNFSF15, VASH1, VEGF, VEGFB, VEGFC, VEGFR1/FLT1, and/or VEGFR2/KDR; (d) expresses one or more of CD49d, Connexin-43, HLA-ABC, Beta 2-microglobulin, CD349, CD318, PDL1, CD106, Galectin-1, ADAM 17, angiotensinogen precursor, filamin A, alpha-actinin 1, megalin, macrophage acetylated LDL receptor I and II, activin receptor type IIB precursor, Wnt-9 protein, glial fibrillary acidic protein, astrocyte, myosin-binding protein C, and/or myosin heavy chain, nonmuscle type A; (e) secretes VEGF, HGF, IL-8, MCP-3, FGF2, Follistatin, G-CSF, EGF, ENA-78, GRO, IL-6, MCP-1, PDGF-BB, TIMP-2, uPAR, and/or Galectin-1, e.g., into culture medium in which the cell grows; (f) expresses micro RNAs miR-17-3p, miR-18a, miR-18b, miR-19b, miR-92, and/or miR-296 at a higher level than an equivalent number of bone marrow-derived mesenchymal stem cells; (g) expresses micro RNAs miR-20a, miR-20b, miR-221, miR-222, miR-15b, and/or miR-16 at a lower level than an equivalent number of bone marrow-derived mesenchymal stem cells; (h) expresses miRNAs miR-17-3p, miR-18a, miR-18b, miR-19b, miR-92, miR-20a, miR-20b, miR-296, miR-221, miR-222, miR-15b, and/or miR-16; and/or (i) expresses increased levels of CD202b, IL-8 and/or VEGF when cultured in less than about 5% O₂, compared to expression of CD202b, IL-8 and/or VEGF under 21% O₂. Further provided herein are populations of cells comprising AMDACs, e.g. populations of AMDACs, having one or more of the above-recited characteristics.

In another embodiment, any of the above populations of cells comprising amnion derived adherent cells secretes angiogenic factors. In specific embodiments, the population of cells secretes vascular endothelial growth factor (VEGF), epithelial growth factor (EGF), platelet derived growth factor (PDGF), basic fibroblast growth factor (bFGF), and/or interleukin-8 (IL-8). In other specific embodiments, the population of cells comprising amnion-derived angiogenic cells secretes one or more angiogenic factors and thereby induces human endothelial cells to migrate in an in vitro wound healing assay. In other specific embodiments, the population of cells comprising amnion derived adherent cells induces maturation, differentiation or proliferation of human endothelial cells, endothelial progenitors, myocytes or myoblasts.

In another embodiment, any of the above populations of cells comprising amnion derived adherent cells take up acetylated low density lipoprotein (LDL) when cultured in the presence of extracellular matrix proteins, e.g., collagen type I or IV, and/or one or more angiogenic factors, e.g., VEGF, EGF, PDGF, or bFGF, e.g., on a substrate such as placental collagen or MATRIGEL™.

In another embodiment, provided herein is a population of cells comprising amnion derived adherent cells, wherein said cells are adherent to tissue culture plastic, and wherein said cells are OCT-4⁻, as determined by RT-PCR, and VEGFR2/KDR⁺, CD9⁺, CD54⁺, CD105⁺, CD200⁺, or VE-cadherin⁻, as determined by immunolocalization. In specific embodiments, at least 10%, 20%, 30%, 40%, 50%, 60%, 70%, 80%, 90%, 95%, 98% or 99% of the cells in said population of cells are amnion derived cells that are OCT-4⁻, as determined by RT-PCR, and VEGFR2/KDR⁺, CD9⁺, CD54⁺, CD105⁺, CD200⁺, or VE-cadherin⁻, as determined by immunolocalization. In another specific embodiment, at least 10%, 20%, 30%, 40%, 50%, 60%, 70%, 80%, 90%, 95%, 98% or 99% of the cells in said population are amnion derived cells that are OCT-4⁻, as determined by RT-PCR, and VEGFR2/KDR⁺, CD9⁺, CD54⁺, CD105⁺, CD200⁺, and VE-cadherin⁻, as determined by immunolocalization. In another specific embodiment, said cells that are OCT-4⁻, as determined by RT-PCR, and VEGFR2/KDR⁺, CD9⁺, CD54⁺, CD 105⁺, CD200⁺, or VE-cadherin⁻, as determined by immunolocalization, do not express CD34, as detected by immunolocalization, after exposure to 1 to 100 ng/mL VEGF for 4 to 21 days. In another specific embodiment, said cells are also VE-cadherin⁻.

The populations of cells provided herein, comprising amnion derived adherent cells, are able to form sprouts or tube-like structures resembling vessels or vasculature. In one embodiment, the populations of cells comprising amnion derived adherent cells form sprouts or tube-like structures when cultured in the presence of an angiogenic moiety, e.g., VEGF, EGF, PDGF or bFGF. In a more specific embodiment, said amnion derived cells that are OCT-4⁻, as determined by RT-PCR, and VEGFR2/KDR⁺, CD9⁺, CD54⁺, CD105⁺, CD200⁺, or VE-cadherin⁻, as determined by immunolocalization, form sprouts or tube-like structures when said population of cells is cultured in the presence of vascular endothelial growth factor (VEGF).

The amnion derived adherent cells described herein display the above characteristics, e.g., combinations of cell surface markers and/or gene expression profiles, and/or angiogenic potency and function, in primary culture, or during proliferation in medium suitable for the culture of stem cells. Such medium includes, for example, medium comprising 1 to 100% DMEM-LG (Gibco), 1 to 100% MCDB-201 (Sigma), 1 to 10% fetal calf serum (FCS) (Hyclone Laboratories), 0.1 to 5× insulin-transferrin-selenium (ITS, Sigma), 0.1 to 5× linolenic-acid-bovine-serum-albumin (LA-BSA, Sigma), 10⁻⁵ to 10⁻¹⁵M dexamethasone (Sigma), 10⁻² to 10⁻¹⁰ M ascorbic acid 2-phosphate (Sigma), 1 to 50 ng/mL epidermal growth factor (EGF), (R&D Systems), 1 to 50 ng/mL platelet derived-growth factor (PDGF-BB) (R&D Systems), and 100 U penicillin/1000 U streptomycin. In a specific embodiment, the medium comprises 60% DMEM-LG (Gibco), 40% MCDB-201 (Sigma), 2% fetal calf serum (FCS) (Hyclone Laboratories), 1× insulin-transferrin-selenium (ITS), 1× linolenic-acid-bovine-serum-albumin (LA-BSA), 10⁻⁹M dexamethasone (Sigma), 10⁻⁴M ascorbic acid 2-phosphate (Sigma), epidermal growth factor (EGF) 10 ng/ml (R&D Systems), platelet derived-growth factor (PDGF-BB) 10 ng/ml (R&D Systems), and 100 U penicillin/1000 U streptomycin Other suitable media are described below.

The isolated populations of amnion derived adherent cells provided herein can comprise about, at least about, or no more than about, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more amnion derived adherent cells, e.g., in a container. In various embodiments, at least 10%, 20%, 30%, 40%, 50%, 60%, 70%, 80%, 90%, 95%, or 99% of the cells in the isolated cell populations provided herein are amnion derived adherent cells. That is, a population of isolated amnion derived adherent cells can comprise, e.g., as much as 1%, 5%, 10%, 20%, 30%, 40%, 50%, 60%, 70%, 80%, 90% non-stem cells.

The amnion derived adherent cells provided herein can be cultured on a substrate. In various embodiments, the substrate can be any surface on which culture and/or selection of amnion derived adherent cells, can be accomplished. Typically, the substrate is plastic, e.g., tissue culture dish or multiwell plate plastic. Tissue culture plastic can be treated, coated or imprinted with a biomolecule or synthetic mimetic agent, e.g., CELLSTART™, MESENCULT™ ACF-substrate, ornithine, or polylysine, or an extracellular matrix protein, e.g., collagen, laminin, fibronectin, vitronectin, or the like.

Amnion derived cells, e.g., the amnion derived adherent cells provided herein, and populations of such cells, can be isolated from one or more placentas. For example, an isolated population of the amnion derived cells provided herein can be a population of placental cells comprising such cells obtained from, or contained within, disrupted amnion tissue, e.g., tissue digestate (that is, the collection of cells obtained by enzymatic digestion of an amnion), wherein said population of cells is enriched for the amnion derived cells, and wherein the tissue is from a single placenta or from two or more placentas. Isolated amnion derived cells can be cultured and expanded to produce populations of such cells. Populations of placental cells comprising amnion derived adherent cells can also be cultured and expanded to produce populations of amnion derived adherent cells.

In certain embodiments, AMDACs displaying any of the above marker and/or gene expression characteristics have been passaged at least 1, 2, 3, 4, 5, 6, 7, 8, 9, 10, 11, 12, 13, 14, 15, 16, 17, 18, 19 or 20 times, or more. In certain other embodiments, AMDACs displaying any of the above marker and/or gene expression characteristics have been doubled in culture at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or at least 50 times, or more.

### 5.4 Methods of Obtaining Amnion-Derived Angiogenic Cells

The amnion derived adherent cells, and populations of cells comprising the amnion derived adherent cells, can be produced, e.g., isolated from other cells or cell populations, for example, through particular methods of digestion of amnion tissue, optionally followed by assessment of the resulting cells or cell population for the presence or absence of markers, or combinations of markers, characteristics of amnion derived adherent cells, or by obtaining amnion cells and selecting on the basis of markers characteristic of amnion derived adherent cells.

The amnion derived adherent cells, and isolated populations of cells comprising the amnion derived adherent cells, provided herein can be produced by, e.g., digestion of amnion tissue followed by selection for adherent cells. In one embodiment, for instance, isolated amnion derived adherent cells, or an isolated population of cells comprising amnion derived adherent cells, can be produced by (1) digesting amnion tissue with a first enzyme to dissociate cells from the epithelial layer of the amnion from cells from the mesenchymal layer of the amnion; (2) subsequently digesting the mesenchymal layer of the amnion with a second enzyme to form a single-cell suspension; (3) culturing cells in said single-cell suspension on a tissue culture surface, e.g., tissue culture plastic; and (4) selecting cells that adhere to said surface after a change of medium, thereby producing an isolated population of cells comprising amnion derived adherent cells. In a specific embodiment, said first enzyme is trypsin. In a more specific embodiment, said trypsin is used at a concentration of 0.25% trypsin (w/v), in 5-20, e.g., 10 milliliters solution per gram of amnion tissue to be digested. In another more specific embodiment, said digesting with trypsin is allowed to proceed for about 15 minutes at 37° C. and is repeated up to three times. In another specific embodiment, said second enzyme is collagenase. In a more specific embodiment, said collagenase is used at a concentration between 50 and 500 U/L in 5 mL per gram of amnion tissue to be digested. In another more specific embodiment, said digesting with collagenase is allowed to proceed for about 45-60 minutes at 37° C. In another specific embodiment, the single-cell suspension formed after digestion with collagenase is filtered through, e.g., a 75 μM-150 μM filter between step (2) and step (3). In another specific embodiment, said first enzyme is trypsin, and said second enzyme is collagenase.

An isolated population of cells comprising amnion derived adherent cells can, in another embodiment, be obtained by selecting cells from amnion, e.g., cells obtained by digesting amnion tissue as described elsewhere herein, that display one or more characteristics of an amnion derived adherent cell. In one embodiment, for example, a cell population is produced by a method comprising selecting amnion cells that are (a) negative for OCT-4, as determined by RT-PCR, and (b) positive for one or more of VEGFR2/KDR, CD9, CD54, CD105, CD200, as determined by immunolocalization; and isolating said cells from other cells to form a cell population. In a specific embodiment, said amnion cells are additionally VE-cadherin⁻. In a specific embodiment, a cell population is produced by selecting placental cells that are (a) negative for OCT-4, as determined by RT-PCR, and VE-cadherin, as determined by immunolocalization, and (b) positive for each of VEGFR2/KDR, CD9, CD54, CD105, CD200, as determined by immunolocalization; and isolating said cells from other cells to form a cell population. In certain embodiments, selection by immunolocalization is performed before selection by RT-PCR. In another specific embodiment, said selecting comprises selecting cells that do not express cellular marker CD34 after culture for 4 to 21 days in the presence of 1 to 100 ng/mL VEGF.

In another embodiment, for example, a cell population is produced by a method comprising selecting amnion cells that are adherent to tissue culture plastic and are OCT-4⁻, as determined by RT-PCR, and VEGFR1/Flt-1⁺ and VEGFR2/KDR⁺, as determined by immunolocalization, and isolating said cells from other cells to form a cell population. In a specific embodiment, a cell population is produced by a method comprising selecting amnion cells that are OCT-4⁻, as determined by RT-PCR, and VEGFR1/Flt-1⁺, VEGFR2/KDR⁺, and HLA-G⁻, as determined by immunolocalization. In another specific embodiment, said cell population is produced by selecting amnion cells that are additionally one or more, or all, of CD9⁺, CD10⁺, CD44⁺, CD54⁺, CD98⁺, Tie-2⁺, TEM-7⁺, CD31⁻, CD34⁻, CD45⁻, CD133⁻, CD 143⁻, CD 146⁻, and/or CXCR4⁻ (chemokine (C-X-C motif) receptor 4) as determined by immunolocalization, and isolating the cells from cells that do not display one or more of these characteristics. In another specific embodiment, said cell population is produced by selecting amnion cells that are additionally VE-cadherin⁻ as determined by immunolocalization, and isolating the cells from cells that are VE-cadherin⁺. In another specific embodiment, said cell population is produced by selecting amnion cells that are additionally CD105⁺ and CD200⁺ as determined by immunolocalization, and isolating the cells from cells that are CD105⁻ or CD200⁻. In another specific embodiment, said cell does not express CD34 as detected by immunolocalization after exposure to 1 to 100 ng/mL VEGF for 4 to 21 days.

In the selection of cells, it is not necessary to test an entire population of cells for characteristics specific to amnion derived adherent cells. Instead, one or more aliquots of cells (e.g., about 0.5%-2%) of a population of cells may be tested for such characteristics, and the results can be attributed to the remaining cells in the population.

Selected cells can be confirmed to be the amnion derived adherent cells provided herein by culturing a sample of the cells (e.g., about 10⁴ to about 10⁵ cells) on a substrate, e.g., MATRIGEL™, for 4 to 14, e.g., 7, days in the presence of VEGF (e.g., about 50 ng/mL), and visually inspecting the cells for the appearance of sprouts and/or cellular networks.

Amnion derived adherent cells can be selected by the above markers using any method known in the art of cell selection. For example, the adherent cells can be selected using an antibody or antibodies to one or more cell surface markers, for example, in immunolocalization, e.g., flow cytometry or FACS. Selection can be accomplished using antibodies in conjunction with magnetic beads. Antibodies that are specific for certain markers are known in the art and are available commercially, e.g., antibodies to CD9 (Abcam); CD54 (Abcam); CD105 (Abcam; BioDesign International, Saco, Me., etc.); CD200 (Abcam) cytokeratin (SigmaAldrich). Antibodies to other markers are also available commercially, e.g., CD34, CD38 and CD45 are available from, e.g., StemCell Technologies or BioDesign International. Primers to OCT-4 sequences suitable for RT-PCR can be obtained commercially, e.g., from Millipore or Invitrogen, or can be readily derived from the human sequence in GenBank Accession No. DQ486513.

Detailed methods of obtaining placenta and amnion tissue, and treating such tissue in order to obtain amnion derived adherent cells, are provided below.

5.4.1 Cell Collection Composition

Generally, cells can be obtained from amnion from a mammalian placenta, e.g., a human placenta, using a physiologically-acceptable solution, e.g., a cell collection composition. Preferably, the cell collection composition prevents or suppresses apoptosis, prevents or suppresses cell death, lysis, decomposition and the like. A cell collection composition is described in detail in related U.S. Patent Application Publication No. 2007/0190042, entitled “Improved Medium for Collecting Placental Stem Cells and Preserving Organs,” the disclosure of which is incorporated herein by reference in its entirety.

The cell collection composition can comprise any physiologically-acceptable solution suitable for the collection and/or culture of amnion derived adherent cells, for example, a saline solution (e.g., phosphate-buffered saline, Kreb's solution, modified Kreb's solution, Eagle's solution, 0.9% NaCl. etc.), a culture medium (e.g., DMEM, H.DMEM, etc.), and the like, with or without the addition of a buffering component, e.g., 4-(2-hydroxyethyl)-1-piperazineethanesulfonic acid (HEPES).

The cell collection composition can comprise one or more components that tend to preserve cells, e.g., amnion derived adherent cells, that is, prevent the cells from dying, or delay the death of the cells, reduce the number of cells in a population of cells that die, or the like, from the time of collection to the time of culturing. Such components can be, e.g., an apoptosis inhibitor (e.g., a caspase inhibitor or JNK inhibitor); a vasodilator (e.g., magnesium sulfate, an antihypertensive drug, atrial natriuretic peptide (ANP), adrenocorticotropin, corticotropin-releasing hormone, sodium nitroprusside, hydralazine, adenosine triphosphate, adenosine, indomethacin or magnesium sulfate, a phosphodiesterase inhibitor, etc.); a necrosis inhibitor (e.g., 2-(1H-Indol-3-yl)-3-pentylamino-maleimide, pyrrolidine dithiocarbamate, or clonazepam); a TNF-α inhibitor; and/or an oxygen-carrying perfluorocarbon (e.g., perfluorooctyl bromide, perfluorodecyl bromide, etc.).

The cell collection composition can comprise one or more tissue-degrading enzymes, e.g., a metalloprotease, a serine protease, a neutral protease, an RNase, or a DNase, or the like. Such enzymes include, but are not limited to, collagenases (e.g., collagenase I, II, III or IV, a collagenase from Clostridium histolyticum, etc.); dispase, thermolysin, elastase, trypsin, LIBERASE™, hyaluronidase, and the like. The use of cell collection compositions comprising tissue-digesting enzymes is discussed in more detail below.

The cell collection composition can comprise a bacteriocidally or bacteriostatically effective amount of an antibiotic. In certain non-limiting embodiments, the antibiotic is a macrolide (e.g., tobramycin), a cephalosporin (e.g., cephalexin, cephradine, cefuroxime, cefprozil, cefaclor, cefixime or cefadroxil), a clarithromycin, an erythromycin, a penicillin (e.g., penicillin V) or a quinolone (e.g., ofloxacin, ciprofloxacin or norfloxacin), a tetracycline, a streptomycin, etc. In a particular embodiment, the antibiotic is active against Gram(+) and/or Gram(−) bacteria, e.g., Pseudomonas aeruginosa, Staphylococcus aureus, and the like.

The cell collection composition can also comprise one or more of the following compounds: adenosine (about 1 mM to about 50 mM); D-glucose (about 20 mM to about 100 mM); magnesium ions (about 1 mM to about 50 mM); a macromolecule of molecular weight greater than 20,000 daltons, in one embodiment, present in an amount sufficient to maintain endothelial integrity and cellular viability (e.g., a synthetic or naturally occurring colloid, a polysaccharide such as dextran or a polyethylene glycol present at about 25 g/l to about 100 g/l, or about 40 g/l to about 60 g/l); an antioxidant (e.g., butylated hydroxyanisole, butylated hydroxytoluene, glutathione, vitamin C or vitamin E present at about 25 μM to about 100 μM); a reducing agent (e.g., N-acetylcysteine present at about 0.1 mM to about 5 mM); an agent that prevents calcium entry into cells (e.g., verapamil present at about 2 μM to about 25 μM); nitroglycerin (e.g., about 0.05 g/L to about 0.2 g/L); an anticoagulant, in one embodiment, present in an amount sufficient to help prevent clotting of residual blood (e.g., heparin or hirudin present at a concentration of about 1000 units/l to about 100,000 units/l); or an amiloride containing compound (e.g., amiloride, ethyl isopropyl amiloride, hexamethylene amiloride, dimethyl amiloride or isobutyl amiloride present at about 1.0 μM to about 5 μM).

The amnion derived adherent cells described herein can also be collected, e.g., during and after digestion as described below, into a simple physiologically-acceptable buffer, e.g., phosphate-buffered saline, a 0.9% NaCl solution, cell culture medium, or the like.

5.4.2 Collection and Handling of Placenta

Generally, a human placenta is recovered shortly after its expulsion after birth, or after, e.g., Caesarian section. In a preferred embodiment, the placenta is recovered from a patient after informed consent and after a complete medical history of the patient is obtained and is associated with the placenta. Preferably, the medical history continues after delivery. Such a medical history can be used to coordinate subsequent use of the placenta or cells harvested therefrom. For example, human placental cells, e.g., amnion derived adherent cells, can be used, in light of the medical history, for personalized medicine for the infant, or a close relative, associated with the placenta, or for parents, siblings, or other relatives of the infant.

Prior to recovery of amnion derived adherent cells, the umbilical cord blood and placental blood are removed. In certain embodiments, after delivery, the cord blood in the placenta is recovered. The placenta can be subjected to a conventional cord blood recovery process. Typically a needle or cannula is used, with the aid of gravity, to exsanguinate the placenta (see, e.g., Anderson, U.S. Pat. No. 5,372,581; Hessel et al., U.S. Pat. No. 5,415,665). The needle or cannula is usually placed in the umbilical vein and the placenta can be gently massaged to aid in draining cord blood from the placenta. Such cord blood recovery may be performed commercially, e.g., LifeBank USA, Cedar Knolls, N.J., ViaCord, Cord Blood Registry and Cryocell. Preferably, the placenta is gravity drained without further manipulation so as to minimize tissue disruption during cord blood recovery.

Typically, a placenta is transported from the delivery or birthing room to another location, e.g., a laboratory, for recovery of cord blood and collection of cells by, e.g., perfusion or tissue dissociation. The placenta is preferably transported in a sterile, thermally insulated transport device (maintaining the temperature of the placenta between 20-28° C.), for example, by placing the placenta, with clamped proximal umbilical cord, in a sterile zip-lock plastic bag, which is then placed in an insulated container. In another embodiment, the placenta is transported in a cord blood collection kit substantially as described in U.S. Pat. No. 7,147,626. Preferably, the placenta is delivered to the laboratory four to twenty-four hours following delivery. In certain embodiments, the proximal umbilical cord is clamped, preferably within 4-5 cm (centimeter) of the insertion into the placental disc prior to cord blood recovery. In other embodiments, the proximal umbilical cord is clamped after cord blood recovery but prior to further processing of the placenta.

The placenta, prior to cell collection, can be stored under sterile conditions and at a temperature of, e.g., 4 to 25° C. (centigrade), e.g., at room temperature. The placenta may be stored for, e.g., a period of for zero to twenty-four hours, up to forty-eight hours, or longer than forty eight hours, prior to perfusing the placenta to remove any residual cord blood. In one embodiment, the placenta is harvested from between about zero hours to about two hours post-expulsion. The placenta can be stored in an anticoagulant solution at a temperature of, e.g., 4 to 25° C. (centigrade). Suitable anticoagulant solutions are well known in the art. For example, a solution of sodium citrate, heparin or warfarin sodium can be used. In a preferred embodiment, the anticoagulant solution comprises a solution of heparin (e.g., 1% w/w in 1:1000 solution). The exsanguinated placenta is preferably stored for no more than 36 hours before cells are collected.

5.4.3 Physical Disruption and Enzymatic Digestion of Amnion Tissue

In one embodiment, the amnion is separated from the rest of the placenta, e.g., by blunt dissection, e.g., using the fingers. The amnion can be dissected, e.g., into parts or tissue segments, prior to enzymatic digestion and adherent cell recovery. Amnion derived adherent cells can be obtained from a whole amnion, or from a small segment of amnion, e.g., a segment of amnion that is about 1, 2, 3, 4, 5, 6, 7, 8, 9, 10, 20, 30, 40, 50, 60, 70, 80, 90, 100, 200, 300, 400, 500, 600, 700, 800, 900 or about 1000 square millimeters in area.

Amnion derived adherent cells can generally be collected from a placental amnion or a portion thereof, at any time within about the first three days post-expulsion, but preferably between about 0 hours and 48 hours after expulsion, or about 8 hours and about 18 hours post-expulsion.

In one embodiment, amnion derived adherent cells are extracted from amnion tissue by enzymatic digestion using one or more tissue-digesting enzymes. The amnion, or a portion thereof, may, e.g., be digested with one or more enzymes dissolved or mixed into a cell collection composition as described above.

In certain embodiments, the cell collection composition comprises one or more tissue-disruptive enzyme(s). Enzymatic digestion preferably uses a combination of enzymes, e.g., a combination of a matrix metalloprotease and a neutral protease, for example, a combination of dispase and collagenase, e.g., used in sequential order. When more than one protease is used, the proteases may be used at the same time to digest the amnion tissue, or may be used serially. In one embodiment, for example, the amnion tissue is digested three times with trypsin and then once with collagenase.

In one embodiment, amnion tissue is enzymatically digested with one or more of a matrix metalloprotease, a neutral protease, and a mucolytic enzyme. In a specific embodiment, the amnion tissue is digested with a combination of collagenase, dispase, and hyaluronidase. In another specific embodiment, the amnion tissue is digested with a combination of LIBERASE™ (Boehringer Mannheim Corp., Indianapolis, Ind.) and hyaluronidase. Other enzymes that can be used to disrupt amnion tissue include papain, deoxyribonucleases, serine proteases, such as trypsin, chymotrypsin, or elastase. Serine proteases may be inhibited by alpha 2 microglobulin in serum and therefore the medium used for digestion can, in certain embodiments, be serum-free. In certain other embodiments, EDTA and DNase are used in the digestion of amnion tissue, e.g., to increase the efficiency of cell recovery. In certain other embodiments, the digestate is diluted so as to avoid trapping cells within the viscous digest.

Typical concentrations for tissue digestion enzymes include, e.g., 50-200 U/mL for collagenase I and collagenase IV, 1-10 U/mL for dispase, and 10-100 U/mL for elastase. Proteases can be used in combination, that is, two or more proteases in the same digestion reaction, or can be used sequentially in order to isolate amnion derived adherent cells. For example, in one embodiment, amnion tissue, or part thereof, is digested first with an appropriate amount of trypsin, at a concentration of about 0.25%, for, e.g., 15 minutes, at 37° C., followed by collagenase I at about 1 to about 2 mg/ml for, e.g., 45 minutes.

In one embodiment, amnion derived adherent cells can be obtained as follows. The amniotic membrane is cut into segments approximately 0.1″×0.1″ to about 5″×5″, e.g., 2″×2″, in size. The epithelial monolayer is removed from the fetal side of the amniotic membrane by triple trypsinization as follows. The segments of amniotic membrane are placed into a container with warm (e.g., about 20° C. to about 37° C.) trypsin-EDTA solution (0.25%). The volume of trypsin can range from about 5 mL per gram of amnion to about 50 mL per gram of amnion. The container is agitated for about 5 minutes to about 30 minutes, e.g., 15 minutes, while maintaining the temperature constant. The segments of amniotic membrane are then separated from the trypsin solution by any appropriate method, such as manually removing the amnion segments, or by filtration. The trypsinization step can be repeated at least one more time.

Upon completion of the final trypsinization, the segments of amniotic membrane are placed back into the container filled with warm trypsin neutralization solution, such as phosphate-buffered saline (PBS)/10% FBS, PBS/5% FBS or PBS/3% FBS. The container is agitated for about 5 seconds to about 30 minutes, e.g., 5 minutes. The segments of amniotic membrane are then separated from the trypsin neutralization solution as described above, and the segments of amniotic membrane are placed into the container filled with warm PBS, pH 7.2. The container is agitated for about 5 seconds to about 30 minutes, and the amniotic membrane segments are then separated from the PBS as described above.

The segments of amniotic membrane are then placed into the container filled with warm (e.g., about 20° C. to about 37° C.) digestion solution. The volume of digestion solution can range from about 5 mL per gram of amnion to about 50 mL per gram of amnion. Digestion solutions comprise digestion enzymes in an appropriate culture medium, such as DMEM. Typical digestion solutions include collagenase type I (about 50 U/mL to about 500 U/mL); collagenase type I (about 50 U/mL to about 500 U/mL) plus dispase (about 5 U/mL to about 100 U/mL); and collagenase type I (about 50 U/mL to about 500 U/mL), dispase (about 2 U/mL to about 50 U/mL) and hyaluronidase (about 3 U/mL to about 10 U/mL). The container is agitated at 37° C. until amnion digestion is substantially complete (approximately 10 minutes to about 90 minutes). Warm PBS/5% FBS is then added to the container at a ratio of about 1 mL per gram of amniotic tissue to about 50 mL per gram of amniotic tissue. The container is agitated for about 2 minutes to about 5 minutes. The cell suspension is then filtered to remove any undigested tissue using a 40 μm to 100 μm filter. The cells are suspended in warm PBS (about 1 mL to about 500 mL), and then centrifuged at 200×g to about 400×g for about 5 minutes to about 30 minutes, e.g. 300×g for about 15 minutes at 20° C. After centrifugation, the supernatant is removed and the cells are resuspended in a suitable culture medium. The cell suspension can be filtered (40 μm to 70 μm filter) to remove any remaining undigested tissue, yielding a single cell suspension.

In this embodiment, cells in suspension are collected and cultured as described elsewhere herein to produce isolated amnion derived adherent cells, and populations of such cells. The remaining undigested amnion, in this embodiment, can be discarded. The cells released from the amnion tissue can be, e.g., collected, e.g., by centrifugation, and cultured in standard cell culture medium.

In any of the digestion protocols herein, the cell suspension obtained by digestion can be filtered, e.g., through a filter comprising pores from about 50 μm to about 150 μm, e.g., from about 75 μm to about 125 μm. In a more specific embodiment, the cell suspension can be filtered through two or more filters, e.g., a 125 μm filter and a 75 μm filter.

In conjunction with any of the methods described herein, AMDACs can be isolated from the cells released during digestion by selecting cells that express one or more characteristics of AMDACs, as described in Section 5.3, above.

AMDACs can also, for example, be isolated using a specific two-step isolation method comprising digestion with trypsin followed by digestion with collagenase. Thus, in another aspect, provided herein is a method of isolating amnion derived adherent cells comprising digesting an amniotic membrane or portion thereof with trypsin such that epithelial cells are released from said amniotic membrane; removing the amniotic membrane or portion thereof from said epithelial cells; further digesting the amniotic membrane or portion thereof with collagenase such that amnion derived adherent cells are released from said amniotic membrane or portion thereof; and separating said amnion derived adherent cells from said amniotic membrane. In a specific embodiment, digestion of the amniotic membrane or portion thereof is repeated at least once. In another specific embodiment, digestion of the amniotic membrane or portion thereof with collagenase is repeated at least once. In another specific embodiment, the trypsin is at about 0.1%-1.0% (final concentration). In a more specific embodiment, the trypsin is at about 0.25% (final concentration). In another specific embodiment, the collagenase is at about 50 U/mL to about 1000 U/mL (final concentration). In a more specific embodiment, the collagenase is at about 125 U/mL (final concentration). In another specific embodiment, the method of isolation additionally comprises culturing said amnion derived adherent cells in cell culture and separating said amnion derived adherent cells from non-adherent cells in said culture to produce an enriched population of amnion derived adherent cells. In more specific embodiments, at least 50%, 55%, 60%, 65%, 70%, 75%, 80%, 85%, 90%, 95%, 98% or 99% of cells in said enriched population of amnion derived adherent cells are said amnion derived adherent cells.

In a more specific embodiment of the above methods, the amnion derived adherent cells are negative for OCT-4, as determined by RT-PCR, and one or more of HLA-G⁺, CD90⁺, CD105⁺, and CD117⁻, as determined by flow cytometry.

5.4.4 Isolation, Sorting, and Characterization of Amnion Derived Adherent Cells

Cell pellets can be resuspended in fresh cell collection composition, as described above, or a medium suitable for cell maintenance, e.g., Dulbecco's Modified Eagle's Medium (DMEM); Iscove's Modified Dulbecco's Medium (IMDM), e.g. IMDM serum-free medium containing 2U/mL heparin and 2 mM EDTA (GibcoBRL, NY); a mixture of buffer (e.g. PBS, HBSS) with FBS (e.g. 2% v/v); or the like.

Amnion derived adherent cells that have been cultured, e.g., on a surface, e.g., on tissue culture plastic, with or without additional extracellular matrix coating such as fibronectin, can be passaged or isolated by differential adherence. For example, a cell suspension obtained from collagenase digestion of amnion tissue, performed as described in Section 5.4.3, above, can be cultured, e.g., for 3-7 days in culture medium on tissue culture plastic. During culture, a plurality of cells in the suspension adhere to the culture surface, and, after continued culture, give rise to amnion derived adherent cells. Nonadherent cells, which do not give rise to the amnion derived adherent cells, are removed during medium exchange.

The number and type of cells collected from amnion can be monitored, for example, by measuring changes in morphology and cell surface markers using standard cell detection techniques such as immunolocalization, e.g., flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, and/or by measuring changes in gene expression using techniques well known in the art, such as PCR and gene expression profiling. These techniques can be used, too, to identify cells that are positive for one or more particular markers. For example, using one or more antibodies to CD34, one can determine, using the techniques above, whether a cell comprises a detectable amount of CD34; if so, the cell is CD34⁺.

Amnion-derived cells, e.g., cells that have been isolated by Ficoll separation, differential adherence, or a combination of both, can be sorted using a fluorescence activated cell sorter (FACS). Fluorescence activated cell sorting (FACS) is a well-known method for separating particles, including cells, based on the fluorescent properties of the particles (see, e.g., Kamarch, 1987, Methods Enzymol, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In one sorting scheme, cells from placenta, e.g., amnion derived adherent cells, can be sorted on the basis of expression of the markers CD49f, VEGFR2/KDR, and/or Flt-1NEGFR1. Preferably the cells are identified as being OCT-4⁻, e.g., by determining the expression of OCT-4 by RT-PCR in a sample of the cells, wherein the cells are OCT-4⁻ if the cells in the sample fail to show detectable production of mRNA for OCT-4 after 30 cycles. For example, cells from amnion that are VEGFR2/KDR⁺ and VEGFR1/Flt-1⁺ can be sorted from cells that are one or more of VEGFR2/KDR⁻, and VEGFR1/Flt-1⁺, CD9⁺, CD54⁺, CD105⁺, CD200⁺, and/or VE-cadherin⁻. In a specific embodiment, amnion-derived, tissue culture plastic-adherent cells that are one or more of CD49f⁺, VEGFR2/KDR⁺, CD9⁺, CD54⁺, CD105⁺, CD200⁺, and/or VE-cadherin⁻, or cells that are VEGFR2/KDR⁺, CD9⁺, CD54⁺, CD105⁺, CD200⁺, and VE-cadherin⁻, are sorted away from cells not expressing one or more of such marker(s), and selected. In another specific embodiment, CD49f⁺, VEGFR2/KDR⁺, VEGFR1/Flt-1⁺ cells that are additionally one or more, or all, of CD31⁺, CD34⁺, CD45⁺, CD133⁻, and/or Tie-2⁺ are sorted from cells that do not display one or more, or any, of such characteristics. In another specific embodiment, VEGFR2/KDR⁺, VEGFR1/Flt-1⁺ cells that are additionally one or more, or all, of CD9⁺, CD10⁺, CD44⁺, CD54⁺, CD98⁺, Tie-2⁺, TEM-7⁺, CD31⁻, CD34⁻, CD45⁻, CD133⁻, CD 143⁻, CD 146⁻, and/or CXCR4⁻, are sorted from cells that do not display one or more, or any, of such characteristics.

Selection for amnion derived adherent cells can be performed on a cell suspension resulting from digestion, or on isolated cells collected from digestate, e.g., by centrifugation or separation using flow cytometry. Selection by expressed markers can be accomplished alone or, e.g., in connection with procedures to select cells on the basis of their adherence properties in culture. For example, an adherence selection can be accomplished before or after sorting on the basis of marker expression.

With respect to antibody-mediated detection and sorting of placental cells, any antibody, specific for a particular marker, can be used, in combination with any fluorophore or other label suitable for the detection and sorting of cells (e.g., fluorescence-activated cell sorting). Antibody/fluorophore combinations to specific markers include, but are not limited to, fluorescein isothiocyanate (FITC) conjugated monoclonal antibodies against CD105 (available from R&D Systems Inc., Minneapolis, Minn.); phycoerythrin (PE) conjugated monoclonal antibodies against CD200 (BD Biosciences Pharmingen); VEGFR2/KDR-Biotin (CD309, Abcam), and the like. Antibodies to any of the markers disclosed herein can be labeled with any standard label for antibodies that facilitates detection of the antibodies, including, e.g., horseradish peroxidase, alkaline phosphatase, β-galactosidase, acetylcholinesterase streptavidin/biotin, avidin/biotin, umbelliferone, fluorescein, fluorescein isothiocyanate (FITC), rhodamine, dichlorotriazinylamine fluorescein, dansyl chloride or phycoerythrin (PE), luminol, luciferase, luciferin, and aequorin, and examples of suitable radioactive material include ¹²⁵I, ¹³¹I, ³⁵S or ³H.

Amnion derived adherent cells can be labeled with an antibody to a single marker and detected and/sorted based on the single marker, or can be simultaneously labeled with multiple antibodies to a plurality of different markers and sorted based on the plurality of markers.

In another embodiment, magnetic beads can be used to separate cells, e.g., to separate the amnion derived adherent cells described herein from other amnion cells. The cells may be sorted using a magnetic activated cell sorting (MACS) technique, a method for separating particles based on their ability to bind magnetic beads (0.5-100 μm diameter). A variety of useful modifications can be performed on the magnetic microspheres, including covalent addition of antibody that specifically recognizes a particular cell surface molecule or hapten. The beads are then mixed with the cells to allow binding. Cells are then passed through a magnetic field to separate out cells having the specific cell surface marker. In one embodiment, these cells can then isolated and re-mixed with magnetic beads coupled to an antibody against additional cell surface markers. The cells are again passed through a magnetic field, isolating cells that bound both the antibodies. Such cells can then be diluted into separate dishes, such as microtiter dishes for clonal isolation.

Amnion derived adherent cells can be assessed for viability, proliferation potential, and longevity using standard techniques known in the art, such as trypan blue exclusion assay, fluorescein diacetate uptake assay, propidium iodide uptake assay (to assess viability); and thymidine uptake assay or MTT cell proliferation assay (to assess proliferation). Longevity may be determined by methods well known in the art, such as by determining the maximum number of population doubling in an extended culture.

Amnion derived adherent cells, can also be separated from other placental cells using other techniques known in the art, e.g., selective growth of desired cells (positive selection), selective destruction of unwanted cells (negative selection); separation based upon differential cell agglutinability in the mixed population as, for example, with soybean agglutinin; freeze-thaw procedures; filtration; conventional and zonal centrifugation; centrifugal elutriation (counter-streaming centrifugation); unit gravity separation; countercurrent distribution; electrophoresis; and the like.

### 5.5 Culture of Amnion Derived Adherent Cells

The growth of the amnion derived adherent cells described herein, as for any mammalian cell, depends in part upon the particular medium selected for growth. Under optimum conditions, amnion derived adherent cells typically double in number in approximately 24 hours. During culture, the amnion derived adherent cells described herein adhere to a substrate in culture, e.g. the surface of a tissue culture container (e.g., tissue culture dish plastic, fibronectin-coated plastic, and the like) and form a monolayer. Typically, the cells establish in culture within 2-7 days after digestion of the amnion. They proliferate at approximately 0.4 to 1.2 population doublings per day and can undergo at least 30 to 50 population doublings. The cells display a mesenchymal/fibroblastic cell-like phenotype during subconfluence and expansion, and a cuboidal/cobblestone-like appearance at confluence, and proliferation in culture is strongly contact-inhibited. Populations of amnion-derived angiogenic cells can form embryoid bodies during expansion in culture.

5.5.1 Culture Media

Isolated amnion derived adherent cells, or populations of such cells, can be used to initiate, or seed, cell cultures. Cells are generally transferred to sterile tissue culture vessels either uncoated or coated with extracellular matrix or biomolecules such as laminin, collagen (e.g., native or denatured), gelatin, fibronectin, ornithine, vitronectin, and extracellular membrane protein (e.g., MATRIGEL™ (BD Discovery Labware, Bedford, Mass.)).

AMDACs can, for example, be established in media suitable for the culture of stem cells, Establishment media can, for example, include EGM-2 medium (Lonza), DMEM+10% FBS, or medium comprising 60% DMEM-LG (Gibco), 40% MCDB-201 (Sigma), 2% fetal calf serum (FCS) (Hyclone Laboratories), 1× insulin-transferrin-selenium (ITS), 1× lenolenic-acid-bovine-serum-albumin (LA-BSA), 10⁻⁹ M dexamethasone (Sigma), 10⁻⁴M ascorbic acid 2-phosphate (Sigma), epidermal growth factor (EGF) 10 ng/ml (R&D Systems), platelet derived-growth factor (PDGF-BB) 10 ng/ml (R&D Systems), and 100 U penicillin/1000 U streptomycin (referred to herein as “standard medium”).

Amnion derived adherent cells can be cultured in any medium, and under any conditions, recognized in the art as acceptable for the culture of cells, e.g., adherent placental stem cells. Preferably, the culture medium comprises serum. In various embodiments, media for the culture or subculture of AMDACs includes STEMPRO® (Invitrogen), MSCM-sf (ScienCell, Carlsbad, Calif.), MESENCULT®-ACF medium (StemCell Technologies, Vancouver, Canada), standard medium, standard medium lacking EGF, standard medium lacking PDGF, DMEM+10% FBS, EGM-2 (Lonza), EGM-2MV (Lonza), 2%, 10% and 20% ES media, ES-SSR medium, or α-MEM-20% FBS. Medium acceptable for the culture of amnion derived adherent cells includes, e.g., DMEM, IMDM, DMEM (high or low glucose), Eagle's basal medium, Ham's F10 medium (F10), Ham's F-12 medium (F12), Iscove's modified Dulbecco's medium, Mesenchymal Stem Cell Growth Medium (MSCGM Lonza), ADVANCESTEM™ Medium (Hyclone), KNOCKOUT™ DMEM (Invitrogen), Leibovitz's L-15 medium, MCDB, DMEM/F12, RPMI 1640, advanced DMEM (Gibco), DMEM/MCDB201 (Sigma), and CELL-GRO FREE, or the like. In various embodiments, for example, DMEM-LG (Dulbecco's Modified Essential Medium, low glucose)/MCDB 201 (chick fibroblast basal medium) containing ITS (insulin-transferrin-selenium), LA+BSA (linoleic acid-bovine serum albumin), dextrose, L-ascorbic acid, PDGF, EGF, IGF-1, and penicillin/streptomycin; DMEM-HG (high glucose) comprising about 2 to about 20%, e.g., about 10%, fetal bovine serum (FBS; e.g. defined fetal bovine serum, Hyclone, Logan Utah); DMEM-HG comprising about 2 to about 20%, e.g., about 15%, FBS; IMDM (Iscove's modified Dulbecco's medium) comprising about 2 to about 20%, e.g., about 10%, FBS, about 2 to about 20%, e.g., about 10%, horse serum, and hydrocortisone; M199 comprising about 2 to about 20%, e.g., about 10%, FBS, EGF, and heparin; α-MEM (minimal essential medium) comprising about 2 to about 20%, e.g., about 10%, FBS, GLUTAMAX™ and gentamicin; DMEM comprising 10% FBS, GLUTAMAX™ and gentamicin; DMEM-LG comprising about 2 to about 20%, e.g., about 15%, (v/v) fetal bovine serum (e.g., defined fetal bovine serum, Hyclone, Logan Utah), antibiotics/antimycotics (e.g., penicillin at about 100 Units/milliliter, streptomycin at 100 micrograms/milliliter, and/or amphotericin B at 0.25 micrograms/milliliter (Invitrogen, Carlsbad, Calif.)), and 0.001% (v/v) β-mercaptoethanol (Sigma, St. Louis Mo.); KNOCKOUT™-DMEM basal medium supplemented with 2 to 20% FBS, non-essential amino acid (Invitrogen), beta-mercaptoethanol, KNOCKOUT™ basal medium supplemented with KNOCKOUT™ Serum Replacement, alpha-MEM comprising 2 to 20% FBS, EBM2™ basal medium supplemented with EGF, VEGF, bFGF, R3-IGF-1, hydrocortisone, heparin, ascorbic acid, FBS, gentamicin), or the like.

The culture medium can be supplemented with one or more components including, for example, serum (e.g., FCS or FBS, e.g., about 2-20% (v/v); equine (horse) serum (ES); human serum (HS)); beta-mercaptoethanol (BME), preferably about 0.001% (v/v); one or more growth factors, for example, platelet-derived growth factor (PDGF), epidermal growth factor (EGF), basic fibroblast growth factor (bFGF), insulin-like growth factor-1 (IGF-1), leukemia inhibitory factor (LIF), vascular endothelial growth factor (VEGF), and erythropoietin (EPO); amino acids, including L-valine; and one or more antibiotic and/or antimycotic agents to control microbial contamination, such as, for example, penicillin G, streptomycin sulfate, amphotericin B, gentamicin, and nystatin, either alone or in combination.

Amnion derived adherent cells (AMDACs) can be cultured in standard tissue culture conditions, e.g., in tissue culture dishes or multiwell plates. The cells can also be cultured using a hanging drop method. In this method, the cells are suspended at about 1×10⁴ cells per mL in about 5 ml, of medium, and one or more drops of the medium are placed on the inside of the lid of a tissue culture container, e.g., a 100 mL Petri dish. The drops can be, e.g., single drops, or multiple drops from, e.g., a multichannel pipetter. The lid is carefully inverted and placed on top of the bottom of the dish, which contains a volume of liquid, e.g., sterile PBS sufficient to maintain the moisture content in the dish atmosphere, and the cells are cultured. AMDACs can also be cultured in standard or high-volume or high-throughput culture systems, such as T-flasks, Corning HYPERFLASK®, Cell Factories (Nunc), 1-, 2-, 4-, 10 or 40-Tray Cell stacks, and the like. AMDACs may also be cultured in bioreactors, e.g., high-throughput bioreactors, static bioreactors, plug flow bioreactors, and the like. Examples of bioreactors include the Celligen Culture Systems (New Brunswick, Edison, N.J.), WAVE Bioreactor™ (General Electric), and the like.

In one embodiment, amnion derived adherent cells are cultured in the presence of a compound that acts to maintain an undifferentiated phenotype in the cells. In a specific embodiment, the compound is a substituted 3,4-dihydropyridimol[4,5-d]pyrimidine. In a more specific embodiment, the compound is a compound having the following chemical structure:

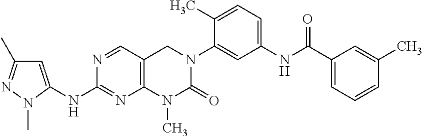

The compound can be contacted with an amnion derived adherent cell, or population of such cells, at a concentration of, for example, between about 1 μM to about 10 μM.

5.5.2 Expansion and Proliferation of Amnion Derived Adherent Cells

Once an isolated amnion derived adherent cell, or isolated population of such cells (e.g., amnion derived adherent cells, or population of such cells separated from at least 50% of the amnion cells with which the cell or population of cells is normally associated in vivo), the cells can be proliferated and expanded in vitro. For example, a population of adherent cells or amnion derived adherent cells can be cultured in tissue culture containers, e.g., dishes, flasks, multiwell plates, or the like, for a sufficient time for the cells to proliferate to 40-70% confluence, that is, until the cells and their progeny occupy 40-70% of the culturing surface area of the tissue culture container.

Amnion derived adherent cells can be seeded in culture vessels at a density that allows cell growth. For example, the cells may be seeded at low density (e.g., about 400 to about 6,000 cells/cm²) to high density (e.g., about 20,000 or more cells/cm²). In a preferred embodiment, the cells are cultured at about 0% to about 5% by volume CO₂ in air. In some preferred embodiments, the cells are cultured at about 0.1% to about 25% O₂ in air, preferably about 5% to about 20% O₂ in air. The cells are preferably cultured at about 25° C. to about 40° C., preferably at about 37° C.

The cells are preferably cultured in an incubator. During culture, the culture medium can be static or can be agitated, for example, during culture using a bioreactor. Amnion derived adherent cells preferably are grown under low oxidative stress (e.g., with addition of glutathione, ascorbic acid, catalase, tocopherol, N-acetylcysteine, or the like).

Although the amnion-derived angiogenic cells may be grown to confluence, the cells are preferably not grown to confluence. For example, once 40%-70% confluence is obtained, the cells may be passaged. For example, the cells can be enzymatically treated, e.g., trypsinized, using techniques well-known in the art, to separate them from the tissue culture surface. After removing the cells by pipetting and counting the cells, about 20,000-100,000 cells, preferably about 50,000 cells, or about 400 to about 6,000 cells/cm², can be passaged to a new culture container containing fresh culture medium. Typically, the new medium is the same type of medium from which the cells were removed. The amnion derived adherent cells can be passaged at least 1, 2, 3, 4, 5, 6, 7, 8, 9, 10, 11, 12, 13, 14, 15, 16, 17, 18, 19 or 20 times, or more. AMDACs can be doubled in culture at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or at least 50 times, or more.

### 5.6 Populations of Amnion Derived Adherent Cells Comprising Other Cell Types

The isolated cell populations comprising amnion derived adherent cells described herein can comprise a second cell type, e.g., placental cells that are not amnion derived adherent cells, or, e.g., cells that are not placental cells. For example, an isolated population of amnion derived adherent cells can comprise, e.g., can be combined with, a population of a second type of cells, wherein said second type of cell are, e.g., embryonic stem cells, blood cells (e.g., placental blood, placental blood cells, umbilical cord blood, umbilical cord blood cells, peripheral blood, peripheral blood cells, nucleated cells from placental blood, umbilical cord blood, or peripheral blood, and the like), stem cells isolated from blood (e.g., stem cells isolated from placental blood, umbilical cord blood or peripheral blood), placental stem cells (e.g., the placental stem cells described in U.S. Pat. No. 7,468,276, and in U.S. Patent Application Publication No. and 2007/0275362, the disclosures of which are incorporated herein by reference in their entireties), nucleated cells from placental perfusate, e.g., total nucleated cells from placental perfusate; umbilical cord stem cells, populations of blood-derived nucleated cells, bone marrow-derived mesenchymal stromal cells, bone marrow-derived mesenchymal stem cells, bone marrow-derived hematopoietic stem cells, crude bone marrow, adult (somatic) stem cells, populations of stem cells contained within tissue, cultured cells, e.g., cultured stem cells, populations of fully-differentiated cells (e.g., chondrocytes, fibroblasts, amniotic cells, osteoblasts, muscle cells, cardiac cells, etc.), pericytes, and the like. In a specific embodiment, a population of cells comprising amnion derived adherent cells comprises placental stem cells or stem cells from umbilical cord. In certain embodiments in which the second type of cell is blood or blood cells, erythrocytes have been removed from the population of cells.

In a specific embodiment, the second type of cell is a hematopoietic stem cell. Such hematopoietic stem cells can be, for example, contained within unprocessed placental, umbilical cord blood or peripheral blood; in total nucleated cells from placental blood, umbilical cord blood or peripheral blood; in an isolated population of CD34⁺ cells from placental blood, umbilical cord blood or peripheral blood; in unprocessed bone marrow; in total nucleated cells from bone marrow; in an isolated population of CD34⁺ cells from bone marrow, or the like.

In another embodiment, an isolated population of amnion derived adherent cells is combined with a plurality of adult or progenitor cells from the vascular system. In various embodiments, the cells are endothelial cells, endothelial progenitor cells, myocytes, cardiomyocytes, pericytes, angioblasts, myoblasts or cardiomyoblasts.

In a another embodiment, the second cell type is a non-embryonic cell type manipulated in culture in order to express markers of pluripotency and functions associated with embryonic stem cells

In specific embodiments of the above isolated populations of amnion derived adherent cells, either or both of the amnion derived adherent cells and cells of a second type are autologous, or are allogeneic, to an intended recipient of the cells.

Further provided herein is a composition comprising amnion derived adherent cells, and a plurality of stem cells other than the amnion derived adherent cells. In a specific embodiment, the composition comprises a stem cell that is obtained from a placenta, i.e., a placental stem cell, e.g., placental stem cells as described in U.S. Pat. Nos. 7,045,148; 7,255,879; and 7,311,905, and in U.S. Patent Application Publication No. 2007/0275362, the disclosures of each of which are incorporated herein by reference in their entireties. In specific embodiments, said placental stem cells are CD200⁺ and HLA-G; CD73⁺, CD105⁺, and CD200; CD200⁺ and OCT-4⁺; CD73⁺, CD105⁺ and HLA-G⁺; CD73⁺ and CD105⁺ and facilitate the formation of one or more embryoid-like bodies in a population of placental cells comprising said stem cell when said population is cultured under conditions that allow the formation of an embryoid-like body; or OCT-4⁺ and facilitate the formation of one or more embryoid-like bodies in a population of placental cells comprising the stem cell when said population is cultured under conditions that allow formation of embryoid-like bodies; or any combination thereof. In a more specific embodiment, said CD200⁺, HLA-G⁺ stem cells are CD34⁻, CD38⁻, CD45⁻, CD73⁺ and CD105⁺. In another more specific embodiment, said CD73⁺, CD105⁺, and CD200⁺ stem cells are CD34⁻, CD38⁻, CD45⁻, and HLA-G⁺. In another more specific embodiment, said CD200⁺, OCT-4⁺ stem cells are CD34⁻, CD38⁻, CD45⁻, CD73⁺, CD105⁺ and HLA-G⁺. In another more specific embodiment, said CD73⁺, CD105⁺ and HLA-G⁺ stem cells are CD34⁻, CD45⁻, OCT-4⁺ and CD200⁺. In another more specific embodiment, said CD73⁺ and CD105⁺ stem cells are OCT-4⁺, CD34⁻, CD38⁻ and CD45⁻. In another more specific embodiment, said OCT-4⁺ stem cells are CD73⁺, CD105⁺, CD200⁺, CD34⁻, CD38⁻, and CD45⁻. In another more specific embodiment, the placental stem cells are maternal in origin (that is, have the maternal genotype). In another more specific embodiment, the placental stem cells are fetal in origin (that is, have the fetal genotype).

In another specific embodiment, the composition comprises amnion derived adherent cells, and embryonic stem cells. In another specific embodiment, the composition comprises amnion derived adherent cells and mesenchymal stromal or stem cells, e.g., bone marrow-derived mesenchymal stromal or stem cells. In another specific embodiment, the composition comprises bone marrow-derived hematopoietic stem cells. In another specific embodiment, the composition comprises amnion derived adherent cells and hematopoietic progenitor cells, e.g., hematopoietic progenitor cells from bone marrow, fetal blood, umbilical cord blood, placental blood, and/or peripheral blood. In another specific embodiment, the composition comprises amnion derived adherent cells and somatic stem cells. In a more specific embodiment, said somatic stem cell is a neural stem cell, a hepatic stem cell, a pancreatic stem cell, an endothelial stem cell, a cardiac stem cell, or a muscle stem cell.

In other specific embodiments, the second type of cells comprise about, at least, or no more than, 10%, 15%, 20%, 25%, 30%, 35%, 40%, 45%, or 50% of cells in said population. In other specific embodiments, the AMDACs in said composition comprise at least 50%, 55%, 60%, 65%, 70%, 75%, 80%, 85% or 90% of cells in said composition. In other specific embodiments, the amnion derived adherent cells comprise about, at least, or no more than, 10%, 15%, 20%, 25%, 30%, 35%, 40%, or 45% of cells in said population.

Cells in an isolated population of amnion derived adherent cells can be combined with a plurality of cells of another type, e.g., with a population of stem cells, in a ratio of about 100,000,000:1, 50,000,000:1, 20,000,000:1, 10,000,000:1, 5,000,000:1, 2,000,000:1, 1,000,000:1, 500,000:1, 200,000:1, 100,000:1, 50,000:1, 20,000:1, 10,000:1, 5,000:1, 2,000:1, 1,000:1, 500:1, 200:1, 100:1, 50:1, 20:1, 10:1, 5:1, 2:1, 1:1; 1:2; 1:5; 1:10; 1:100; 1:200; 1:500; 1:1,000; 1:2,000; 1:5,000; 1:10,000; 1:20,000; 1:50,000; 1:100,000; 1:500,000; 1:1,000,000; 1:2,000,000; 1:5,000,000; 1:10,000,000; 1:20,000,000; 1:50,000,000; or about 1:100,000,000, comparing numbers of total nucleated cells in each population. Cells in an isolated population of amnion derived adherent cells can be combined with a plurality of cells of a plurality of cell types, as well.

### 5.7 Preservation of Amnion Derived Adherent Cells

Amnion derived adherent cells can be preserved, that is, placed under conditions that allow for long-term storage, or conditions that inhibit cell death by, e.g., apoptosis or necrosis, e.g., during collection or prior to production of the compositions described herein, e.g., using the methods described herein.

Amnion derived adherent cells can be preserved using, e.g., a composition comprising an apoptosis inhibitor, necrosis inhibitor and/or an oxygen-carrying perfluorocarbon, as described in U.S. Application Publication No. 2007/0190042, the disclosure of which is hereby incorporated by reference in its entirety. In one embodiment, a method of preserving such cells, or a population of such cells, comprises contacting said cells or population of cells with a cell collection composition comprising an inhibitor of apoptosis and an oxygen-carrying perfluorocarbon, wherein said inhibitor of apoptosis is present in an amount and for a time sufficient to reduce or prevent apoptosis in the population of cells, as compared to a population of cells not contacted with the inhibitor of apoptosis. In a specific embodiment, said inhibitor of apoptosis is a caspase inhibitor. In another specific embodiment, said inhibitor of apoptosis is a JNK inhibitor. In a more specific embodiment, said JNK inhibitor does not modulate differentiation or proliferation of amnion derived adherent cells. In another embodiment, said cell collection composition comprises said inhibitor of apoptosis and said oxygen-carrying perfluorocarbon in separate phases. In another embodiment, said cell collection composition comprises said inhibitor of apoptosis and said oxygen-carrying perfluorocarbon in an emulsion. In another embodiment, the cell collection composition additionally comprises an emulsifier, e.g., lecithin. In another embodiment, said apoptosis inhibitor and said perfluorocarbon are between about 0° C. and about 25° C. at the time of contacting the cells. In another more specific embodiment, said apoptosis inhibitor and said perfluorocarbon are between about 2° C. and 10° C., or between about 2° C. and about 5° C., at the time of contacting the cells. In another more specific embodiment, said contacting is performed during transport of said population of cells. In another more specific embodiment, said contacting is performed during freezing and thawing of said population of cells.

Populations of amnion derived adherent cells can be preserved, e.g., by a method comprising contacting a population of said cells with an inhibitor of apoptosis and an organ-preserving compound, wherein said inhibitor of apoptosis is present in an amount and for a time sufficient to reduce or prevent apoptosis in the population of cells, as compared to a population of cells not contacted with the inhibitor of apoptosis. In a specific embodiment, the organ-preserving compound is UW solution (described in U.S. Pat. No. 4,798,824; also known as ViaSpan; see also Southard et al., Transplantation 49(2):251-257 (1990)) or a solution described in Stern et al., U.S. Pat. No. 5,552,267. In another embodiment, said organ-preserving compound is hydroxyethyl starch, lactobionic acid, raffinose, or a combination thereof. In another embodiment, the cell collection composition additionally comprises an oxygen-carrying perfluorocarbon, either in two phases or as an emulsion.

In another embodiment of the method, amnion derived adherent cells are contacted with a cell collection composition comprising an apoptosis inhibitor and oxygen-carrying perfluorocarbon, organ-preserving compound, or combination thereof, during perfusion. In another embodiment, the amnion derived adherent cells are contacted with such a cell collection composition during a process of tissue disruption, e.g., enzymatic digestion of amnion tissue. In another embodiment, amnion derived adherent cells are contacted with said cell collection compound after collection by tissue disruption, e.g., enzymatic digestion of amnion tissue.

Typically, during collection of amnion derived adherent cells, enrichment and isolation, it is preferable to minimize or eliminate cell stress due to hypoxia and mechanical stress. In another embodiment of the method, therefore, an amnion derived adherent cell, or population of cells comprising the amnion derived adherent cells, is exposed to a hypoxic condition during collection, enrichment or isolation for less than six hours during said preservation, wherein a hypoxic condition is a concentration of oxygen that is, e.g., less than normal atmospheric oxygen concentration; less than normal blood oxygen concentration; or the like. In a more specific embodiment, said cells or population of said cells is exposed to said hypoxic condition for less than two hours during said preservation. In another more specific embodiment, said cells or population of said cells is exposed to said hypoxic condition for less than one hour, or less than thirty minutes, or is not exposed to a hypoxic condition, during collection, enrichment or isolation. In another specific embodiment, said population of cells is not exposed to shear stress during collection, enrichment or isolation.

Amnion derived adherent cells can be cryopreserved, in general or by the specific methods disclosed herein, e.g., in cryopreservation medium in small containers, e.g., ampoules. Suitable cryopreservation medium includes, but is not limited to, culture medium including, e.g., growth medium, or cell freezing medium, for example commercially available cell freezing medium, e.g., cell freezing medium identified by SigmaAldrich catalog numbers C2695, C2639 (Cell Freezing Medium-Serum-free 1×, not containing DMSO) or C6039 (Cell Freezing Medium-Glycoerol 1× containing Minimum Essential Medium, glycerol, calf serum and bovine serum), Lonza PROFREEZE™ 2× Medium, methylcellulose, dextran, human serum albumin, fetal bovine serum, fetal calf serum, or Plasmalyte. Cryopreservation medium preferably comprises DMSO (dimethylsulfoxide) or glycerol, at a concentration of, e.g., about 1% to about 20%, e.g., about 5% to 10% (v/v), optionally including fetal bovine serum or human serum. Cryopreservation medium may comprise additional agents, for example, methylcellulose and/or glycerol. Isolated amnion derived adherent cells are preferably cooled at about 1° C./min during cryopreservation. A preferred cryopreservation temperature is about −80° C. to about −180° C., preferably about −125° C. to about −140° C. Cryopreserved cells can be transferred to vapor phase of liquid nitrogen prior to thawing for use. In some embodiments, for example, once the ampoules have reached about −80° C., they are transferred to a liquid nitrogen storage area. Cryopreservation can also be done using a controlled-rate freezer. Cryopreserved cells preferably are thawed at a temperature of about 25° C. to about 40° C., preferably to a temperature of about 37° C.

### 5.8 Production of a Bank of Amnion Derived Adherent Cells

Amnion derived adherent cells can be cultured in a number of different ways to produce a set of lots, e.g., a set of individually-administrable doses, of such cells. Sets of lots of angiogenic amniotic cells, obtained from a plurality of placentas, can be arranged in a bank of cells for, e.g., long-term storage. Generally, amnion derived adherent cells are obtained from an initial culture of cells to form a seed culture, which is expanded under controlled conditions to form populations of cells from approximately equivalent numbers of doublings. Lots are preferably derived from the tissue of a single placenta, but can be derived from the tissue of a plurality of placentas.

In one non-limiting embodiment, lots or doses of amnion derived adherent cells are obtained as follows. Amnion tissue is first disrupted, e.g., digested as described in Section 5.4.3, above using serial trypsin and collagenase digestions. Cells from the collagenase-digested tissue are cultured, e.g., for about 1-3 weeks, preferably about 2 weeks. After removal of non-adherent cells, high-density colonies that form are collected, e.g., by trypsinization. These cells are collected and resuspended in a convenient volume of culture medium, and defined as Passage 0 cells.

Passage 0 cells can then be used to seed expansion cultures. Expansion cultures can be any arrangement of separate cell culture apparatuses, e.g., a Cell Factory by NUNC™. Cells in the Passage 0 culture can be subdivided to any degree so as to seed expansion cultures with, e.g., 1×10³, 2×10³, 3×10³, 4×10³, 5×10³, 6×10³, 7×10³, 8×10³, 9×10³, 1×10⁴, 1×10⁴, 2×10⁴, 3×10⁴, 4×10⁴, 5×10⁴, 6×10⁴, 7×10⁴, 8×10⁴, 9×10⁴, or 10×10⁴ adherent cells. Preferably, from about 1×10³ to about 3×10⁴ Passage 0 cells are used to seed each expansion culture. The number of expansion cultures can depend upon the number of Passage 0 cells, and may be greater or fewer in number depending upon the particular placenta(s) from which the adherent cells are obtained.

Expansion cultures can then be grown until the density of cells in culture reaches a certain value, e.g., about 1×10⁵ cells/cm². Cells can either be collected and cryopreserved at this point, or passaged into new expansion cultures as described above. Cells can be passaged, e.g., 2, 3, 4, 5, 6, 7, 8, 9, 10, 11, 12, 13, 14, 15, 16, 17, 18, 19 or 20 times prior to use. A record of the cumulative number of population doublings is preferably maintained during expansion culture(s). The cells from a Passage 0 culture can be expanded for 2, 3, 4, 5, 6, 7, 8, 9, 10, 12, 14, 16, 18, 20, 22, 24, 26, 28, 30, 32, 34, 36, 38 or 40 doublings, or up to 60 doublings. Preferably, however, the number of population doublings, prior to dividing the population of cells into individual doses, is between about 15 and about 30 doublings. The cells can be culture continuously throughout the expansion process, or can be frozen at one or more points during expansion.

Cells to be used for individual doses can be frozen, e.g., cryopreserved for later use. Individual doses can comprise, e.g., about 1 million to about 50 million cells per mL, and can comprise between about 10⁶ and about 10¹⁰ cells in total.

In one embodiment, therefore, a cell bank comprising amnion derived adherent cells can be made by a method comprising: expanding primary culture amnion derived adherent cells from a human post-partum placenta for a first plurality of population doublings; cryopreserving the cells to form a Master Cell Bank; optionally expanding a plurality of the cells from the Master Cell Bank for a second plurality of population doublings; cryopreserving the expanded cells to form a Working Cell Bank; optionally expanding a plurality of the expanded amnion derived adherent cells from the Working Cell Bank for a third plurality of population doublings; and cryopreserving the resulting expanded cells in individual doses, wherein said individual doses collectively compose a cell bank. The bank can comprise doses, or lots, of solely amnion derived adherent cells, or can comprise a combination of lots of amnion derived adherent cells and lots or doses of another kind of cell, e.g., another kind of stem or progenitor cell. Preferably, each individual dose comprises only amnion derived adherent cells. In another specific embodiment, all of said cells in said primary culture are from the same placenta. In another specific embodiment, said individual doses comprise from about 10⁴ to about 10⁵ cells. In another specific embodiment, said individual doses comprise from about 10⁵ to about 10⁶ cells. In another specific embodiment, said individual doses comprise from about 10⁶ to about 10⁷ cells. In another specific embodiment, said individual doses comprise from about 10⁷ to about 10⁸ cells. In another specific embodiment, said individual doses comprise from about 10⁸ to about 10⁹ cells. In another specific embodiment, said individual doses comprise from about 10⁹ to about 10¹⁰ cells.

In certain embodiments, amnion derived adherent cells can be thawed from a Working Cell Bank and cultured for a plurality of population doublings. When a desired number of cells is generated, or a desired number of population doublings has taken place, the adherent cells can be collected, e.g., by centrifugation, and resuspended in a solution comprising, e.g., dextran, e.g., 5% dextran. In certain embodiments, the dextran is dextran-40. In certain embodiments, the cells are collected a second time and resuspended in a solution comprising dextran and a cryopreservant, e.g., a 5% dextran (e.g., dextran-40) solution comprising 10% HSA and 2%-20%, e.g., 5% DMSO, and cryopreserved. The cryopreserved amnion derived adherent cells can be thawed, e.g., immediately before use.

In a preferred embodiment, the donor from which the placenta is obtained (e.g., the mother) is tested for at least one pathogen. In certain embodiments, if the mother tests positive for a tested pathogen, the entire lot from the placenta is discarded. Such testing can be performed at any time during production of lots of amnion derived adherent cells, including before or after establishment of Passage 0 cells, or during expansion culture. Pathogens for which the presence is tested can include, without limitation, hepatitis A, hepatitis B, hepatitis C, hepatitis D, hepatitis E, human immunodeficiency virus (types I and II), cytomegalovirus, herpesvirus, and the like.

### 5.9 Compositions Comprising Amnion Derived Adherent Cells

The method of treating individuals who have been exposed to radiation and/or the methods of hematopoietic reconstitution described herein encompass the use of compositions comprising AMDACs, e.g., liquids, solids (e.g., matrices), or a combination of both (e.g., hydrogels). In certain embodiments, AMDACs are contained within, or are components of, a pharmaceutical composition. Generally, compositions suitable for systemic administration are preferred for situations in which the individual's exposure to radiation was body-wide. However, pharmaceutical compositions comprising AMDACs, suitable for local administration, e.g., intramuscular, intraperitoneal, intradermal, subdermal administration, or the like.

The cells can be prepared in a form that is easily administrable to an individual, e.g., AMDACs in solution suitable for, e.g., intravenous administration, that are contained within a container that is suitable for medical use. Such a container can be, for example, a syringe, sterile plastic bag, flask, jar, or other container from which the AMDACs can be easily dispensed. For example, the container can be a blood bag or other plastic, medically-acceptable bag suitable for the intravenous administration of a liquid to a recipient. The container, in certain embodiments, is one that allows for cryopreservation of the cells. The cells in the compositions, e.g., pharmaceutical compositions, provided herein, can comprise amnion derived adherent cells derived from a single donor, or from multiple donors. The cells can be completely HLA-matched to an intended recipient, or partially or completely HLA-mismatched, e.g., can be completely autologous, partially allogeneic, or completely allogeneic.

Thus, in one embodiment, AMDACs in the compositions provided herein are administered to an individual in need thereof in the form of a composition comprising AMDACs in a container. In another specific embodiment, the container is a bag, flask, or jar. In more specific embodiment, said bag is a sterile plastic bag. In a more specific embodiment, said bag is suitable for, allows or facilitates intravenous administration of said AMDACs, e.g., by intravenous infusion, bolus injection, or the like. The bag can comprise multiple lumens or compartments that are interconnected to allow mixing of the cells and one or more other solutions, e.g., a drug, prior to, or during, administration. In another specific embodiment, prior to cryopreservation, the solution comprising the AMDACs comprises one or more compounds that facilitate cryopreservation of the cells. In another specific embodiment, said AMDACs are contained within a physiologically-acceptable aqueous solution. In a more specific embodiment, said physiologically-acceptable aqueous solution is a 0.9% NaCl solution. In another specific embodiment, said AMDACs are, or comprise cells that are, HLA-matched to a recipient of said cells. In another specific embodiment, said AMDACs are, or comprise cells that are, at least partially HLA-mismatched to a recipient of said cells. In another specific embodiment, said AMDACs are derived from a plurality of donors. In various specific embodiments, said container comprises about, at least, or at most 1×10⁶ said cells, 5×10⁶ said cells, 1×10⁷ said stem cells, 5×10⁷ said cells, 1×10⁸ said cells, 5×10⁸ said cells, 1×10⁹ said cells, 5×10⁹ said cells, or 1×10¹⁰ said cells. In other specific embodiments of any of the foregoing cryopreserved populations, said cells have been passaged about, at least, or no more than 5 times, no more than 10 times, no more than 15 times, or no more than 20 times. In another specific embodiment of any of the foregoing cryopreserved cells, said cells have been expanded within said container. In specific embodiments, a single unit dose of AMDACs can comprise, in various embodiments, about, at least, or no more than 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more AMDACs.

In certain embodiments, the pharmaceutical compositions provided herein comprises populations of AMDACs, that comprise 50% viable cells or more (that is, at least 50% of the cells in the population are functional or living). Preferably, at least 60% of the cells in the population are viable. More preferably, at least 70%, 80%, 90%, 95%, or 99% of the cells in the population in the pharmaceutical composition are viable.

5.9.1 Matrices Comprising Amnion Derived Adherent Cells

Further provided herein are compositions comprising matrices, hydrogels, scaffolds, and the like. Such compositions can be used in the place of, or in addition to, such cells in liquid suspension.

The matrix can be, e.g., a permanent or degradable decellularized tissue, e.g., a decellularized amniotic membrane, or a synthetic matrix. The matrix can be a three-dimensional scaffold. In a more specific embodiment, said matrix comprises collagen, gelatin, laminin, fibronectin, pectin, ornithine, or vitronectin. In another more specific embodiment, the matrix is an amniotic membrane or an amniotic membrane-derived biomaterial. In another more specific embodiment, said matrix comprises an extracellular membrane protein. In another more specific embodiment, said matrix comprises a synthetic compound. In another more specific embodiment, said matrix comprises a bioactive compound. In another more specific embodiment, said bioactive compound is a growth factor, a cytokine, an antibody, or an organic molecule of less than 5,000 daltons.

The amnion derived adherent cells described herein can be seeded onto a natural matrix, e.g., a placental biomaterial such as an amniotic membrane material. Such an amniotic membrane material can be, e.g., amniotic membrane dissected directly from a mammalian placenta; fixed or heat-treated amniotic membrane, substantially dry (i.e., <20% H₂O) amniotic membrane, chorionic membrane, substantially dry chorionic membrane, substantially dry amniotic and chorionic membrane, and the like. Preferred placental biomaterials on which the amnion derived adherent cells provided herein can be seeded are described in Hariri, U.S. Application Publication No. 2004/0048796, the disclosure of which is incorporated by reference herein in its entirety.

In another specific embodiment, the matrix is a composition comprising an extracellular matrix. In a more specific embodiment, said composition is MATRIGEL™ (BD Biosciences).

The isolated amnion derived adherent cells described herein can be suspended in a hydrogel solution suitable for, e.g., injection. The hydrogel is, e.g., an organic polymer (natural or synthetic) that is cross-linked via covalent, ionic, or hydrogen bonds to create a three-dimensional open-lattice structure that entraps water molecules to form a gel. Suitable hydrogels for such compositions include self-assembling peptides, such as RAD 16. In one embodiment, a hydrogel solution comprising the cells can be allowed to harden, for instance in a mold, to form a matrix having cells dispersed therein for implantation. The amnion derived adherent cells in such a matrix can also be cultured so that the cells are mitotically expanded, e.g., prior to implantation. Hydrogel-forming materials include polysaccharides such as alginate and salts thereof, peptides, polyphosphazines, and polyacrylates, which are crosslinked ionically, or block polymers such as polyethylene oxide-polypropylene glycol block copolymers which are crosslinked by temperature or pH, respectively. In some embodiments, the hydrogel or matrix is biodegradable.

In certain embodiments, the compositions comprising cells, provided herein, comprise an in situ polymerizable gel (see., e.g., U.S. Patent Application Publication 2002/0022676; Anseth et al., J. Control Release, 78(1-3):199-209 (2002); Wang et al., Biomaterials, 24(22):3969-80 (2003). In some embodiments, the polymers are at least partially soluble in aqueous solutions, such as water, buffered salt solutions, or aqueous alcohol solutions, that have charged side groups, or a monovalent ionic salt thereof. Examples of polymers having acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups.

In a specific embodiment, the matrix is a felt, which can be composed of a multifilament yarn made from a bioabsorbable material, e.g., PGA, PLA, PCL copolymers or blends, or hyaluronic acid. The yarn is made into a felt using standard textile processing techniques consisting of crimping, cutting, carding and needling. In another preferred embodiment the cells of the invention are seeded onto foam scaffolds that may be composite structures. In addition, the three-dimensional framework may be molded into a useful shape, such as a specific structure in the body to be repaired, replaced, or augmented. Other examples of scaffolds that can be used include nonwoven mats, porous foams, or self assembling peptides. Nonwoven mats can be formed using fibers comprised of a synthetic absorbable copolymer of glycolic and lactic acids (e.g., PGA/PLA) (VICRYL, Ethicon, Inc., Somerville, N.J.). Foams, composed of, e.g., poly(ε-caprolactone)/poly(glycolic acid) (PCL/PGA) copolymer, formed by processes such as freeze-drying, or lyophilization (see, e.g., U.S. Pat. No. 6,355,699), can also be used as scaffolds.

The amnion derived adherent cells described herein can be seeded onto a three-dimensional framework or scaffold and implanted in vivo. Such a framework can be implanted in combination with any one or more growth factors, cells, drugs or other components that, e.g., stimulate tissue formation, e.g., bone formation or formation of vasculature.

The placental amnion derived adherent cells provided herein can, in another embodiment, be seeded onto foam scaffolds that may be composite structures. Such foam scaffolds can be molded into a useful shape, such as that of a portion of a specific structure in the body to be repaired, replaced or augmented. In some embodiments, the framework is treated, e.g., with 0.1M acetic acid followed by incubation in polylysine, PBS, and/or collagen, prior to inoculation of the cells in order to enhance cell attachment. External surfaces of a matrix may be modified to improve the attachment or growth of cells and differentiation of tissue, such as by plasma-coating the matrix, or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate, etc.), a cellular matrix, and/or other materials such as, but not limited to, gelatin, alginates, agar, agarose, and plant gums, and the like.

In some embodiments, the matrix comprises, or is treated with, materials that render it non-thrombogenic. These treatments and materials may also promote and sustain endothelial growth, migration, and extracellular matrix deposition. Examples of these materials and treatments include but are not limited to natural materials such as basement membrane proteins such as laminin and Type IV collagen, synthetic materials such as EPTFE, and segmented polyurethaneurea silicones, such as PURSPAN™ (The Polymer Technology Group, Inc., Berkeley, Calif.). The matrix can also comprise anti-thrombotic agents such as heparin; the scaffolds can also be treated to alter the surface charge (e.g., coating with plasma) prior to seeding with the adherent cells provided herein.

The framework may be treated prior to inoculation of the amnion derived adherent cells provided herein in order to enhance cell attachment. For example, prior to inoculation with the cells of the invention, nylon matrices could be treated with 0.1 molar acetic acid and incubated in polylysine, PBS, and/or collagen to coat the nylon. Polystyrene can be similarly treated using sulfuric acid.

In addition, the external surfaces of the three-dimensional framework may be modified to improve the attachment or growth of cells and differentiation of tissue, such as by plasma coating the framework or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate), a cellular matrix, and/or other materials such as, but not limited to, gelatin, alginates, agar, agarose, or plant gums.

In some embodiments, the matrix comprises or is treated with materials that render the matrix non-thrombogenic, e.g., natural materials such as basement membrane proteins such as laminin and Type IV collagen, and synthetic materials such as ePTFE or segmented polyurethaneurea silicones, such as PURSPAN (The Polymer Technology Group, Inc., Berkeley, Calif.). Such materials can be further treated to render the scaffold non-thrombogenic, e.g., with heparin, and treatments that alter the surface charge of the material, such as plasma coating.

The therapeutic cell compositions comprising amnion derived adherent cells can also be provided in the form of a matrix-cell complex. Matrices can include biocompatible scaffolds, lattices, self-assembling structures and the like, whether bioabsorbable or not, liquid, gel, or solid. Such matrices are known in the arts of therapeutic cell treatment, surgical repair, tissue engineering, and wound healing. In certain embodiments, the cells adhere to the matrix. In other embodiments, the cells are entrapped or contained within matrix spaces. Most preferred are those matrix-cell complexes in which the cells grow in close association with the matrix and when used therapeutically, stimulate and support ingrowth of a recipient's cells, or stimulate or support angiogenesis. The matrix-cell compositions can be introduced into an individual's body in any way known in the art, including but not limited to implantation, injection, surgical attachment, transplantation with other tissue, injection, and the like. In some embodiments, the matrices form in vivo, or in situ. For example, in situ polymerizable gels can be used in accordance with the invention. Examples of such gels are known in the art.

In some embodiments, the cells provided herein are seeded onto such three-dimensional matrices, such as scaffolds and implanted in vivo, where the seeded cells may proliferate on or in the framework or help establish replacement tissue in vivo with or without cooperation of other cells. Growth of the amnion derived adherent cells or co-cultures thereof on the three-dimensional framework preferably results in the formation of a three-dimensional tissue, or foundation thereof, which can be utilized in vivo, for example for repair of damaged or diseased tissue. For example, the three-dimensional scaffolds can be used to form tubular structures, for example for use in repair of blood vessels; or aspects of the circulatory system or coronary structures. In accordance with one aspect of the invention, amnion derived adherent cells, or co-cultures thereof, are inoculated, or seeded on a three-dimensional framework or matrix, such as a scaffold, a foam or hydrogel. The framework may be configured into various shapes such as generally flat, generally cylindrical or tubular, or can be completely free-form as may be required or desired for the corrective structure under consideration. In some embodiments, the amnion derived adherent cells grow on the three dimensional structure, while in other embodiments, the cells only survive, or even die, but stimulate or promote ingrowth of new tissue or vascularization in a recipient.

The cells of the invention can be grown freely in culture, removed from the culture and inoculated onto a three-dimensional framework. Inoculation of the three-dimensional framework with a concentration of cells, e.g., approximately 10⁶ to 5×10⁷ cells per milliliter, preferably results in the establishment of the three-dimensional support in relatively shorter periods of time. Moreover in some application it may be preferably to use a greater or lesser number of cells depending on the result desired.

In a specific embodiment, the matrix can be cut into a strip (e.g., rectangular in shape) of which the width is approximately equal to the inner circumference of a tubular organ into which it will ultimately be inserted. The amnion derived adherent cells can be inoculated onto the scaffold and incubated by floating or suspending in liquid media. At the appropriate stage of confluence, the scaffold can be rolled up into a tube by joining the long edges together. The seam can then be closed by suturing the two edges together using fibers of a suitable material of an appropriate diameter. In order to prevent cells from occluding the lumen, one of the open ends of the tubular framework can be affixed to a nozzle. Liquid media can be forced through the nozzle from a source chamber connected to the incubation chamber to create a current through the interior of the tubular framework. The other open end can be affixed to an outflow aperture which leads into a collection chamber from which the media can be recirculated through the source chamber. The tube can be detached from the nozzle and outflow aperture when incubation is complete. See, e.g., International Application No. WO 94/25584.

In general, two three-dimensional frameworks can be combined into a tube in accordance with the invention using any of the following methods. Two or more flat frameworks can be laid atop another and sutured together. The resulting two-layer sheet can then be rolled up, and, as described above, joined together and secured. In certain embodiments, one tubular scaffold that is to serve as the inner layer can be inoculated with amnion derived adherent cells and incubated. A second scaffold can be grown as a flat strip with width slightly larger than the outer circumference of the tubular framework. After appropriate growth is attained, the flat framework is wrapped around the outside of the tubular scaffold followed by closure of the seam of the two edges of the flat framework and securing the flat framework to the inner tube. In another embodiment, two or more tubular meshes of slightly differing diameters can be grown separately. The framework with the smaller diameter can be inserted inside the larger one and secured. For each of these methods, more layers can be added by reapplying the method to the double-layered tube. The scaffolds can be combined at any stage of growth of the amnion derived adherent cells, and incubation of the combined scaffolds can be continued when desirable.

In conjunction with the above, the cells and therapeutic compositions provided herein can be used in conjunction with implantable devices. For example the amnion derived adherent cells can be coadminstered with, for example, stents, artificial valves, ventricular assist devices, Guglielmi detachable coils and the like. As the devices may constitute the dominant therapy provided to an individual in need of such therapy, the cells and the like may be used as supportive or secondary therapy to assist in, stimulate, or promote proper healing in the area of the implanted device. The cells and therapeutic compositions of the invention may also be used to pretreat certain implantable devices, to minimize problems when they are used in vivo. Such pretreated devices, including coated devices, may be better tolerated by patients receiving them, with decrease risk of local or systemic infection, or for example, restenosis or further occlusion of blood vessels.

5.9.2 Media Conditioned by Amnion Derived Adherent Cells

Further provided herein is medium that has been conditioned by amnion derived adherent cells, that is, medium comprising one or more biomolecules secreted or excreted by the adherent cells. In various embodiments, the conditioned medium comprises medium in which the cells have grown for at least 1, 2, 3, 4, 5, 6, 7, 8, 9, 10, 11, 12, 13, 14 or more days, or for at least 1, 2, 3, 4, 5, 6, 7, 8, 9, 10, 11, 12, 13, 14, 15, 16, 17, 18, 19, or 20 population doublings, or more. In other embodiments, the conditioned medium comprises medium in which amnion derived adherent cells have grown to at least 30%, 40%, 50%, 60%, 70%, 80%, 90% confluence, or up to 100% confluence. Such conditioned medium can be used to support the culture of a population of cells, e.g., stem cells, e.g., placental stem cells, embryonic stem cells, embryonic germ cells, adult stem cells, or the like. In another embodiment, the conditioned medium comprises medium in which amnion derived adherent cells, and cells that are not amnion derived adherent cells, have been cultured together.

The conditioned medium can comprise the adherent cells provided herein. Thus, provided herein is a cell culture comprising amnion derived adherent cells. In a specific embodiment, the conditioned medium comprises a plurality, e.g., a population, of amnion derived adherent cells.

### 5.10 Modified Amnion Derived Adherent Cells

5.10.1 Genetically Modified Amnion Derived Adherent Cells

In another aspect, the amnion derived adherent cells described herein can be genetically modified, e.g., to produce a nucleic acid or polypeptide of interest, or to produce a differentiated cell, e.g., an osteogenic cell, myocytic cell, pericytic cell, or angiogenic cell, that produces a nucleic acid or polypeptide of interest. For example, the amnion derived adherent cells can be modified to produce angiogenic factors, such as proangiogenic molecules, soluble factors and receptors or promigratory molecules such as chemokines, e.g., stromal cell derived factor 1 (SDF-1) or chemokine receptors. Genetic modification can be accomplished, e.g., using virus-based vectors including, but not limited to, non-integrating replicating vectors, e.g., papilloma virus vectors, SV40 vectors, adenoviral vectors; integrating viral vectors, e.g., retrovirus vector or adeno-associated viral vectors; or replication-defective viral vectors. Other methods of introducing DNA into cells include the use of liposomes, electroporation, a particle gun, direct DNA injection, or the like.

The adherent cells provided herein can be, e.g., transformed or transfected with DNA controlled by or in operative association with, one or more appropriate expression control elements, for example, promoter or enhancer sequences, transcription terminators, polyadenylation sites, internal ribosomal entry sites. Preferably, such a DNA incorporates a selectable marker. Following the introduction of the foreign DNA, engineered adherent cells can be, e.g., grown in enriched media and then switched to selective media. In one embodiment, the DNA used to engineer a amnion derived adherent cell comprises a nucleotide sequence encoding a polypeptide of interest, e.g., a cytokine, growth factor, differentiation agent, or therapeutic polypeptide.

The DNA used to engineer the adherent cell can comprise any promoter known in the art to drive expression of a nucleotide sequence in mammalian cells, e.g., human cells. For example, promoters include, but are not limited to, CMV promoter/enhancer, SV40 promoter, papillomavirus promoter, Epstein-Barr virus promoter, elastin gene promoter, and the like. In a specific embodiment, the promoter is regulatable so that the nucleotide sequence is expressed only when desired. Promoters can be either inducible (e.g., those associated with metallothionein and heat shock proteins) or constitutive.

In another specific embodiment, the promoter is tissue-specific or exhibits tissue specificity. Examples of such promoters include but are not limited to myosin light chain-2 gene control region (Shani, 1985, Nature 314:283) (skeletal muscle).

The amnion derived adherent cells disclosed herein may be engineered or otherwise selected to “knock out” or “knock down” expression of one or more genes in such cells. The expression of a gene native to a cell can be diminished by, for example, inhibition of expression by inactivating the gene completely by, e.g., homologous recombination. In one embodiment, for example, an exon encoding an important region of the protein, or an exon 5′ to that region, is interrupted by a positive selectable marker, e.g., neo, preventing the production of normal mRNA from the target gene and resulting in inactivation of the gene. A gene may also be inactivated by creating a deletion in part of a gene or by deleting the entire gene. By using a construct with two regions of homology to the target gene that are far apart in the genome, the sequences intervening the two regions can be deleted (Mombaerts et al., 1991, Proc. Nat. Acad. Sci. U.S.A. 88:3084). Antisense, morpholinos, DNAzymes, small interfering RNA, short hairpin RNA, and ribozyme molecules that inhibit expression of the target gene can also be used to reduce the level of target gene activity in the adherent cells. For example, antisense RNA molecules which inhibit the expression of major histocompatibility gene complexes (HLA) have been shown to be most versatile with respect to immune responses. Triple helix molecules can be utilized in reducing the level of target gene activity. See, e.g., L. G. Davis et al. (eds), 1994, BASIC METHODS IN MOLECULAR BIOLOGY, 2nd ed., Appleton & Lange, Norwalk, Conn., which is incorporated herein by reference.

In a specific embodiment, the amnion derived adherent cells disclosed herein can be genetically modified with a nucleic acid molecule comprising a nucleotide sequence encoding a polypeptide of interest, wherein expression of the polypeptide of interest is controllable by an exogenous factor, e.g., polypeptide, small organic molecule, or the like. The polypeptide of interest can be a therapeutic polypeptide. In a more specific embodiment, the polypeptide of interest is IL-12 or interleukin-1 receptor antagonist (IL-1Ra). In another more specific embodiment, the polypeptide of interest is a fusion of interleukin-1 receptor antagonist and dihydrofolate reductase (DHFR), and the exogenous factor is an antifolate, e.g., methotrexate. Such a construct is useful in the engineering of amnion derived adherent cells that express IL-1Ra, or a fusion of IL-1Ra and DHFR, upon contact with methotrexate. Such a construct can be used, e.g., in the treatment of rheumatoid arthritis. In this embodiment, the fusion of IL-1Ra and DHFR is translationally upregulated upon exposure to an antifolate such as methotrexate. Therefore, in another specific embodiment, the nucleic acid used to genetically engineer an amnion derived adherent cell can comprise nucleotide sequences encoding a first polypeptide and a second polypeptide, wherein said first and second polypeptides are expressed as a fusion protein that is translationally upregulated in the presence of an exogenous factor. The polypeptide can be expressed transiently or long-term (e.g., over the course of weeks or months). Such a nucleic acid molecule can additionally comprise a nucleotide sequence encoding a polypeptide that allows for positive selection of engineered cells, or allows for visualization of the engineered cells. In another more specific embodiment, the nucleotide sequence encodes a polypeptide that is, e.g., fluorescent under appropriate visualization conditions, e.g., luciferase (Luc). In a more specific embodiment, such a nucleic acid molecule can comprise IL-1Ra-DHFR-IRES-Luc, where IL-1Ra is interleukin-1 receptor antagonist, IRES is an internal ribosomal entry site, and DHFR is dihydrofolate reductase.

5.10.2 Immortalized Amnion Derived Adherent Cell Lines

Mammalian amnion derived adherent cells can be conditionally immortalized by transfection with any suitable vector containing a growth-promoting gene, that is, a gene encoding a protein that, under appropriate conditions, promotes growth of the transfected cell, such that the production and/or activity of the growth-promoting protein is regulatable by an external factor. In a preferred embodiment the growth-promoting gene is an oncogene such as, but not limited to, v-myc, N-myc, c-myc, p53, SV40 large T antigen, polyoma large T antigen, E1a adenovirus or E7 protein of human papillomavirus. In another embodiment, amnion derived adherent cells can be immortalized using cre-lox recombination, as exemplified for a human pancreatic β-cell line by Narushima, M., et at (Nature Biotechnology, 2005, 23(10:1274-1282).

External regulation of the growth-promoting protein can be achieved by placing the growth-promoting gene under the control of an externally-regulatable promoter, e.g., a promoter the activity of which can be controlled by, for example, modifying the temperature of the transfected cells or the composition of the medium in contact with the cells. in one embodiment, a tetracycline (tet)-controlled gene expression system can be employed (see Gossen et al., Proc. Natl. Acad. Sci. USA 89:5547-5551, 1992; Hoshimaru et al., Proc. Natl. Acad. Sci. USA 93:1518-1523, 1996). In the absence of tet, a tet-controlled transactivator (tTA) within this vector strongly activates transcription from ph_{CMV*-1}, a minimal promoter from human cytomegalovirus fused to tet operator sequences. tTA is a fusion protein of the repressor (tetR) of the transposon-10-derived tet resistance operon of Escherichia coli and the acidic domain of VP16 of herpes simplex virus. Low, non-toxic concentrations of tet (e.g., 0.01-1.0 μg/mL) almost completely abolish transactivation by tTA.

In one embodiment, the vector further contains a gene encoding a selectable marker, e.g., a protein that confers drug resistance. The bacterial neomycin resistance gene (neo^{R}) is one such marker that may be employed within the present methods. Cells carrying neo^{R} may be selected by means known to those of ordinary skill in the art, such as the addition of, e.g., 100-200 μg/mL G418 to the growth medium.

Transfection can be achieved by any of a variety of means known to those of ordinary skill in the art including, but not limited to, retroviral infection. In general, a cell culture may be transfected by incubation with a mixture of conditioned medium collected from the producer cell line for the vector and DMEM/F12 containing N2 supplements. For example, a placental cell culture prepared as described above may be infected after, e.g., five days in vitro by incubation for about 20 hours in one volume of conditioned medium and two volumes of DMEM/F12 containing N2 supplements. Transfected cells carrying a selectable marker may then be selected as described above.

Following transfection, cultures are passaged onto a surface that permits proliferation, e.g., allows at least 30% of the cells to double in a 24 hour period. Preferably, the substrate is a polyornithine/laminin substrate, consisting of tissue culture plastic coated with polyornithine (10 μg/mL) and/or laminin (10 μg/mL), a polylysine/laminin substrate or a surface treated with fibronectin. Cultures are then fed every 3-4 days with growth medium, which may or may not be supplemented with one or more proliferation-enhancing factors. Proliferation-enhancing factors may be added to the growth medium when cultures are less than 50% confluent.

The conditionally-immortalized amnion derived adherent cell lines can be passaged using standard techniques, such as by trypsinization, when 80-95% confluent. Up to approximately the twentieth passage, it is, in some embodiments, beneficial to maintain selection (by, for example, the addition of G418 for cells containing a neomycin resistance gene). Cells may also be frozen in liquid nitrogen for long-term storage.

Clonal cell lines can be isolated from a conditionally-immortalized adherent cell line prepared as described above. In general, such clonal cell lines may be isolated using standard techniques, such as by limit dilution or using cloning rings, and expanded. Clonal cell lines may generally be fed and passaged as described above.

Conditionally-immortalized human amnion derived adherent cells lines, which may, but need not, be clonal, may generally be induced to differentiate by suppressing the production and/or activity of the growth-promoting protein under culture conditions that facilitate differentiation. For example, if the gene encoding the growth-promoting protein is under the control of an externally-regulatable promoter, the conditions, e.g., temperature or composition of medium, may be modified to suppress transcription of the growth-promoting gene. For the tetracycline-controlled gene expression system discussed above, differentiation can be achieved by the addition of tetracycline to suppress transcription of the growth-promoting gene. In general, 1 μg/mL tetracycline for 4-5 days is sufficient to initiate differentiation. To promote further differentiation, additional agents may be included in the growth medium.

### 5.11 Dosages and Routes of Administration

Administration of AMDACs to an individual in need thereof can be by any medically-acceptable route relevant for the disease or condition to be treated. In another specific embodiment of the methods of treatment described above, said AMDACs are administered by bolus injection. In another specific embodiment, said isolated AMDACs are administered by intravenous infusion. In a specific embodiment, said intravenous infusion is intravenous infusion over about 1 to about 8 hours. In another specific embodiment, said isolated AMDACs are administered intracranially. In another specific embodiment, said isolated AMDACs are administered intramuscularly. In another specific embodiment, said isolated AMDACs are administered intraperitoneally. In another specific embodiment, said isolated AMDACs are administered intra-arterially. In a more specific embodiment, said isolated AMDACs are administered within an area of ischemia. In another more specific embodiment, said isolated AMDACs are administered to an area peripheral to an ischemia. In another specific embodiment of the method of treatment, said isolated AMDACs are administered intramuscularly, intradermally, or subcutaneously.

In another specific embodiment of the methods of treatment described above, said AMDACs are administered once to said individual. In another specific embodiment, said isolated AMDACs are administered to said individual in two or more separate administrations. In another specific embodiment, said administering comprises administering between about 1×10⁴ and 1×10⁵ isolated AMDACs, e.g., AMDACs per kilogram of said individual. In another specific embodiment, said administering comprises administering between about 1×10⁵ and 1×10⁶ isolated AMDACs per kilogram of said individual. In another specific embodiment, said administering comprises administering between about 1×10⁶ and 1×10⁷ isolated AMDACs per kilogram of said individual. In another specific embodiment, said administering comprises administering between about 1×10⁷ and 1×10⁸ isolated placental cells per kilogram of said individual. In other specific embodiments, said administering comprises administering between about 1×10⁶ and about 2×10⁶ isolated placental cells per kilogram of said individual; between about 2×10⁶ and about 3×10⁶ isolated placental cells per kilogram of said individual; between about 3×10⁶ and about 4×10⁶ isolated placental cells per kilogram of said individual; between about 4×10⁶ and about 5×10⁶ isolated placental cells per kilogram of said individual; between about 5×10⁶ and about 6×10⁶ isolated placental cells per kilogram of said individual; between about 6×10⁶ and about 7×10⁶ isolated placental cells per kilogram of said individual; between about 7×10⁶ and about 8×10⁶ isolated placental cells per kilogram of said individual; between about 8×10⁶ and about 9×10⁶ isolated placental cells per kilogram of said individual; or between about 9×10⁶ and about 1×10⁷ isolated placental cells per kilogram of said individual. In another specific embodiment, said administering comprises administering between about 1×10⁷ and about 2×10⁷ isolated placental cells per kilogram of said individual to said individual. In another specific embodiment, said administering comprises administering between about 1.3×10⁷ and about 1.5×10⁷ isolated placental cells per kilogram of said individual to said individual. In another specific embodiment, said administering comprises administering up to about 3×10⁷ isolated placental cells per kilogram of said individual to said individual. In a specific embodiment, said administering comprises administering between about 5×10⁶ and about 2×10⁷ isolated placental cells to said individual. In another specific embodiment, said administering comprises administering about 150×10⁶ isolated placental cells in about 20 milliliters of solution to said individual.

In a specific embodiment, said administering comprises administering between about 5×10⁶ and about 2×10⁷ isolated placental cells to said individual, wherein said cells are contained in a solution comprising 10% dextran, e.g., dextran-40, 5% human serum albumin, and optionally an immunosuppressant. In another specific embodiment, said administering comprises administering between about 5×10⁷ and 3×10⁹ isolated placental cells intravenously. In more specific embodiments, said administering comprises administering about 9×10⁸ isolated placental cells or about 1.8×10⁹ isolated placental cells intravenously. In another specific embodiment, said administering comprises administering between about 5×10⁷ and 1×10⁸ isolated placental cells intracranially. In a more specific embodiment, said administering comprises administering about 9×10⁷ isolated placental cells intracranially.

### 5.12 Differentiation of Amnion Derived Adherent Cells

The amnion derived adherent cells provided herein can be differentiated. In one embodiment, the cell has been differentiated sufficiently for said cell to exhibit at least one characteristic of an endothelial cell, a myogenic cell, or a pericytic cell, e.g., by contacting the cell with vascular endothelial growth factor (VEGF). In more specific embodiments, said characteristic of an endothelial cell, myogenic cell or pericytic cell is expression of one or more of CD9, CD31, CD54, CD102, NG2 (neural/glial antigen 2) or alpha smooth muscle actin, which is increased compared to an amniotic cell that is OCT-4⁻, VEGFR2/KDR⁺, CD9⁺, CD54⁺, CD105⁺, CD200⁺, and VE-cadherin⁻. In other more specific embodiments, said characteristic of an endothelial cell, myogenic cell or pericytic cell is expression of one or more of CD9, CD31, CD54, CD102, NG2 (neural/glial antigen 2) or alpha smooth muscle actin, which is increased compared to an amniotic cell that is OCT-4⁻, VEGFR2/KDR⁺, and VEGFR1/Flt-1⁺.

5.12.1 Induction of Angiogenesis

Angiogenesis from the amnion derived adherent cells provided herein can be accomplished as follows. The amnion derived adherent cells, are cultured, e.g., in an endothelial cell medium, e.g., EGM®-2 (Lonza) or a medium comprising 60% DMEM-LG (Gibco), 40% MCDB-201 (Sigma); 2% fetal calf serum (Hyclone Labs.); 1× insulin-transferrin-selenium (ITS); 1× linoleic acid-bovine serum albumin (LA-BSA); 5×10⁻⁹ M dexamethasone (Sigma); 10⁻⁴ M ascorbic acid 2-phosphate (Sigma); epidermal growth factor 10 ng/mL (R&D Systems); and platelet-derived growth factor (PDGF-BB) 10 ng/mL (R&D Systems), to passage 3. The cells are then plated onto MATRIGEL™ or a substrate comprising collagen-1, e.g., in 96-well plates at a density of, e.g., about 1.5×10⁴ cells per well in the same medium or DMEM with FBS (0-5% v/v) comprising vascular endothelial growth factor (VEGF) at, e.g., about 10 to 50 ng per milliliter. Medium can be changed about twice a week. Angiogenesis is evidenced by visual inspection of the cells for sprouting of vessel-like structures and tube formation, visible under a microscope at a magnification of, e.g., 50× to 100×.

5.12.2 Induction of Differentiation into Cardiac Cells

Myogenic (cardiogenic) differentiation of the amnion derived adherent cells provided herein can be accomplished, for example, by placing the cells in cell culture conditions that induce differentiation into cardiomyocytes. A preferred cardiomyocytic medium comprises DMEM/20% CBS supplemented with retinoic acid, 1 μM; basic fibroblast growth factor, 10 ng/mL; and transforming growth factor beta-1, 2 ng/mL; and epidermal growth factor, 100 ng/mL. KnockOut Serum Replacement (Invitrogen, Carlsbad, Calif.) may be used in lieu of CBS. Alternatively, the amnion derived adherent cells are cultured in DMEM/20% CBS supplemented with 1 to 100, e.g., 50 ng/mL Cardiotropin-1 for 24 hours. In another embodiment, amnion derived adherent cells can be cultured 10-14 days in protein-free medium for 5-7 days, then stimulated with human myocardium extract, e.g., produced by homogenizing human myocardium in 1% HEPES buffer supplemented with 1% cord blood serum.

Differentiation can be confirmed by demonstration of cardiac actin gene expression, e.g., by RT/PCR, or by visible beating of the cell. An adherent cell is considered to have differentiated into a cardiac cell when the cell displays one or more of these characteristics.

### 6. EXAMPLES

### 6.1 Example 1

### Isolation and Expansion of Adherent Cells from Amniotic Membrane

This Example demonstrates the isolation and expansion of amnion derived adherent cells.

6.1.1 Isolation

Amnion derived adherent cells were isolated from amniotic membrane as follows. Amnion/chorion were cut from the placenta, and amnion was manually separated from the chorion. The amnion was rinsed with sterile PBS to remove residual blood, blood clots and other material. Sterile gauze was used to remove additional blood, blood clots or other material that was not removed by rinsing, and the amnion was rinsed again with PBS. Excess PBS was removed from the membrane, and the amnion was cut with a scalpel into 2″ by 2″ segments. For epithelial cell release, a processing vessel was set up by connecting a sterile jacketed glass processing vessel to a circulating 37° C. water bath using tubing and connectors, and set on a stir plate. Trypsin (0.25%, 300 mL) was warmed to 37° C. in the processing vessel; the amnion segments were added, and the amnion/trypsin suspension was agitated, e.g., at 100 RPM-150 RPM at 37° C. for 15 minutes. A sterile screening system was assembled by placing a sterile receptacle on a sterile field next to the processing vessel and inserting a sterile 75 μm to 125 μm screen into the receptacle (Millipore, Billerica, Mass.). After agitating the amnion segments for 15 minutes, the contents of the processing vessel were transferred to the screen, and the amnion segments were transferred, e.g., using sterile tweezers back into the processing vessel; the trypsin solution containing the epithelial cells was discarded. The amnion segments were agitated again with 300 mL trypsin solution (0.25%) as described above. The screen was rinsed with approximately 100-150 mL of PBS, and the PBS solution was discarded. After agitating the amnion segments for 15 minutes, the contents of the processing vessel were transferred to the screen. The amnion segments were then transferred back into the processing vessel; the trypsin solution containing the epithelial cells was discarded. The amnion segments were agitated again with 300 mL trypsin solution (0.25%) as described above. The screen was rinsed with approximately 100-150 mL of PBS, and the PBS solution was discarded. After agitating the amnion segments for 15 minutes, the contents of the processing vessel were transferred to the screen. The amnion segments were then transferred back into the processing vessel, and the trypsin solution containing the epithelial cells was discarded. The amnion segments were agitated in PBS/5% FBS (1:1 ratio of amnion to PBS/5% FBS solution by volume) at 37° C. for approximately 2-5 minutes to neutralize the trypsin. A fresh sterile screen system was assembled. After neutralizing the trypsin, the contents of the processing vessel were transferred to the new screen, and the amnion segments were transferred back into the processing vessel. Room temperature, sterile PBS (400 mL) was added to the processing vessel, and the contents of the processing vessel were agitated for approximately 2-5 minutes. The screen was rinsed with approximately 100-150 mL of PBS. After agitation, the contents of the processing vessel were transferred to the screen; the processing flask was rinsed with PBS, and the PBS solution was discarded. The processing vessel was then filled with 300 mL of pre-warmed DMEM, and the amnion segments were transferred into the DMEM solution.

For release of the amnion derived adherent cells, the treated amniotic membrane was further treated with collagenase as follows. A sterile collagenase stock solution (500 U/mL) was prepared by dissolving the appropriate amount of collagenase powder (varied with the activity of the collagenase lot received from the supplier) in DMEM. The solution was filtered through a 0.22 μm filter and dispensed into individual sterile containers. CaCl₂ solution (0.5 mL, 600 mM) was added to each 100 mL dose, and the doses were frozen. Collagenase (100 mL) was added to the amnion segments in the processing vessel, and the processing vessel was agitated for 30-50 minutes, or until amnion digestion was complete by visual inspection. After amnion digestion was complete, 100 mL of pre-warmed sterile PBS/5% FBS was added to the processing vessel, and the processing vessel was agitated for an additional 2-3 minutes. Following agitation, the contents of the flask were transferred to a sterile 60 μm screen, and the liquid was collected by vacuum filtration. The processing vessel was rinsed with 400 mL of PBS, and the PBS solution was sterile-filtered. The filtered cell suspension was then centrifuged at 300×g for 15 minutes at 20° C., and the cell pellets were resuspended in pre-warmed PBS/2% FBS (approximately 10 mL total).

6.1.2 Establishment

Freshly isolated angiogenic amniotic cells were added to growth medium containing 60% DMEM-LG (Gibco); 40% MCBD-201 (Sigma); 2% FBS (Hyclone Labs), 1× insulin-transferrin-selenium (ITS); 10 ng/mL linoleic acid-bovine serum albumin (LA-BSA); 1 n-dexamethasone (Sigma); 100 μM ascorbic acid 2-phosphate (Sigma); 10 ng/mL epidermal growth factor (R & D Systems); and 10 ng/mL platelet-derived growth factor (PDGF-BB) (R & D Systems) and were plated in a T-Flask at a seeding density of 10,000 cells per cm². The culture device(s) were then incubated at 37° C., 5% CO₂ with >90% humidity. Cellular attachment, growth, and morphology were monitored daily. Non-adherent cells and debris were removed by medium exchange. Medium exchange was performed twice per week. Adherent cells with typical fibroblastoid/spindle shape morphology appeared at several days after initial plating. When confluency reached 40%-70% (at 4-11 days after initial plating), the cells were harvested by trypsinization (0.25% trypsin—EDTA) for 5 minutes at room temperature (37° C.). After neutralization with PBS-5% FBS, the cells were centrifuged at 200-400 g for 5-15 minutes at room temperature, and then were resuspended in growth medium. At this point, an AMDACS line was considered to be successfully established at the initial passage. Initial passage amnion derived adherent cells were, in some cases, cryopreserved or expanded.

6.1.3 Culture Procedure

Amnion derived adherent cells were cultured in the growth medium described above and seeded at a density of 2000-4000 per cm² in an appropriate tissue culture—treated culture device(s). The culture device(s) were then incubated at 37° C., 5% CO₂ with >90% humidity. During culture, AMDACs would adhere and proliferate. Cellular growth, morphology, and confluency were monitored daily. Medium exchange was performed twice a week to replenish fresh nutrients if the culture extended to 5 days or more. When confluency reached 40%-70% (at 3-7 days after seeding), the cells were harvested by trypsinization (0.05%-0.25% trypsin—EDTA) for 5 minutes at room temperature (37° C.). After neutralization with PBS-5% FBS, the cells were centrifuged at 200-400 g for 5-15 minutes at room temperature, then were resuspended in growth medium.

AMDACs isolated and cultured in this manner typically produced 33530+/−15090 colony-forming units (fibroblast) (CFU-F) out of 1×10⁶ cells plated.

### 6.2 Example 2

### Phenotypic Characterization of Amnion Derived Adherent Cells

6.2.1 Gene and Protein Expression Profiles

This Example describes phenotypic characterization of amnion derived adherent cells, including characteristic cell surface marker, mRNA, and proteomic expression.

Sample Preparation:

Amnion derived adherent cells were obtained as described in Example 1. The cells at passage 6 were grown to approximately 70% confluence in growth medium as described in Example 1, above, trypsinized, and washed in PBS. NTERA-2 cells (American Type Culture Collection, ATCC Number CRL-1973) were grown in DMEM containing 4.5 g/L glucose, 2 mM glutamine and 10% FBS. Nucleated cell counts were performed to obtain a minimum of 2×10⁶ to 1×10⁷ cells. The cells were then lysed using a Qiagen RNeasy kit (Qiagen, Valencia, Calif.), utilizing a QlAshredder, to obtain the lysates. The RNA isolation was then performed using a Qiagen RNeasy kit. RNA quantity and quality were determined using a Nanodrop ND 1000 spectrophotometer, 25 ng/μL of RNA/reaction. The cDNA reactions were prepared using an Applied Biosystems (Foster City, Calif.) High Capacity cDNA Archive Kit. Real time PCR reactions were performed using TAQMAN® universal PCR master mixes from Applied Biosystems. Reactions were run in standard mode on an Applied Biosystems 7300 Real time PCR system for 40 cycles.

Sample Analysis and Results:

Using the real time PCR methodology and specific TAQMAN® gene expression probes and/or the TAQMAN® human angiogenesis array (Applied Biosystems), cells were characterized for expression of stem cell-related, angiogenic and cardiomyogenic markers. Results were expressed either as the relative expression of a gene of interest in comparison to the pertinent cell controls, or the relative expression (delta Ct) of the gene of interest in comparison to a ubiquitously expressed housekeeping gene (for example, GAPDH, 18S, or GUSB).

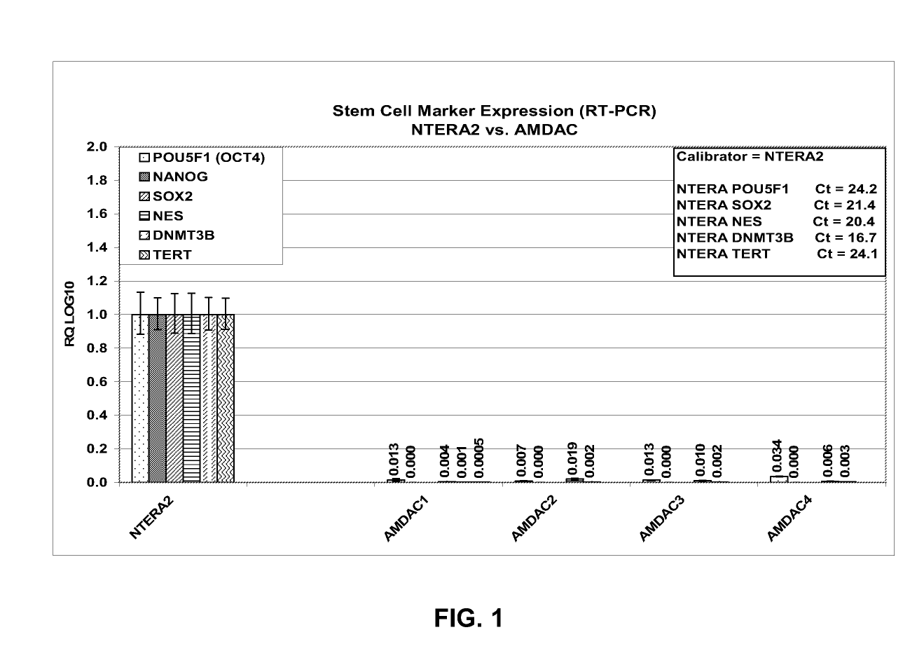

Amnion derived adherent cells expressed various, stem-cell related, angiogenic and cardiomyogenic genes and displayed a relative absence of OCT-4 expression in comparison to NTERA-2 cells. Table 1 summarizes the expression of selected angiogenic, cardiomyogenic, and stem cell genes, and FIG. 1 demonstrates the lack of expression in AMDACs of the stem cell-related genes POU5F1 (OCT-4), NANOG, SOX2, NES, DNMT3B, and TERT.

TABLE 1
Gene expression profile of amnion derived adherent cells
as determined by RT-PCR.

| AMDACSMarker | Positive | Negative | mRNA |
|---|---|---|---|
| ACTA2 | X | | X |
| ACTC1 | X | | X |
| ADAMTS1 | X | | X |
| AMOT | X | | X |
| ANG | X | | X |
| ANGPT1 | X | | X |
| ANGPT2 | X | | X |
| ANGPT4 | | X | X |
| ANGPTL1 | X | | X |
| ANGPTL2 | X | | X |
| ANGPTL3 | | X | X |
| ANGPTL4 | X | | X |
| BAI1 | X | | X |
| BGLAP | | X | X |
| c-myc | X | | X |
| CD31 | | X | X |
| CD34 | | X | X |
| CD44 | X | | X |
| CD140a | X | | X |
| CD140b | X | | X |
| CD200 | X | | X |
| CD202b | X | | X |
| CD304 | X | | X |
| CD309 | X | | X |
| (VEGFR2/KDR) | | | |
| CDH5 | | X | X |
| CEACAM1 | X | | X |
| CHGA | X | | X |
| COL15A1 | X | | X |
| COL18A1 | X | | X |
| COL4A1 | X | | X |
| COL4A2 | X | | X |
| COL4A3 | X | | X |
| Connexin-43 | X | | X |
| CSF3 | X | | X |
| CTGF | X | | X |
| CXCL10 | | X | X |
| CXCL12 | X | | X |
| CXCL2 | X | | X |
| DLX5 | | X | X |
| DNMT3B | X | | X |
| ECGF1 | X | | X |
| EDG1 | X | | X |
| EDIL3 | X | | X |
| ENPP2 | X | | X |
| EPHB2 | X | | X |
| F2 | X | | X |
| FBLN5 | X | | X |
| FGA | | X | X |
| FGF1 | X | | X |
| FGF2 | X | | X |
| FGF4 | | X | X |
| FIGF | X | | X |
| FLT3 | | X | X |
| FLT4 | X | | X |
| FN1 | X | | X |
| FOXC2 | X | | X |
| Follistatin | X | | X |
| Galectin-1 | X | | X |
| GRN | X | | X |
| HEY1 | X | | X |
| HGF | X | | X |
| HLA-G | | X | X |
| HSPG2 | X | | X |
| IFNB1 | X | | X |
| IFNG | | X | X |
| IL-8 | X | | X |
| IL-12A | X | | X |
| ITGA4 | X | | X |
| ITGAV | X | | X |
| ITGB3 | X | | X |
| KLF-4 | X | | X |
| LECT1 | | X | X |
| LEP | | X | X |
| MDK | X | | X |
| MMP-13 | | X | X |
| MMP-2 | X | | X |
| MYOZ2 | X | | X |
| NANOG | | X | X |
| NESTIN | | X | X |
| NRP2 | X | | X |
| PDGFB | X | | X |
| PF4 | X | | X |
| PGK1 | X | | X |
| PLG | | X | X |
| POU5F1 (OCT-4) | | X | X |
| PRL | | X | X |
| PROK1 | | X | X |
| PROX1 | X | | X |
| PTN | X | | X |
| SEMA3F | X | | X |
| SERPINB5 | X | | X |
| SERPINC1 | X | | X |
| SERPINF1 | X | | X |
| SOX2 | | X | X |
| TERT | | X | X |
| TGFA | X | | X |
| TGFB1 | X | | X |
| THBS1 | X | | X |
| THBS2 | X | | X |
| TIE1 | X | | X |
| TIMP2 | X | | X |
| TIMP3 | X | | X |
| TNF | X | | X |
| TNFSF15 | X | | X |
| TNMD | | X | X |
| TNNC1 | X | | X |
| TNNT2 | X | | X |
| VASH1 | X | | X |
| VEGF | X | | X |
| VEGFB | X | | X |
| VEGFC | X | | X |
| VEGFR1/FLT-1 | X | | X |
| XLKD1 | | X | X |

Column “mRNA” indicates that the presence or absence of mRNA for particular markers were determined in each instance.

In a separate experiment, AMDACs were additionally found to express genes for Aryl hydrocarbon receptor nuclear translocator 2 (ARNT2), nerve growth factor (NGF), brain-derived neurotrophic factor (BDNF), glial-derived neurotrophic factor (GDNF), neurotrophin 3 (NT-3), NT-5, hypoxia-Inducible Factor 1α (HIF1A), hypoxia-inducible protein 2 (HIG2), heme oxygenase (decycling) 1 (HMOX1), Extracellular superoxide dismutase [Cu—Zn] (SOD3), catalase (CAT), transforming growth factor β1 (TGFB1), transforming growth factor β1 receptor (TGFB1R), and hepatoycte growth factor receptor (HGFR/c-met).

6.2.2 Flow Cytometry for Evaluation of Angiogenic Potency of Amnion Derived Adherent Cells

Flow cytometry was used as a method to quantify phenotypic markers of amnion derived adherent cells to define the identity of the cells. Cell samples were obtained from frozen stocks. Prior to thaw and during reagent preparation, cell vials were maintained on dry ice. Subsequently, samples were thawed rapidly using a 37° C. water bath. Pre-freeze cell counts were used for calculations for initial post-thaw cell number-dependent dilutions. Briefly, cryovials were thawed in a 37° C. water bath for approximately 30 seconds with gentle agitation. Immediately following thawing, approximately 100-200 μL of cold (2 to 8° C.) thawing solution (PBS with 2.5% albumin and 5% Gentran 40) was added to the cryovial and mixed. After gentle mixing, the total volume in the cryovials was transferred into a 15 mL conical tube containing an equal volume of cold (2 to 8° C.) thawing solution. The cells were centrifuged in a conical tube at 400 g for 5 minutes at room temperature before removing the supernatant. The residual volume was measured with a pipette (estimation); the residual volume and cell pellet were resuspended at room temperature in 1% FBS in PBS to achieve a cell concentration of 250×10³ cells/100 μL buffer. For example, 1×10⁶ cells would be resuspended in 400 μL 1% FBS. The cell suspension was placed into pre-labeled 5 mL FACS tubes (Becton Dickinson (BD), Franklin Lakes, N.J.). For each primary antibody isotype, 100 μL of cell suspension was aliquoted into one isotype control tube. Prior to phenotype analysis, the concentrations of all antibodies were optimized to achieve good signal to noise ratios and adequate detection of CD antigens across a potential four-log dynamic range. The volume of each isotype and sample antibody that was used to stain each sample was determined. To standardize the amount of antibody (in μg) in the isotype and sample tubes, the concentration of each antibody was calculated as (1/actual antibody concentration (μg/μL))×(desired final quantity of antibody in μg for 2.5×10⁵ cells)=#μL of antibody added. A master mix of antibodies for both the isotype and the sample was made with the appropriate amount of antibody added to each tube. The cells were stained for 15-20 minutes at room temperature in the dark. After staining, unbound antibody in each sample was removed by centrifugation (400 g×5 minutes) followed by washing using 2 mL 1% FBS PBS (room temperature) before resuspension in 150 μL of room temperature 1% FBS PBS. The samples were then analyzed on Becton Dickinson FACSCalibur, FACSCantoI or BD FACSCantoII flow cytometers prepared for use per manufacturer's instructions. Multi-parametric flow cytometry data sets (side scatter (SSC), forward scatter (FSC) and integrated fluorescence profiles (FL)) were acquired without setting on-the-fly instrument compensation parameters. Compensation parameters were determined after acquisition using the FACSDiva software according to the manufacturer's instructions. These instrument settings were applied to each sample. Fluorophore conjugates used in these studies were Allophycocyanin (APC), AlexaFluor 647 (AF647), Fluorescein isothiocyanate (FITC), Phycoerythrin (PE) and Peridinin chlorophyll protein (PerCP), all from BD Biosciences. Table 2 summarizes the expression of selected cell-surface markers, including angiogenic markers.

TABLE 2
Cell surface marker expression in amnion derived adherent cells
as determined by flow cytometry.

| | | | Immuno- |
|---|---|---|---|
| | | | localization |
| | | | Flow |
| AMDAC Marker | Positive | Negative | Cytometry |
| CD6 | | X | X |
| CD9 | X | | X |
| CD10 | X | | X |
| CD31 | | X | X |
| CD34 | | X | X |
| CD44 | X | | X |
| CD45 | | X | X |
| CD49b | X | | X |
| CD49c | X | | X |
| CD49d | X | | X |
| CD54 | X | | X |
| CD68 | X | | X |
| CD90 | X | | X |
| CD98 | X | | X |
| CD105 | X | | X |
| CD117 | | X | X |
| CD133 | | X | X |
| CD143 | | X | X |
| CD144 | | X | X |
| (VE-cadherin) | | | |
| CD146 | | X | X |
| CD166 | X | | X |
| CD184 | | X | X |
| CD200 | X | | X |
| CD202b | X | | X |
| CD271 | | X | X |
| CD304 | X | | X |
| CD309 | X | | X |
| (VEGFR2/KDR) | | | |
| CD318 | X | | X |
| CD349 | X | | X |
| CytoK | X | | X |
| HLA-ABC+ B2 | X | | X |
| Micro+ | | | |
| Invariant Chain+ | | X | X |
| HLA-DR-DP-DQ+ | | | |
| PDL-1 | X | | X |
| VEGFR1/FLT-1 | X | | X |
| | | | X |

Column “Immunolocalization Flow Cytometry” indicates that the presence or absence of particular markers were determined by immunolocalization, specifically flow cytometry.

In another experiment, AMDAC cells were labeled with anti-human CD49f (Clone GoH3, phycoerythrin-conjugated; BD Pharmingen Part No. 555736), and analyzed by flow cytometry. Approximately 96% of the AMDACs labeled with anti-CD49f (that is, were CD49f⁺).

In other experiments, AMDACs were additionally found by immunolocalization to express CD49a, CD106, CD119, CD130, c-met (hepatocyte growth factor receptor; HGFR), CXC chemokine receptor 1 (CXCR1), PDGFRA, and PDGFRB by immunolocalization. AMDACs were also found, by immunolocalization, to lack expression of CD49e, CD62E, fibroblast growth factor receptor 3 (FGFR3), tumor necrosis factor receptor superfamily member 12A (TNFRSF12A), insulin-like growth factor 1 receptor (IGF-1R), CXCR2, CXCR3, CXCR4, CXCR6, chemokine receptor 1 (CCR1), CCR2, CCR3, CCR4, CCR5, CCR6, CCR7, CCR8, CCR9, epidermal growth factor receptor (EGF-R), insulin receptor (CD220), interleukin receptor 4 (IL4-R; CD124), IL6-R (CD126), TNF-R1a and 1b (CD120a, b), and erbB2/Her2.

6.2.3 Immunohistochemistry (IHC)/Immunofluorochemistry (IFC) for Evaluation of Angiogenic Potency of Amnion Derived Adherent Cells

Amnion derived adherent cells from passage 6 were grown to approximately 70% confluence on 4-well chamber slides and fixed with a 4% formalin solution for 30 minutes each. After fixation, the slides were rinsed with PBS two times for 5 minutes. The slides were then incubated with 10% normal serum from the same host as the secondary antibody, 2× casein, and 0.3% Triton X100 in PBS, for 20 minutes at room temperature in a humid chamber. Excess serum was blotted off and the slides were incubated with primary antibody (goat polyclonal IgG (Santa Cruz; Santa Cruz, Calif.) in a humidified chamber. Time and temperature for incubations were determined by selecting the optimal conditions for the antibody being used. In general, incubation times were 1 to 2 hours at 37° C. or overnight at 4° C. The slides were then rinsed with PBS three times for 5 minutes each and incubated for 20-30 minutes at room temperature in a humid chamber with fluorescent-conjugated anti-immunoglobulin secondary antibody directed against the host of the primary antibody (rabbit anti-goat antibody (Santa Cruz)). Thereafter, the slides were rinsed with PBS three times for 5 minutes each, mounted with a coverslip utilizing DAPI VECTASHIELD® (Vector Labs) mounting solution to counterstain nuclei. Cell staining was visualized utilizing a Nikon fluorescence microscope. All pictures were taken at equal exposure time normalized against the background of the corresponding isotype (goat IgG (Santa Cruz)). Table 3 summarizes the results for the expression of angiogenic proteins by amnion derived adherent cells.

TABLE 3
Angiogenic markers present or absent on amnion derived adherent cells.

| | | | Immunolocalization |
|---|---|---|---|
| AMDAC | | | Immunofluorescence |
| Marker | Positive | Negative | Immunohistochemistry |
| CD31 | | X | X |
| CD34 | | X | X |
| (VEGFR2/KDR) | X | | X |
| Connexin-43 | X | | X |
| Galectin-1 | X | | X |
| TEM-7 | X | | X |

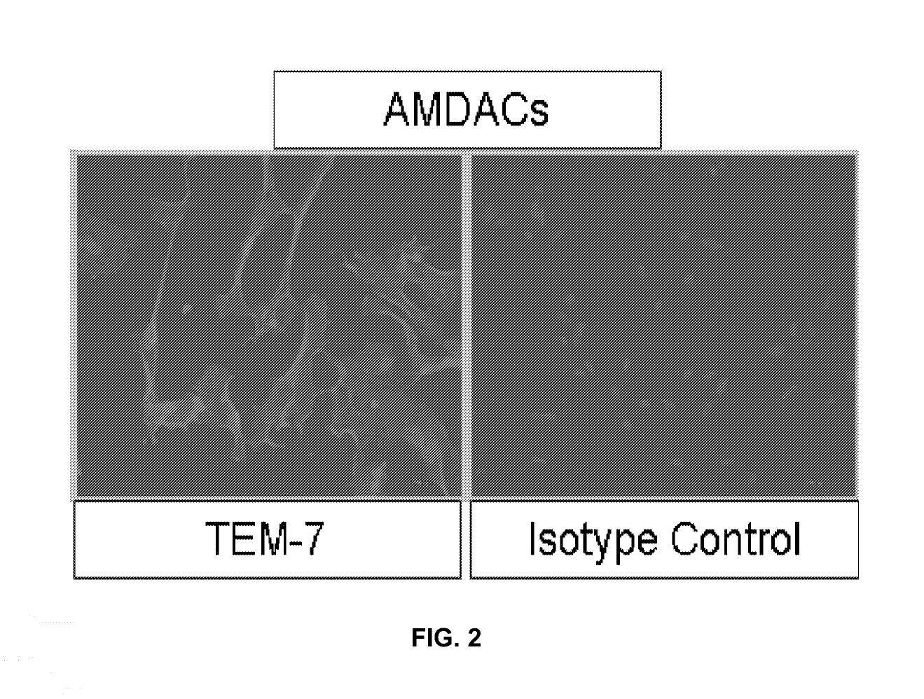

Amnion derived adherent cells expressed the angiogenic marker tumor endothelial marker 7 (TEM-7), one of the proteins shown in Table 3. See FIG. 2.

6.2.4 Membrane Proteomics for Evaluation of Angiogenic Potency of Amnion Derived Adherent Cells

Membrane Protein Purification:

Cells at passage 6 were grown to approximately 70% confluence in growth medium, trypsinized, and washed in PBS. The cells were then incubated for 15 minutes with a solution containing protease inhibitor cocktail (P8340, Sigma Aldrich, St. Louis, Mo.) prior to cell lysis. The cells were then lysed by the addition of a 10 mM HCl solution (thus avoiding the use of detergents) and centrifuged for 10 minutes at 400 g to pellet and remove the nuclei. The post-nuclear supernatant was transferred to an ultracentrifugation tube and centrifuged using a WX80 ultracentrifuge with a T-1270 rotor (Thermo Fisher Scientific, Asheville, N.C.) at 100,000 g for 150 minutes generating a membrane protein pellet.

Generation, Immobilization and Digestion of Proteoliposomes:

The membrane protein pellet was washed several times using Nanoxis buffer (10 mM Tris, 300 mM NaCl, pH 8). The membrane protein pellet was suspended in 1.5 mL of Nanoxis buffer and then tip-sonicated using a VIBRA-CELL™ VC505 ultrasonic processor (Sonics & Materials, Inc., Newtown, Conn.) for 20 minutes on ice. The size of the proteoliposomes was determined by staining with FM1-43 dye (Invitrogen, Carlsbad, Calif.) and visualization with fluorescence microscopy. The protein concentration of the proteoliposome suspension was determined by a BCA assay (Thermo Scientific). The proteoliposomes were then injected onto an LPI™ Flow Cell (Nanoxis AB, Gothenburg, Sweden) using a standard pipette tip and allowed to immobilize for 1 hour. After immobilization, a series of washing steps were carried out and trypsin at 5 μg/mL (Princeton Separations, Adelphi, N.J.) was injected directly onto the LPI™ Flow Cell. The chip was incubated overnight at 37° C. and the tryptic peptides were eluted from the LPI™ chip and then desalted using a Sep-Pak cartridge (Waters Corporation, Milford, Mass.).

LTQ Linear Ion Trap LC/MS/MS Analysis:

Each tryptic digest sample was separated on a 0.2 mm×150 mm 3 μm 200 Å MAGIC C18 column (Michrom Bioresources, Inc., Auburn, Calif.) that was interfaced directly to an axial desolvation vacuum-assisted nanocapillary electrospray ionization (ADVANCE) source (Michrom Bioresources, Inc.) using a 180 minute gradient (Buffer A: Water, 0.1% Formic Acid; Buffer B: Acetonitrile, 0.1% Formic Acid). The ADVANCE source achieves a sensitivity that is comparable to traditional nanoESI while operating at a considerably higher flow rate of 3 μL/min. Eluted peptides were analyzed on an LTQ linear ion trap mass spectrometer (Thermo Fisher Scientific, San Jose, Calif.) that employed ten data-dependent MS/MS scans following each full scan mass spectrum. Seven analytical replicate datasets were collected for each biological sample.

Bioinformatics:

Seven RAW files corresponding to the 7 analytical replicate datasets that were collected for each cell line were searched as a single search against the IPI Human Database using an implementation of the SEQUEST algorithm on a Sorcerer Solo™ workstation (Sage-N Research, San Jose, Calif.). A peptide mass tolerance of 1.2 amu was specified, oxidation of methionine was specified as a differential modification, and carbamidomethylation was specified as a static modification. Scaffold software implementation of the Trans-Proteomic Pipeline (TPP) was used to sort and parse the membrane proteomic data. Proteins were considered for analysis if they were identified with a peptide probability of 95%, protein probability of 95% and 1 unique peptide. Comparisons between membrane proteomic datasets were made using custom Perl scripts developed in-house.

Results:

As shown in Table 4, amnion derived adherent cells expressed various angiogenic and cardiomyogenic markers.

TABLE 4
Cardiomyogenic or angiogenic markers expressed by
amnion derived adherent cells.

| | | | Immunolocalization |
|---|---|---|---|
| | | | Membrane |
| AMDACSMarker | Positive | Negative | Proteomics |
| Activin receptor | X | | X |
| type IIB | | | |
| ADAM 17 | X | | X |
| Alpha-actinin 1 | X | | X |
| Angiotensinogen | X | | X |
| Filamin A | X | | X |
| Macrophage | X | | X |
| acetylated LDL | | | |
| receptor I and II | | | |
| Megalin | X | | X |
| Myosin heavy | X | | X |
| chain non | | | |
| muscle type A | | | |
| Myosin-binding | X | | X |
| protein C cardiac | | | |
| type | | | |
| Wnt-9 | X | | X |

6.2.5 Secretome Profiling for Evaluation of Angiogenic Potency of Amnion Derived adherent cells

Protein Arrays:

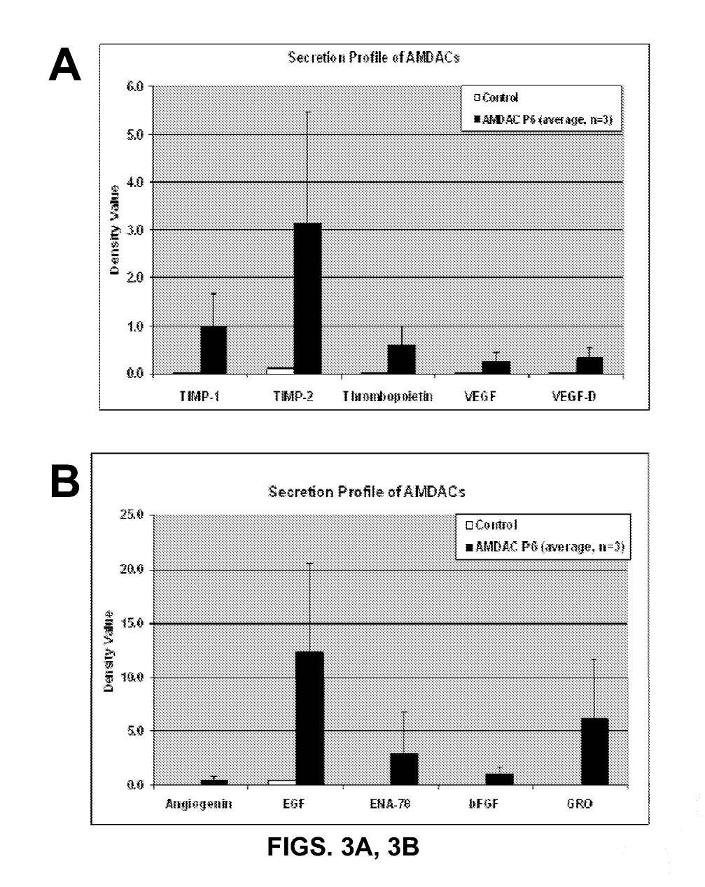

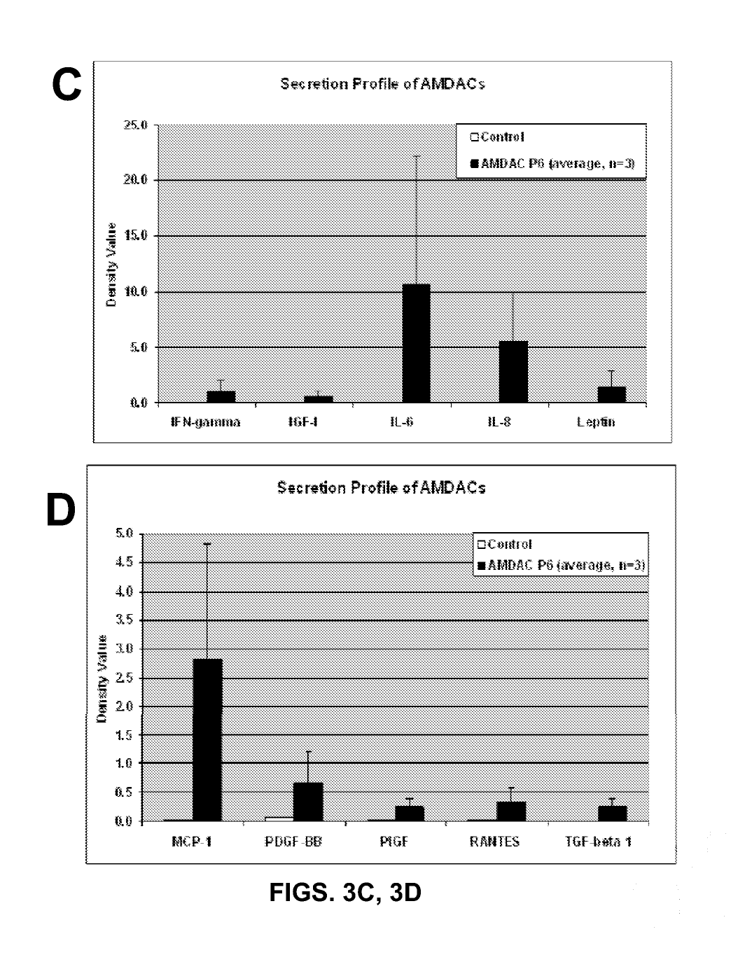

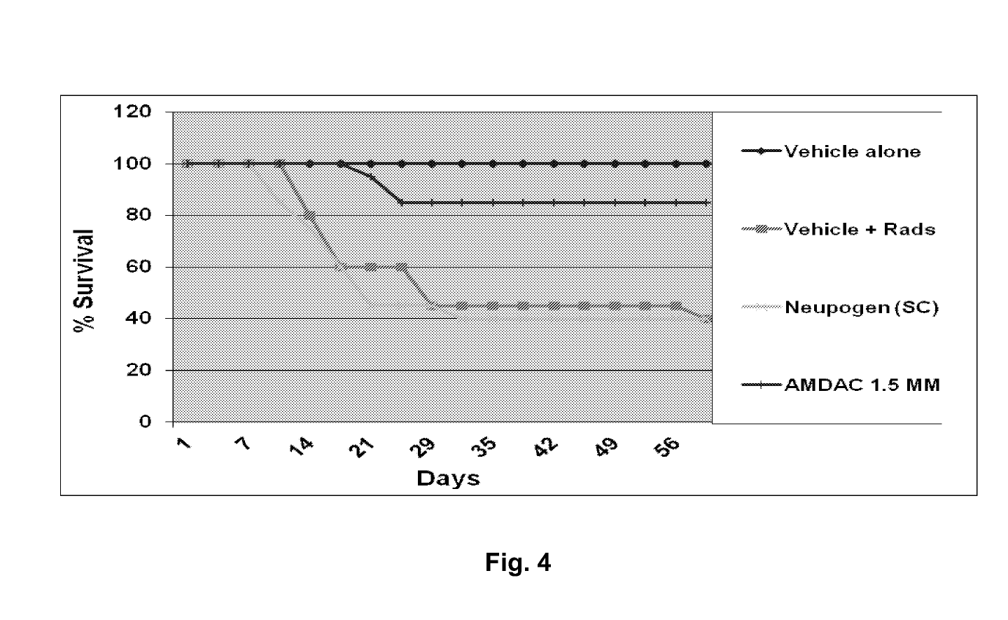

Amnion derived adherent cells at passage 6 were plated at equal cell numbers in growth medium and conditioned media were collected after 4 days. Simultaneous qualitative analysis of multiple angiogenic cytokines/growth factors in cell-conditioned media was performed using RayBiotech Angiogenesis Protein Arrays (Norcross, Ga.). In brief, protein arrays were incubated with 2 mL 1× Blocking Buffer (Ray Biotech) at room temperature for 30 minutes (min) to block membranes. Subsequently, the Blocking Buffer was decanted and the membranes were incubated with 1 mL of sample (growth medium conditioned by the respective cells for 4 days) at room temperature for 1 to 2 hours. The samples were then decanted and the membranes were washed 3×5 min with 2 mL of 1× Wash Buffer I (Ray Biotech) at room temperature with shaking. Then, the membranes were washed 2×5 min with 2 mL of 1× Wash Buffer II (Ray Biotech) at room temperature with shaking Thereafter, 1 mL of diluted biotin-conjugated antibodies (Ray Biotech) was added to each membrane and incubated at room temperature for 1-2 hours and washed with the Wash Buffers as described above. Diluted HRP-conjugated streptavidin (2 mL) was then added to each membrane and the membranes were incubated at room temperature for 2 hours. Finally, the membranes were washed again, incubated with the ECL™ detection kit (Amersham) according to specifications and the results were visualized and analyzed using the Kodak Gel Logic 2200 Imaging System. The secretion of various angiogenic proteins by AMDACs is shown in FIGS. 3A-3D.

ELISAs:

Quantitative analysis of single angiogenic cytokines/growth factors in cell-conditioned media was performed using commercially available kits from R&D Systems (Minneapolis, Minn.). In brief, ELISA assays were performed according to manufacturer's instructions and the amount of the respective angiogenic growth factors in the conditioned media was normalized to 1×10⁶ cells. Amnion derived adherent cells (n=6) exhibited approximately 4500 pg VEGF per million cells and approximately 17,200 pg IL-8 per million cells.

TABLE 5
ELISA results for angiogenic markers

| | | | Secretome |
|---|---|---|---|
| | | | Analysis ELISA, |
| AMDAC Marker | Positive | Negative | Protein Arrays |
| ANG | X | | X |
| EGF | X | | X |
| ENA-78 | X | | X |
| FGF2 | X | | X |
| Follistatin | X | | X |
| G-CSF | X | | X |
| GRO | X | | X |
| HGF | X | | X |
| IL-6 | X | | X |
| IL-8 | X | | X |
| Leptin | X | | X |
| MCP-1 | X | | X |
| MCP-3 | X | | X |
| PDGFB | X | | X |
| PLGF | X | | X |
| Rantes | X | | X |
| TGFB1 | X | | X |
| Thrombopoietin | X | | X |
| TIMP1 | X | | X |
| TIMP2 | X | | X |
| uPAR | X | | X |
| VEGF | X | | X |
| VEGFD | X | | X |

In a separate experiment, AMDACs were confirmed to also secrete angiopoietin-1, angiopoietin-2, PECAM-1 (CD31; platelet endothelial cell adhesion molecule), laminin and fibronectin.

6.2.6 AMDAC MicroRNA Expression Confirms Angiogenic Activity

This Example demonstrates that AMDACs express higher levels of certain micro-RNAs (miRNAs), and lower levels of certain other miRNAs, each of which correlated with angiogenic function, than bone marrow-derived mesenchymal stem cells.

It is known that pro-angiogenic miR-296 regulates angiogenic function through regulating levels of growth factor receptors. For example, miR-296 in endothelial cells contributes significantly to angiogenesis by directly targeting the hepatocyte growth factor-regulated tyrosine kinase substrate (HGS) mRNA, leading to decreased levels of HGS and thereby reducing HGS-mediated degradation of the growth factor receptors VEGFR2 and PDGFRb. See Würdinger et al., Cancer Cell 14:382-393 (2008). In addition, miR-15b and miR-16 have been shown to control the expression of VEGF, a key pro-angiogenic factor involved in angiogenesis, and that hypoxia-induced reduction of miR-15b and miR-16 contributes to an increase in VEGF, a pro-angiogenic cytokine See Kuelbacher et al., Trends in Pharmacological Sciences, 29(1):12-15 (2007).

AMDACs were prepared as described in Example 1, above. AMDACs and BM-MSC cells (used as a comparator) were subjected to microRNA (miRNA) preparation using a MIRVANA™ miRNA Isolation Kit (Ambion, Cat#1560). 0.5×10⁶ to 1.5×10⁶ cells were disrupted in a denaturing lysis buffer. Next, samples were subjected to acid-phenol+chloroform extraction to isolate RNA highly enriched for small RNA species. 100% ethanol was added to bring the samples to 25% ethanol. When this lysate/ethanol mixture was passed through a glass fiber filter, large RNAs were immobilized, and small RNA species were collected in the filtrate. The ethanol concentration of the filtrate was then increased to 55%, and the mixture was passed through a second glass fiber filter where the small RNAs became immobilized. This RNA was washed, and eluted in a low ionic strength solution. The concentration and purity of the recovered small RNA was determined by measuring its absorbance at 260 and 280 nm.

AMDACs were found to express the following angiogenic miRNA: miR-17-3p, miR-18a, miR-18b, miR-19b, miR-92, miR-20a, miR-20b, (members of the of the angiogenic miRNA cluster 17-92), miR-296, miR-221, miR-222, miR-15b, miR-16. AMDACs were also found to express higher levels of the following angiogenic miRNA when compared to bone marrow-derived mesenchymal stem cells (BM-MSCs): miR-17-3p, miR-18a, miR-18b, miR-19b, miR-92 (members of the of the angiogenic miRNA cluster 17-92), miR-296. These results correlate well with the observation that AMDACs express high levels of VEGFR2/KDR (see above). Conversely, AMDACs were found to express lower levels of the following angiogenic miRNA when compared to BM-MSCs: miR-20a, miR-20b, (members of the of the angiogenic miRNA cluster 17-92), miR-221, miR-222, miR-15b, miR-16. The reduced expression of miR-15b and miR-16 correlated with the higher levels of expression of VEGF seen in AMDACs.

### 6.3 Example 3

### Treatment of Radiation Damage Using AMDACS

The objective of this study was to assess the LD60/60 in mice following a single acute whole body exposure of gamma irradiation using a cesium source (Cs-γ irradiation) with or without treatment with either AMDACs or a reference positive control (Neupogen®) administered 24 hr post-irradiation. Comparative analysis of improvement in survival due to AMDACs was evaluated as compared with the non-irradiated vehicle control group.

In this study, each of four Groups was assigned 29 mice. The 29 mice were further divided by designating 9 of the mice as a Satellite group for interim necropsy on Days 4, 15 and 29 (3 mice/day). Mice from this Satellite group were established for collection of blood for hematology analysis and the collection of tissues for possible future PCR evaluation. The remaining 20 mice were designated as the Main group and assigned for necropsy on Day 60. Group 1 was not treated with Cs-γ irradiation while all remaining groups (i.e., Groups 2, 3, and 4) received a single irradiation dose of 940 cGy on Day 0. Groups treated with vehicle, cells or positive control post-irradiation were given a single intravenous, single subcutaneous, or multiple iv administration as follows: Groups 1 and 2 received vehicle alone; multiple dose administrations were given for Group 3, which received 200 μg/kg of Neupogen® (sc) on Days 1-5; and group 4 received AMDACs (iv) at 1.0×10⁶ total viable nucleated cells (TNC) on Day 1. Day 1 represents 24-hours after administration of radiation. See Table 6.

TABLE 6
Administration Scheme Per Group

| GROUP # | RADIATION | NEUPOGEN ® (SC) | AMDACS (IV) |
|---|---|---|---|
| 1 (Vehicle Control) | None | None | None |
| 2 (Radiation Control) | 940 cGy on Day 0 | None | None |
| 3 (Positive Control) | 940 cGy on Day 0 | 200 μg/kg; Days 1-5 | None |
| 4 | 940 cGy on Day 0 | None | 1.0 × 10⁶ TNC on Day 1 |

No morbidity or mortality was observed for mice assigned to the Main or Satellite animals from Group 1. Three mice died in Group 3; and moribund mice sacrificed early totaled 1 and 2 from Groups 2 and 4, respectively. Similarly, deaths were seen in Satellite mice with 3, 3 and 1 mice found dead in Groups 2, 3 and 4, respectively. Clinical signs that were attributed to dose treatment and/or radiation included findings of hunched posture, hypothermia, emaciation, hypoactivity, dyspnea, and ruffled fur that were seen in either or both Main and Satellite groups. These findings were considered to be toxicologically limiting and a result from administration of radiation alone or in combination with dose treatment. These observations notwithstanding, statistically significantly survival rate comparisons across the four groups studied were successfully generated, as discussed below.

Statistically significant decreases in mean weight were seen in all groups that received radiation treatment. Decreases in weight in the Main and Satellite groups were observed following radiation and lasting at least up to Day 11 and as long as Day 60, with sporadic decreases seen on subsequent day in some groups.

The increases in clinical signs of hunched posture, hypothermia, emaciation, hypoactivity, dyspnea, ruffled fur, and decreased body weight, noted above, were all attributed to dose and/or radiation treatment that were considered to be toxicologically limiting. Analysis of blood cell immune levels of satellite animals confirmed neutropenia in animals exposed to radiation.

The survival curves over the course of the study for animals within each group are presented in FIG. 4. Statistical analysis of survival was evaluated in two ways: first by comparing the treatment groups (Groups 3 and 4) with untreated (naïve) Group 1, and second by comparing the treatment groups with Group 2, which was exposed to 940 cGy Cs-γ irradiation alone, and which represented a radiation control group.

Vehicle control Group 1 compared with Group 2 animals, which received radiation alone, showed a statistically significantly difference in survival rate (p<0.001). A single radiation dose of 940 cGy was lethal to 50% of the animals in Group 2 at 30 days post irradiation (see FIG. 4).

Vehicle control Group 1 animals compared with Group 3 animals, which were treated with Neupogen® 24 h post irradiation, also showed a statistically significantly difference in survival rate (p<0.001). Neupogen® (filgrastim) is a granulocyte colony-stimulating factor (G-CSF) analog used to stimulate the proliferation and differentiation of granulocytes, and used in the treatment of neutropenia (see, e.g., Beveridge et al., 1988, Cancer Invest. 16 (6):366-73). Despite treatment of the irradiated mice in Group 3 with Neupogen® daily for five days after radiation, a single radiation dose of 940 cGy was lethal to 50% of the animals in Group 2 at −20 days post irradiation (see FIG. 4). Rather, mice treated with Neupogen® demonstrated a survival rate comparable to mice that were irradiated but received no subsequent treatment (i.e., the mice of Group 2; see FIG. 4).

In contrast, Group 4 animals, which were exposed to radiation then treated with AMDACs 24-hours post-radiation, did not show a statistically significantly difference in survival rate compared to the vehicle control group animals (Group 1). As shown in FIG. 4, administration of a single dose of AMDACs to mice exposed to radiation at LD₅₀ was sufficient to promote the survival of >80% of the animals. Further, mice treated with AMDACs subsequent to exposure to radiation (i.e., Group 4 animals) also showed a statistically significantly difference in survival rate (p=0.003) from Group 2 animals, which were irradiated but received no subsequent treatment.

In conclusion, administration of AMDACs to mice following exposure to lethal doses of radiation promotes a survival rate of greater than 80%, whereas mice exposed to radiation alone, or mice exposed to radiation and subsequently treated with Neupogen®, demonstrated a survival rate of less than 50%.

### 6.4 Example 4

### AMDACs Induce Hematopoietic Reconstitution

The objective of this study was to confirm and expand upon the study described in Example 3, above. Treatment efficacy was evaluated following a single intravenous dose administration of two concentrations of AMDACs in mice after receiving a single dose of γ-irradiation. The impact of AMDAC treatment on radiation-induced neutropenia and several other endpoints, including immune responses were also evaluated.

6.4.1 Methods

6.4.1.1 Experimental Groups and Administration Protocol

This study consisted of 4 study groups (1-4) that were further divided into Main and Satellite subgroups. Group 1 animals were not exposed to radiation, but received a single dose of vehicle buffer solution. Four animals were assigned to the Main subgroups and 8 animals to the Satellite subgroups for Group 1. For Groups 2-4, twenty animals each were assigned to the Main subgroups and eight animals each were assigned to the Satellite subgroups. The animals of Groups 2-4 received a single dose of 940 cGy γ-irradiation on Day 0. Irradiated Group 2 animals received a single dose of vehicle buffer solution; irradiated animals from Groups 3 and 4 were administered one intravenous (iv) dose each of total nucleated cells (TNC) of AMDACs approximately 24 hr following radiation treatment (Day 1). See Table 7. Surviving animals from the Main groups were necropsied on Day 60; and 4 animals each from the Satellite groups of Groups 1-4 were necropsied on Days 14 or 30.

TABLE 7
Administration Scheme Per Group

| GROUP # | RADIATION | AMDACS (IV) |
|---|---|---|
| 1 (Vehicle Control) | None | None |
| 2 (Vehicle/Radiation | 940 cGy on Day 0 | None |
| Control) | | |
| 3 | 940 cGy on Day 0 | 1.25 × 10⁶ TNC on Day 1 |
| 4 | 940 cGy on Day 0 | 2.5 × 10⁶ TNC on Day 1 |

6.4.1.2 Blood Collection and Analysis

Blood was collected from the retro-orbital sinus of mice under isoflurane anesthesia or alternately from the tail vein. Hematology samples were collected using K3-EDTA as the anticoagulant. Anticoagulant was not used for serum chemistry samples collected. A minimum of 500 μl of whole blood was collected for terminal bleeds; blood was collected from the Main animal subgroups (from Groups 1-4) once on Day 60, and from the Satellite animal subgroups (from Groups 1-4) on Days 14 and 30 (4 animals/group/day). Whole blood that was collected was used in the following hematology analyses: hematocrit (HCT), hemoglobin (HGB), red blood cell count (RBC), red blood cell distribution width (RDW), white blood cell count (WBC), WBC differential and absolute counts (absolute neutrophil (ANS), absolute banded neutrophil (ANB), percentage banded neutrophil (PNB), absolute lymphocyte (ALY), percent lymphocyte (PLY), absolute monocyte (AMO), percent monocyte (PMO), absolute eosinophil (AEO), percent eosinophil (PEO), absolute basophil (ABA), and percent basophil (PBA)), mean corpuscular hemoglobin (MCH), mean corpuscular volume (MCV), mean corpuscular hemoglobin concentration (MCC), platelet count (PLC), mean platelet volume (MPV), and reticulocyte count (absolute, REA, and percent, RET).

6.4.1.3 FACS

Animals from Satellite groups were used for the collection of bone marrow for fluorescence-activated cell sorting (FACS).

Both femurs were collected for FACS analysis. The femurs were collected and bone marrow (BM) was isolated by flushing the contents of the femurs into a pre-labeled cryovial (or equivalent) containing ˜300 μl PBS using a 1 cc syringe with ˜25G needle. Collected marrow samples were placed on wet ice until transferred within approximately 1-2 hr to establish viability for FACS immunophenotyping analysis. Bone marrow cells were analyzed by flow cytometry for mouse cell determinants of mouse hematopoietic lineage (CD3, B220, CD11b, Ly-6c, Ter119), c-kit (CD117) and Sca-1, using monoclonal antibodies directed to these markers. This analysis was performed on animals from the Satellite subgroups on Days 14 and 30 of the study. Abbreviations for labeled antibodies are as follows: allophycocyanin (APC), phycoerythrin (PE) and cyanine (Cy). Staining buffer was prepared by adding bovine serum albumin (BSA; 1%) to PBS. Cells were stained in a 96-well plate using fluorescently-labeled monoclonal antibodies as described in Table 8, below.

TABLE 8

| Fluorochrome | Marker(s) |
|---|---|
| V450 | CD117 |
| PerCP-Cy 5.5 | Mouse hematopoietic |
| | lineage determinants |
| PE | Sca1 |

For staining, the cells were plated in a round-bottom, 96-well microtiter plate (1×10⁶ cells/well). The cells were washed with cold staining buffer (100 μl/well; 1% BSA in PBS) by centrifuging the plate at 300×g for 5 min at 4° C. and decanting the supernatants. Mouse IgG (5 μg) was added into each well, and after 5-7 min of incubation at 2-8° C., the antibody mixtures (40 μl antibody mixtures per well) were added into each well. After 30 to 35 min of refrigeration, the stained cells were washed two times with cold staining buffer (150-200 μl/well). After staining, samples were analyzed using an LSR-II flow cytometer (Becton Dickinson).

6.4.2 Results

6.4.2.1 Survival Study

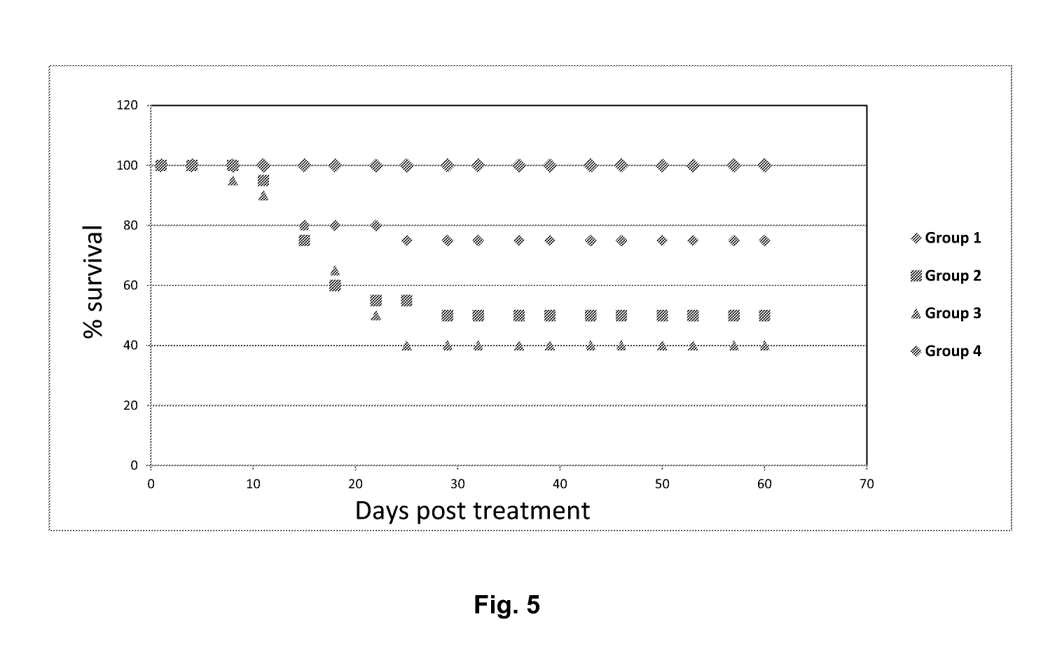

The survival curves over the 60-day course of the study for animals within each group are presented in FIG. 5. As shown in FIG. 5, the percentage of mice that survived to Day 60 were 100%, 50%, 40%, and 75% for Groups 1-4 respectively. The rate of animal deaths observed for animals in Group 2 (940 cGy Cs-γ) was 1, 4, 3, 1 and 1 deaths by Days 11, 15, 18, 22 and 29; for Group 3 (940 cGy Cs-γ and 1.25×10⁶ TNC AMDACs) was 1, 1, 2, 3, 3 and 2 deaths by Days 8, 11, 15, 18, 22 and 25; and for Group 4 (940 cGy Cs-γ and 2.5×10⁶ TNC AMDACs) was 4 and 1 deaths by Days 15 and 25, respectively.

Statistical analysis of survival was evaluated in two ways, first by comparing Groups 2-4 with the non-irradiated vehicle control group (Group 1), and then by comparing treatment Groups 3 and 4 with Group 2, the animals of which were exposed to radiation and treated with vehicle only. Statistical analysis was also evaluated between Groups 3 and 4, the animals of which received different doses of AMDACs (1.25×10⁶ TNC and 2.5×10⁶ TNC, respectively).

Due to the small number of animals in vehicle control Group 1, no statistical difference was determined between survival data from any of the groups when compared to Group 1. However, FIG. 5 clearly shows that a single radiation dose of 940 cGy was lethal to 50% of the mice in Group 2 by Day 29 post-irradiation, consistent with the results described in Example 3, above. Further, FIG. 5 demonstrates that while survival of mice treated 1.25×10⁶ AMDACs (Group 3) was comparable to that of the untreated mice of Group 2, mice receiving a higher dose of AMDACs (i.e., 2.5×10⁶) demonstrated better survival that the mice of Group 2, which were irradiated but received treatment thereafter.

6.4.2.2 Pathological Analyses

Evaluation of hematology parameters for the Satellite animals on Days 14 and 30 were compared. Each of Groups 2-4 showed statistically significant decreases in hematocrit (HCT), hemoglobin (HGB), red blood cells (RBC), white blood cells (WBC), total neutrophils (ANS), total lymphocytes (ALY), platelet count (PLC), absolute reticulocyte count (REA) and percent reticulocyte (RET) when compared to Group 1. Except for ALY and PLC, all the parameters that were reduced on Day 14 were comparable to the vehicle control levels by Day 30.

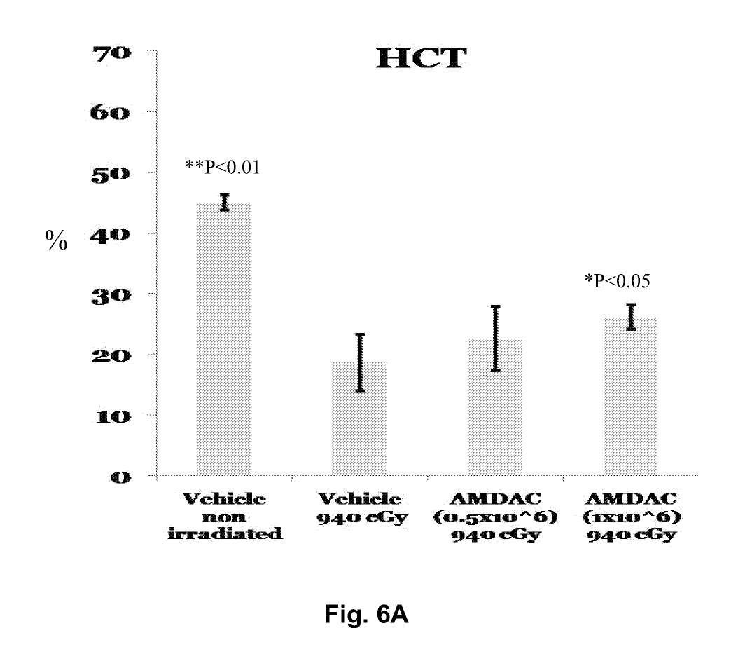

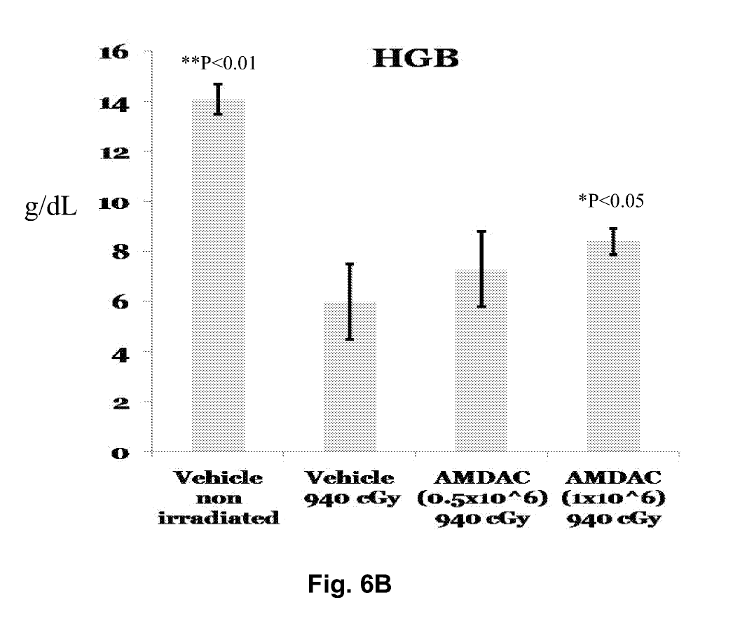

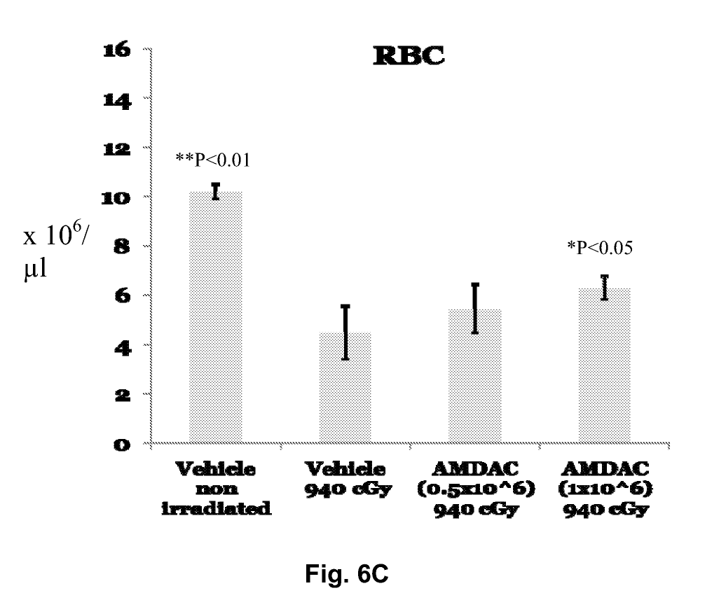

The results of the hematological analyses obtained for Group 2 were compared with the results obtained for Groups 3 and 4 (the groups treated with different doses of AMDACs following radiation exposure). Significantly different results were observed when certain parameters were compared. Specifically, Group 4 animals demonstrated statistically significant increases in HCT, HGB, and RBC values on Day 14 as compared those levels observed in Group 2. See FIG. 6A-C. By Day 30, the values were comparable to Group 2.

6.4.2.3 FACS Results

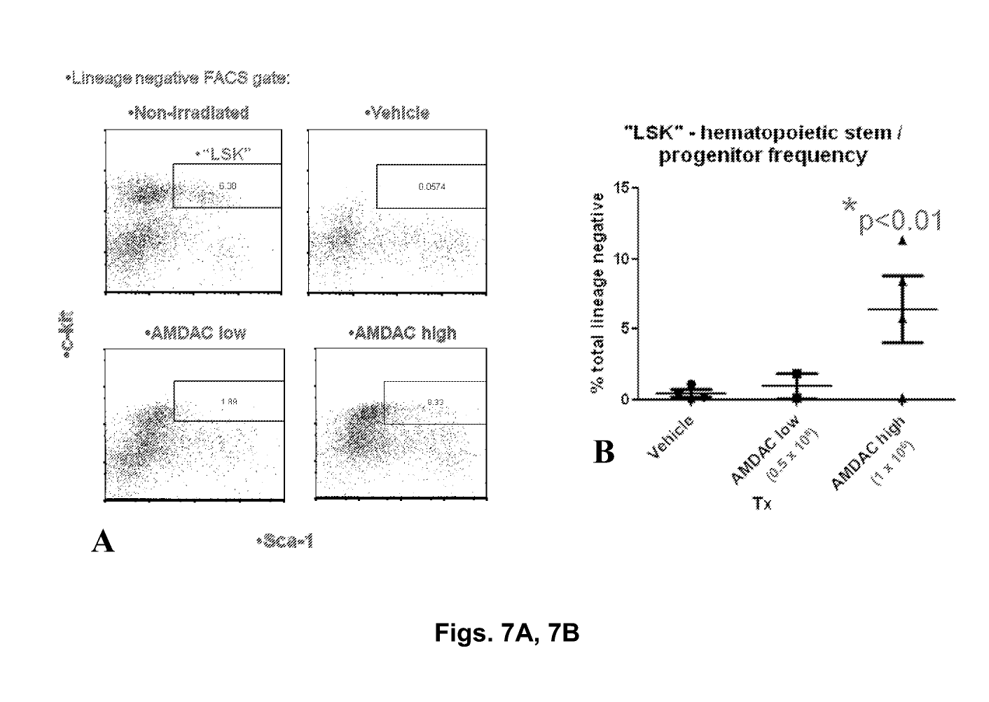

FACS analysis of the murine bone marrow lineage negative, C-kit+/Sca-1+ cell population (LSK cells) was performed on the Satellite animals of Groups 1-4 at Days 14 and 30. See FIG. 7A. This population is normally highly enriched in hematopoietic stem and primitive multilineage progenitor cells. As expected, the frequency of hematopoietic stem and primitive progenitor cells (HSC and HPPC) was profoundly decreased in the irradiated control group (Group 2) as compared to the non-irradiated control group (Group 1), at both time points tested. Specifically, as shown in FIG. 7A, 6.38% of the tested cells of Group 1 mice, which were not exposed to radiation, were C-kit+/Sca-1+, whereas only 0.0574% of the tested cells of Group 2 (radiation exposed) mice were C-kit+/Sca-1+. As also shown in FIG. 7A, AMDAC-treated mice (Groups 3 and 4) had much higher levels of C-kit+/Sca-1+ cells compared to the levels of such cells in the mice of Group 2: 1.89% of the tested cells of mice treated with 1.25×10⁶ AMDACs (Group 3) were C-kit+/Sca-1+ and 8.33% of the tested cells of mice treated with 2.5×10⁶ AMDACs (Group 4) were C-kit+/Sca-1+. In fact, mice treated with the higher dose of AMDACs following radiation exposure had numbers of C-kit+/Sca-1+ cells comparable to those of mice that were not exposed to radiation (Group 1). This data is corroborated in FIG. 7B, which shows that, at the first time point (14 days following irradiation), significantly higher frequencies of HSC and HPPC were detected in Group 4 (AMDAC-treated mice) as compared to the vehicle treated irradiated animals of Group 2.

6.4.3 Conclusions

Administration of AMDACs following exposure to radiation promotes prolonged survival. Further, such administration causes significant, beneficial changes in hematology parameters. These findings indicate that AMDACs treatment can reduce the severity of myelosuppression, which can contribute to the survival observed in AMDAC-treated mice following lethal exposure to radiation. Moreover, the FACS results demonstrate a direct correlation of endogenous hematopoietic stem and progenitor cell frequency with the enhanced survival rate observed with a high dose of AMDACs compared to the control irradiated animals. The observed effects of AMDACs on endogenous stem cells are consistent with the induction of endogenous hematopoietic stem cell repair and not anti-apoptotic effects, as the therapy was administered 24 h following the exposure to radiation, which is outside of the window of efficacy for cytoprotective agents. Thus, the data indicate that AMDACs treat radiation injury via a mechanism that involves hematopoietic reconstitution.

### EQUIVALENTS

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

1. A method of treating an individual who has been exposed to radiation, comprising administering to the individual a therapeutically-effective amount of isolated amnion derived adherent cells (AMDACs), wherein said cells are adherent to tissue culture plastic, and wherein said cells are OCT-4⁻ (octamer binding protein 4) as determinable by RT-PCR.

2. The method of claim 1, wherein said radiation is ionizing radiation, beta radiation, gamma radiation, or X-rays.

3-5. (canceled)

6. The method of claim 1, wherein said radiation is a single dose or a chronic exposure.

7-23. (canceled)

24. The method of claim 6, wherein said chronic exposure is over 1-6 days, 7-13 days, 14-27 days, or 28-56 days.

25-27. (canceled)

28. The method of claim 1, wherein said individual has developed, or is likely to develop, acute radiation syndrome or a symptom of acute radiation syndrome as a result of said exposure to radiation.

29. The method of claim 1, wherein said individual has not yet developed one or more symptoms of acute radiation syndrome at the time of said administering.

30. The method of claim 28, wherein said one or more symptoms comprise one or more of nausea, vomiting, diarrhea, fever, headache, purpuria, weakness, fatigue, infections, alopecia, blistering or necrosis of exposed tissue, hemorrhage, neurological impairment, cognitive impairment, ataxia, tremors, seizures, or leukopenia.

31-33. (canceled)

34. The method of claim 1, wherein said individual is exposed to radiation from a source not contacting the individual's body.

35. The method of claim 1, wherein said individual is exposed to radiation as a result of a radioactive source contacting the individual's body.

36. The method of claim 1, wherein said individual is exposed to radiation as a result of the individual's inhalation or ingestion of a radioactive source.

37. The method of claim 1, wherein said administering takes place within 96 hours of said exposure, within 72 hours of said exposure, within 48 hours of said exposure, or within 24 hours of said exposure.

38-40. (canceled)

41. The method of claim 1, wherein said AMDACs are HLA-G⁻, as determinable by RT-PCR.

42. The method of claim 1, wherein said AMDACs are CD49f⁺, as determinable by flow cytometry.

43. (canceled)

44. The method of claim 1, wherein said AMDACs are CD90⁺, CD105⁺, or CD117⁻ as determinable by flow cytometry.

45. (canceled)

46. (canceled)

47. The method of claim 1, wherein said AMDACs are VEGFR1/Flt-1⁺ (vascular endothelial growth factor receptor 1) and VEGFR2/KDR⁺ (vascular endothelial growth factor receptor 2), as determinable by immunolocalization.

48. The method of claim 1, wherein said AMDACs are one or more of CD9⁺, CD10⁺, CD44⁺, CD54⁺, CD98⁺, Tie-2⁺ (angiopoietin receptor), TEM-7⁺ (tumor endothelial marker 7), CD31⁻, CD34⁻, CD45⁻, CD133⁻, CD143⁻ (angiotensin-I-converting enzyme, ACE), CD 146⁻ (melanoma cell adhesion molecule), or CXCR4⁻ (chemokine (C-X-C motif) receptor 4) as determinable by immunolocalization.

49. The method of claim 1, wherein said AMDACs are CD9⁺, CD10⁺, CD44⁺, CD54⁺, CD98⁺, Tie-2⁺ (angiopoietin receptor), TEM-7⁺ (tumor endothelial marker 7), CD31⁻, CD34⁻, CD45⁻, CD133⁻, CD143⁻, CD146⁻, and CXCR4⁻ as determinable by immunolocalization.

50. The method of claim 1, wherein said AMDACs are VE-cadherin⁻ as determinable by immunolocalization.

51. The method of claim 1, wherein said AMDACs are additionally positive for CD105⁺ and CD200⁺ as determinable by immunolocalization.

52. The method of claim 1, wherein said AMDACs do not express CD34 as determinable by immunolocalization after exposure to 50 ng/mL VEGF for 7 days.

53-64. (canceled)
